(19) United States
(12) Patent Application Publication
(10) Pub. No.: US 2009/0082372 A1
(43) Pub. Date: Mar. 26, 2009

(54) Arylmethylene Substituted N-Acyl-Beta-Amino Alcohols

(76) Inventors: Lars WORTMANN, Berlin (DE); Marcus Koppitz, Berlin (DE); Bernd Menzenbach, Jena (DE); Dirk Kosemund, Erfurt (DE); Norbert Schmees, Berlin (DE); Hans-Peter Muhn, Berlin (DE); Thomas Frenzel, Hofhem (DE); Florian Peter Liesener, Trastberg (DE); Anna Schrey, Berlin (DE); Ronald Kuehne, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/179,037

(22) Filed: Jul. 24, 2008

Related U.S. Application Data
(60) Provisional application No. 60/952,037, filed on Jul. 26, 2007.

(30) Foreign Application Priority Data
Jul. 26, 2007 (EP) 07075645.7

(51) Int. Cl. A61K 31/437; C07D 471/04; C07D 409/12; A61K 31/416; A61K 31/4709; A61P 19/10; A61P 15/00; A61K 31/47; C07D 231/56; A61K 31/517

(52) U.S. Cl. 514/266.21; 546/113; 546/165; 546/169; 548/362.5; 544/284; 514/300; 514/406; 514/311; 514/314

(57) ABSTRACT

The present invention relates to arylmethylene substituted N-Acyl-β-amino alcohols of the formula I

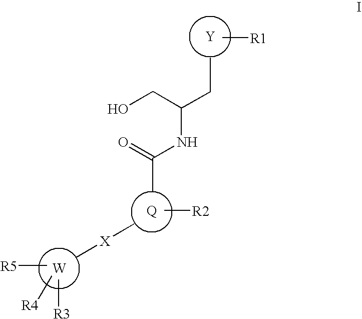

in which
Y is selected from the aryl or heteroaryl groups:

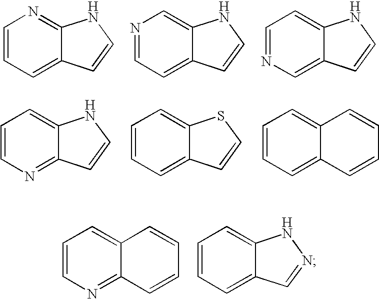

and
R1, R2, R3, R4, R5, Q, X and W have the meaning as defined in the description.

The compounds according to the invention are effective FSH antagonists and can be used for example for fertility control in men or in women, or for the prevention and/or treatment of osteoporosis.

This application claims the benefit of the filing date of U.S. Provisional Application Ser. No. 60/952,037 filed Jul. 26, 2007.

The present invention relates to arylmethylene substituted N-Acyl-β-amino alcohols with FSH-receptor antagonist activity. The present invention also relates to a process for their preparation, pharmaceutical compositions comprising the compounds according to the invention, and the use thereof for fertility control in men or women, for the treatment and/or prevention of osteoporosis.

Follicle-stimulating hormone (FSH) and luteinizing hormone (LH) are together responsible for the control of male and female fertility and of the production of sex steroids.

In the female mammal, FSH controls the early ripening of ovarian primary follicles and the biosynthesis of sex steroids. In the advanced stage of differentiation (preantral follicles), the influence of LH becomes increasingly important for further development of the follicles until ovulation occurs.

In male mammals, FSH is primarily responsible for the differentiation and stimulation of Sertoli cells. Their function consists of assisting spermatogenesis on many levels. LH is primarily responsible for stimulating the Leydig cells and thus androgen production. FSH, LH and TSH (thyrotropic hormone) together form the group of glycoprotein hormones which are formed in the pituitary and are secreted from there. Whereas the alpha subunit is common to the three hormones, their specificity of action is determined by the beta chain which is unique in each case. The molecular weight of FSH including the sugar portion is about 30 kD.

FSH and the other glycoprotein hormones act specifically via their selectively expressed G protein-coupled receptor (GPCR). FSH stimulates, through binding to its receptor, the association thereof with a stimulating G protein (Gₛ) which is thereby stimulated to hydrolyse guanosine triphosphate (GTP) and to activate the membrane-associated adenylate cyclase. Cyclic adenosine monophosphate (cAMP) is accordingly an important and readily quantifiable secondary messenger substance of FSH (G. Vassart, L. Pardo, S. Costagliola, Trends Biochem. Sci. 2004, 29, 119-126).

The importance of FSH for male fertility is the subject of intensive research. It has been possible to show that FSH influences several processes of spermatogenesis such as the proliferation of spermatogonia, the antiapoptotic effect on spermatogonia and spermatocytes and the stimulation of sperm maturation including motility thereof.

The following arguments are also in favour of the FSH receptor as target for male fertility control:
1. The FSH receptor is exclusively expressed on Sertoli cells (high specificity).
2. Contraceptive vaccination against FSH beta chain or the FSH receptor induces infertility in male primates (N. R. Mougdal, M. Jeyakumar, H. N. Krishnamurthy, S. Sridhar, H. Krishnamurthy, F. Martin, Human Reproduction Update 1997, 3, 335-346).
3. Naturally occurring mutations in the FSH receptor or the FSH beta chain may lead to sub- or infertility in men (I. Huhtaniemi, Journal of Reproduction and Fertility 2000, 119, 173-186; L. C. Layman, P. G. McDonough, Molecular and Cellular Endocrinology 2000, 161, 9-17).
4. Neutralizing FSH antiserum has no effect on testis weight and testosterone production (V. Sriraman, A. J. Rao, Molecular and Cellular Endocrionology 2004, 224, 73-82). Adverse effects of FSH blockade on androgen production therefore appear unlikely.

In line with these arguments, FSH antagonists are expected to be suitable for spermatogenesis inhibition (prevention) in men. Moreover, a suitable FSH antagonist may just as well lead to infertility in women, because it suppresses follicle ripening and thus also ovulation. On the other hand, the skilled person expects advantages from non-peptidergic FSH agonists when used to promote fertility in women (stimulation of follicle ripening). There are no reports of experience on the use of FSH or FSH agonists in male infertility, but specific indications are also conceivable in this connection.

New findings demonstrate that there is also a direct effect of FSH on cells of bone metabolism. There are two fundamentally different cell types in bones: osteoclasts and osteoblasts. While osteoclasts play a central role in bone resorption (breakdown of bone), osteoblasts simulate bone density (anabolic effect).

FSH receptors have been detected in osteoclasts but not in osteoblasts. In vitro, FSH stimulates bone resorption by mouse osteoclasts (Li Sun et al. Cell 2006; 125: 247-60). A clinical correlation between the serum FSH level and low bone density has been observed in postmenopausal women (Devleta et al, J. Bone Miner. Metab. 2004, 22: 360-4).

These findings among others suggest that FSH stimulates loss of bone mass, and consequently FSH antagonists will display an antiresorptive effect on bone and are therefore suitable for the therapy and/or prevention of peri- and postmenopausal loss of bone mass and osteoporosis.

FSH receptor modulators are compounds that have a mixed profile of both FSH receptor antagonistic and FSH receptor agonistic properties. FSH receptor modulators of various compound classes of low molecular weight, have been reported on recently. FSH receptor modulators are disclosed in WO 2004/056779, WO 2004/056780; J. Med. Chem. 2005, 48, 1697 [tetrahydroquinolines]; WO 02/70493, Bioorg. Med. Chem. Lett. 2004, 14, 1713 and 1717 [diketopiperazines]; WO 01/47875 [sulphonamides] and EP07090087.3 [hydroxyethyltryptamines].

FSH receptor agonists are disclosed in WO 02/09706; J. Comb. Chem. 2004, 6, 196 [Thiazolidinones]; WO 2003/020726 and WO 03/20727, Chem. Biochem. 2002, 10, 1023 {thieno[2,3-d]pyrimidines)}; WO 01/87287 [pyrazoles]; WO 00/08015 [carbazoles]; WO 06/117023, WO 06/117368, WO 06/117370, WO 06/117371, [hexahydroquinolines].

FSH receptor antagonists are disclosed in WO 03/004028 [tetrahydroquinolines], WO 02/09705 [thiazolidinones], WO 00/58277, Bioorg. Med. Chem. 2002, 10, 639 [sulphonic acids]; WO 00/58276, Endocr. 2002, 143, 3822; Synth. Comm. 2002, 32, 2695 [azo compounds]; US 2006/0199806, US 2006/0258644, US 2006/0258645, US 2006/0287522 [pyrrolobenzodiazepines], WO 2007/017289 [acyltryptophanols], EP06090223.6 [1,2-diarylacetylene derivatives of acyltryptophanols], EP06077263.9 [bicyclic acyltryptophanols], EP07090034.5 [sulfonyltryptophanols] and EP07090059.2 [tetrahydrocarbazoles].

WO 2007/017289 is considered to be the closest prior art.

In view of the prior art, the objective technical problem to be solved according to the present invention may therefore be seen in providing alternative compounds having a FSH receptor antagonistic activity.

The technical problem has been solved according to the present invention by the provision of novel compounds of the formula I

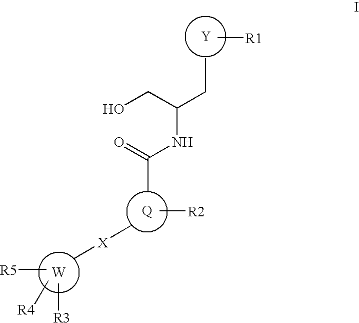

in which
R1 is hydrogen, fluorine, C₁-C₆-alkyl in which the alkyl chain may optionally be substituted one or more times by fluorine;
R2 is hydrogen, halogen, nitro, amino, cyano, C₁-C₆-alkyl, C₂-C₆-alkenyl or C₂-C₆-alkynyl, C₃-C₇-cycloalkyl, hydroxy-C₁-C₆-alkylene, hydroxy-C₃-C₆-alkenylene, hydroxy-C₃-C₆-alkynylene, C₁-C₆-alkyloxy, C₁-C₆-alkyloxy-C₁-C₆-alkylene, C₃-C₇-cycloalkyloxy, C₃-C₇-cycloalkyl-C₁-C₆-alkylenoxy, C₃-C₇-cycloalkyloxy-C₁-C₆-alkylene, C₁-C₆-alkyloxy-C₃-C₆-alkenylene, C₁-C₆-alkyloxy-C₃-C₆-alkynylene, C₁-C₆-alkylamino-C₁-C₆-alkylene, di(C₁-C₆-alkyl)amino-C₁-C₆-alkylene;
   where the hydrocarbon chains therein may optionally be substituted one or more times by fluorine, cyano, hydroxy, amino or by the groups:

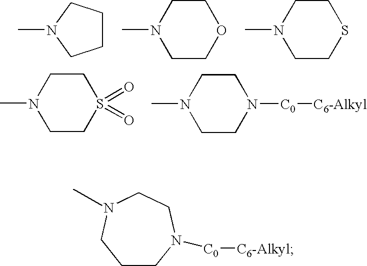

R3, R4, R5 are independently of one another hydrogen, hydroxy, halogen, nitro, amino, cyano, phenyl, C₁-C₆-alkyl, C₂-C₆-alkenyl or C₂-C₆-alkynyl, C₃-C₇-cycloalkyl, C₃-C₇-cycloalkyl-C₁-C₆-alkylene, C₃-C₇-heterocycloalkyl, where the hydrocarbon chains therein may optionally be substituted one or more times by fluorine, cyano or by the radicals:

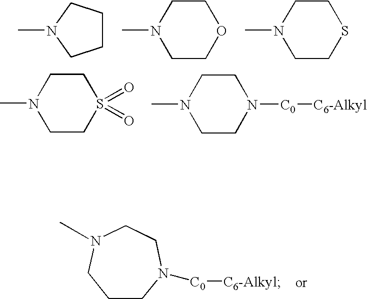

independently of one another hydroxy-C₁-C₆-alkylene, hydroxy-C₃-C₆-alkenylene, hydroxy-C₃-C₆-alkynylene, C₁-C₆-alkyloxy, C₃-C₇-cycloalkyloxy, C₃-C₇-cycloalkyl-C₁-C₆-alkylenoxy, C₁-C₆-alkyloxy-C₁-C₆-alkylene, C₃-C₇-cycloalkyloxy-C₁-C₆-alkylene, C₁-C₆-alkyloxy-C₃-C₆-alkenylene, C₁-C₆-alkyloxy-C₃-C₆-alkynylene, C₁-C₆-alkyloxyphenyl-C₁-C₆-alkylene, phenyloxy-C₁-C₆-alkylene,
C₁-C₆-alkylamino, di(C₁-C₆-alkyl)amino, C₁-C₆-alkylamino-C₁-C₆-alkylene, di(C₁-C₆)-alkylamino-C₁-C₆-alkylene, C₃-C₇-cycloalkyl-(C₀-C₆-alkyl)amino,
C₁-C₆-acyl-(C₀-C₆-alkyl)amido, C₁-C₆-alkylaminocarbonyl, di(C₁-C₆-alkyl)aminocarbonyl, (C₃-C₇-cycloalkyl)aminocarbonyl, di(C₃-C₇-cycloalkyl)aminocarbonyl, C₃-C₇-cycloalkyl-C₁-C₆-alkyleneaminocarbonyl,
C₁-C₆-alkylcarbonyl, C₃-C₇-cycloalkylcarbonyl,
carboxy, carboxamido [—C(O)NH₂], C₁-C₆-alkyloxycarbonyl,
C₁-C₃-alkylsulphanyl, C₁-C₆-alkysulphonyl, C₃-C₇-cycloalkylsulphonyl, C₃-C₇-cycloalkyl-C₁-C₆-alkylenesulphonyl,
C₁-C₆-alkylaminosulphonyl, di(C₁-C₆-alkyl)aminosulphonyl, (C₃-C₇-cycloalkyl)aminosulphonyl, di(C₃-C₇-cycloalkyl)aminosulphonyl, C₃-C₇-cycloalkyl-C₁-C₆-alkyleneaminosulphonyl, C₁-C₆-alkylsulphonylamido, —N(C₀-C₆-alkyl)-C(O)—C₁-C₆-alkyl, —N(C₀-C₆-alkyl)-C(O)—C₃-C₇-cycloalkyl, —N(C₀-C₆-alkyl)-C(O)—N-di(C₀-C₆-alkyl), —N(C₀-C₆-alkyl)-C(O)—O—(C₀-C₆)alkyl, —N(C₀-C₆-alkyl)-C(O)—NH—C₃-C₇-cycloalkyl,
—N(C₀-C₆-alkyl)—SO₂—C₁-C₆-alkyl, —N(C₀-C₆-alkyl)-SO₂—C₃-C₇-cycloalkyl, —N(C₀-C₆-alkyl)-SO₂—N-di(C₀-C₆-alkyl), —N(C₀-C₆-alkyl)-SO₂—NH—(C₃-C₇)-cycloalkyl,
—C(O)—N(H)—C₂-C₆-alkylene-(C₁-C₆-alkyl)amine, —C(O)—N(H)—C₂-C₆-alkylene-[di(C₁-C₆-alkyl)]amine, —C(O)—N(H)—C₂-C₆-alkylene-(C₃-C₇-cycloalkyl)amine, —C(O)—N(H)—C₂-C₆-alkylene-(C₃-C₇-cycloalkyl-C₁-C₆-alkyl)amine,
—S(O₂)—N(H)—C₂-C₆-alkylene-(C₁-C₆-alkyl)amine, —S(O₂)—N(H)—C₂-C₆-alkylene-[di(C₁-C₆-alkyl)]amine, —S(O₂)—N(H)—C₂-C₆-alkylene-(C₃-C₇-cycloalkyl)amine, —S(O₂)—N(H)—C₂-C₆-alkylene-(C₃-C₇-cycloalkyl-C₁-C₆-alkylene)amine,
—O—C₂-C₆-alkylene-(C₁-C₆-alkyl)amine, —O—C₂-C₆-alkylene-[di(C₁-C₆-alkylene)]amine,
or the radicals:

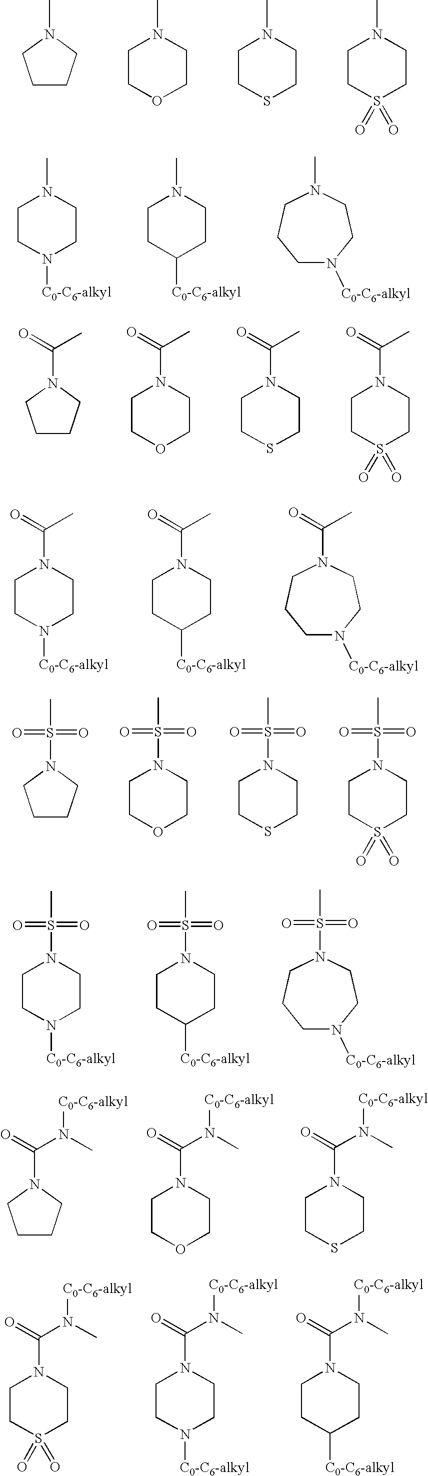

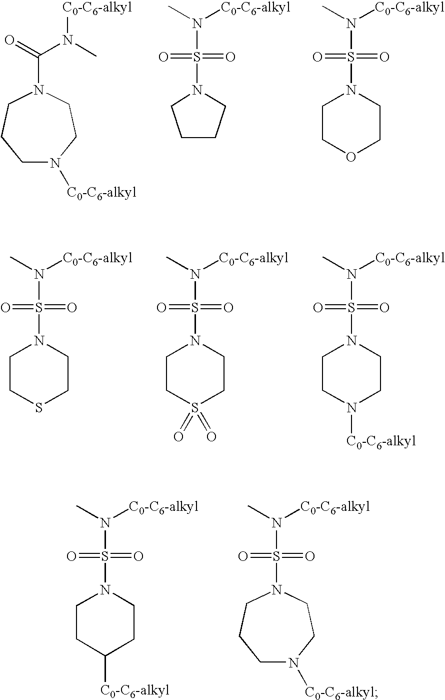

R3 and R4 may together form heterocycloalkyl, cycloalkyl;
Q is an aryl or heteroaryl group
   or the group

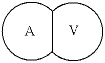

in which
A is a monocyclic aryl or a monocyclic heteroaryl group;
V is a cycloalkylen, cycloalkenylen, heterocycloalkylen or heterocycloalkenylen group;
X is a bond or an ethynyl group;
W is an aryl or heteroaryl group;
Y is selected from the aryl or heteroaryl groups:

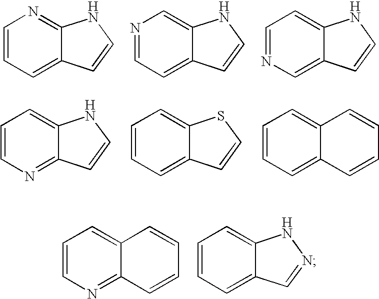

where
R1 substitutes one or more positions of the aryl ring in the radical Y;
R2 substitutes one or more positions of the aryl or heteroaryl ring in the radical Q or in the radical V.

The present invention relates to both possible enantiomeric forms due to the stereocentre of the amino alcohol.

The unbranched C₁-C₆-alkyl groups for the radicals R2 to R5 may be for example a methyl, ethyl, propyl, butyl, pentyl or a hexyl group; and the branched C₃-C₆-alkyl groups for the radicals R1 to R6 may be an isopropyl, isobutyl, sec-butyl, tert-butyl, isopentyl, 2-methylbutyl, 1-methylbutyl, 1-ethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl or a 1,2-dimethylbutyl group.

The branched or unbranched C₂-C₆-alkenyl groups for the radicals R2 to R5 may be for example a vinyl, allyl, (E)-2-methylvinyl, (Z)-2-methylvinyl, homoallyl, (E)-but-2-enyl, (Z)—but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-en-yl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl- or a 1-(1,1-dimethylethyl)ethenyl group.

The C₂-C₆-alkynyl groups for the radicals R2 to R5 may be for example an ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or a 3,3-dimethylbut-1-ynyl group.

The C₁-C₆-alkyloxy groups for the radicals R2 to R5 may be for example a methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)-oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)-oxy, (1,3-dimethylbutyl)oxy or a (1,2-dimethylbutyl)oxy group.

The halogens for the radicals R2 to R5 include fluorine, chlorine, bromine or iodine.

The C₁-C₃-alkylsulphanyl groups for the radicals R3 to R5 may be for example a methylsulphanyl (CH₃S—), ethylsulphanyl (CH₃CH₂S—), propylsulphanyl, isopropylsulphanyl group.

The C₁-C₆-alkylaminocarbonyl groups for the radicals R3 to R5 may be for example a methylaminocarbonyl-, ethylaminocarbonyl-, propylaminocarbonyl-, isopropylaminocarbonyl-, butylaminocarbonyl-, isobutylaminocarbonyl-, sec-butylaminocarbonyl-, tert-butylaminocarbonyl-, pentylaminocarbonyl-, isopentylaminocarbonyl-, (2-methylbutyl)-aminocarbonyl-, (1-methylbutyl)aminocarbonyl-, (1-ethylpropyl)aminocarbonyl-, neopentylaminocarbonyl-, (1,1-dimethylpropyl)aminocarbonyl-, hexylaminocarbonyl-, (4-methylpentyl)aminocarbonyl-, (3-methylpentyl)aminocarbonyl-, (2-methylpentyl)aminocarbonyl-, (1-methylpentyl)aminocarbonyl-, (1-ethylbutyl)aminocarbonyl-, (2-ethylbutyl)-aminocarbonyl-, (3,3-dimethylbutyl)aminocarbonyl-, (2,2-dimethylbutyl)aminocarbonyl-, (1,1-dimethylbutyl)aminocarbonyl-, (2,3-dimethylbutyl)aminocarbonyl-, (1,3-dimethylbutyl)aminocarbonyl- or a (1,2-dimethylbutyl)aminocarbonyl group.

The hydroxy-C₁-C₆-alkylene groups for the radicals R2 to R5 may be a hydroxymethyl (HOCH₂—), 2-hydroxyethyl (HOCH₂CH₂—), 1-hydroxyethyl [CH₃CH(OH)—], 3-hydroxypropyl (HOCH₂CH₂CH₂—), 2-hydroxypropyl [CH₃CH(OH)CH₂—], 1-hydroxypropyl [CH₃CH₂CH(OH)—], 2-hydroxy-1-methylethyl [HOCH₂CH(CH₃)—], 1-hydroxy-1-methylethyl [(CH₃)₂C(OH)—], 4-hydroxybutyl (HOCH₂CH₂CH₂CH₂—), 3-hydroxybutyl [CH₃CH(OH)CH₂CH₂—], 2-hydroxybutyl [CH₃CH₂CH(OH)CH₂—], 1-hydroxybutyl [CH₃CH₂CH₂CH(OH)—], 3-hydroxy-1-methylpropyl [HOCH₂CH₂CH(CH₃)—], 2-hydroxy-1-methylpropyl [CH₃CH(OH)CH(CH₃)—], 1-hydroxy-1-methylpropyl [CH₃CH₂C(CH₃)(OH)—], 1-(hydroxymethyl)propyl [CH₃CH(CH₂OH)—], 3-hydroxy-2-methylpropyl [HOCH₂CH(CH₃)CH₂—], 2-hydroxy-2-methylpropyl [(CH₃)₂C(OH)CH₂—], 1-hydroxy-2-methylpropyl [CH₃CH(CH₃)CH(OH)—] or a 2-hydroxy-1,1-dimethylethyl group [HOCH₂C(CH₃)₂—].

The heterocycloalkyl groups which may form the radicals R3 and R4 together may be for example the following groups:

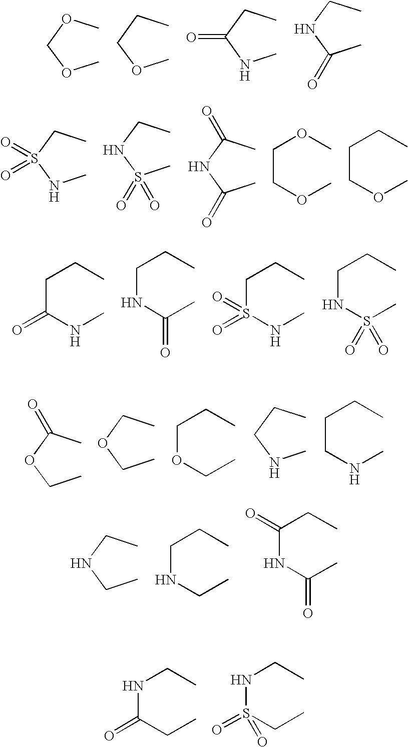

The cycloalkyl groups which may form the radicals R3 and R4 together may be for example the following groups:

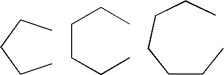

The C₃-C₇-cycloalkyl groups for the radicals R2 to R5 may be for example a cyclopropyl, cyclobutyl, cyclopentyl, cyclohexyl, cycloheptyl group.

The C₃-C₇-heterocycloalkyl groups for the radicals R3 to R5 may be for example a cyclopropyl, cyclobutyl, cyclopentyl, cyclohexyl, cycloheptyl group in which one or two carbon atoms of the ring are replaced independently of one another by an oxygen, nitrogen or sulphur atom.

The monocyclic aryl group for A or Z may be for example a phenyl group which is linked via substitutable positions.

The aryl group for W or Q may be for example a phenyl, naphthyl group which is linked via substitutable positions.

The monocylic heteroaryl group for A or Z may be for example a pyridinyl, pyrimidinyl, furanyl, thienyl, oxazolyl, isoxazolyl, thiazolyl, pyrrolyl, pyrazolyl or an imidazolyl group which is linked via substitutable positions.

The heteroaryl group for W or Q may be for example a pyridinyl, pyrimidinyl, quinolinyl, isoquinolinyl, quinazolinyl, quinoxalinyl, phthalazinyl, 1,5-naphthyridinyl, 1,6-naphthyridinyl, 1,7-naphthyridinyl, 1,8-naphthyridinyl, benzofuranyl, benzothienyl, 1,3-benzodioxolyl, 2,1,3-benzothiadiazolyl, indolyl, indazolyl, furanyl, thienyl, oxazolyl, isoxazolyl, thiazolyl, pyrrolyl, pyrazolyl or an imidazolyl group which is linked via substitutable positions.

The heterocycloalkylen groups for V or Z may be for example the following groups:

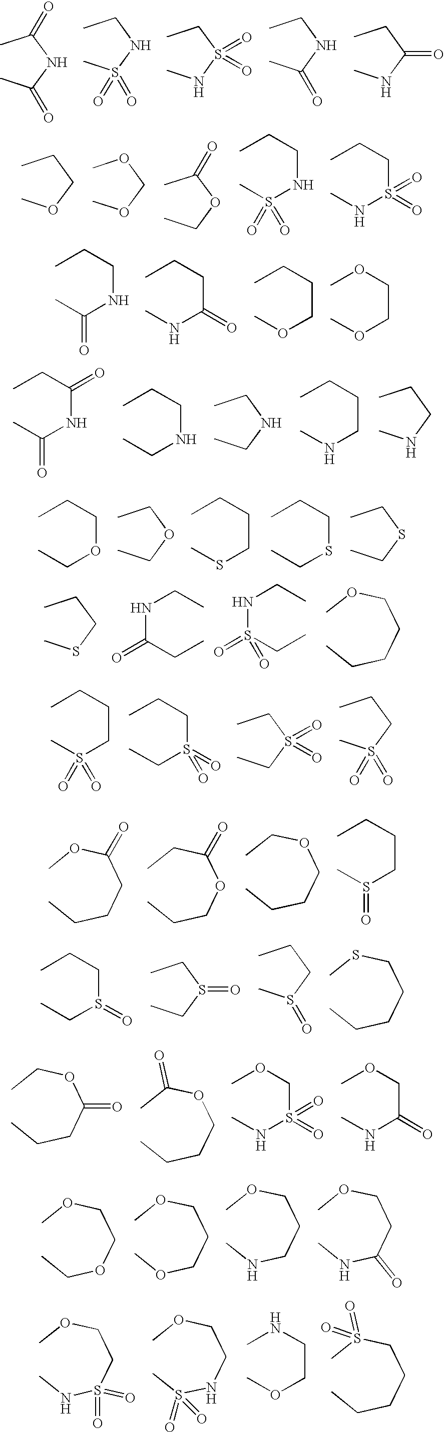

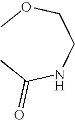

The heterocycloalkenylen groups for V or Z (see formulae III, V) may be for example the following groups:

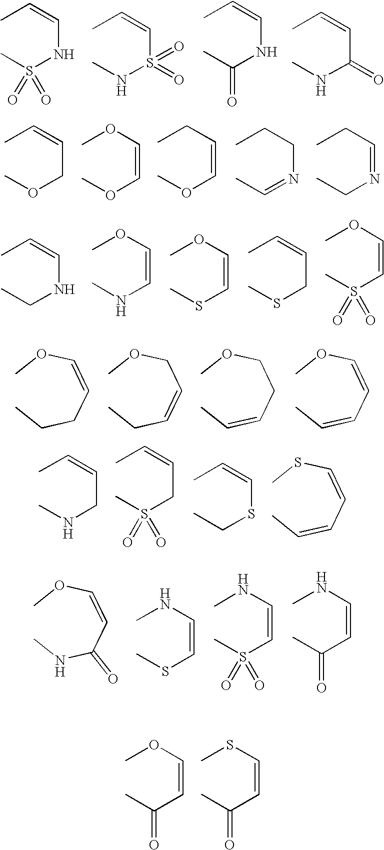

The cycloalkylen groups for V or Z may be for example the following groups:

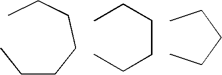

The cycloalkenylen groups for V or Z may be for example the following groups:

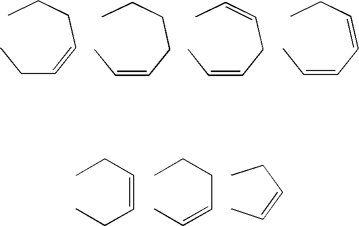

The C₃-C₇-cycloalkyloxy groups for the radicals R2 to R5 may be for example a cyclopropyloxy, cyclobutyloxy, cyclopentyloxy, cyclohexyloxy, cycloheptyloxy group. The C₁-C₆-alkylamino groups for the radicals R2 to R5 may be for example methylamino, ethylamino, propylamino, isopropylamino, butylamino, isobutylamino, sec-butylamino, tert-butylamino, pentylamino, isopentylamino, (2-methylbutyl)amino, (1-methylbutyl)amino, (1-ethylpropyl)amino, neopentylamino, (1,1-dimethylpropyl)amino, hexylamino, (4-methylpentyl)amino, (3-methylpentyl)amino, (2-methylpentyl)amino, (1-methylpentyl)amino, (1-ethylbutyl)amino, (2-ethylbutyl)amino, (3,3-dimethylbutyl)amino, (2,2-dimethylbutyl)amino, (1,1-dimethylbutyl)amino, (2,3-dimethylbutyl)amino, (1,3-dimethylbutyl)amino or a (1,2-dimethylbutyl)amino group.

In the di(C₁-C₆-alkyl)amino groups for the radicals R2 to R5, each of the two radicals on the nitrogen atom of the dialkylamino group may be chosen independently of one another from the following radicals: possible examples are a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the C₃-C₇-cycloalkyl-C₁-C₆-alkyleneoxy groups for the radicals R2 to R5 it is possible to combine each of the C₃-C₇-cycloalkyl groups of the C₃-C₇-cycloalkyl-C₁-C₆-alkyleneoxy group, for example of a cyclopropyl, cyclobutyl, cyclopentyl, cyclohexyl or cycloheptyl group, independently of one another with each C₀-C₆-alkyleneoxy group, for example with a methyleneoxy, ethyleneoxy, propyleneoxy, butyleneoxy, pentyleneoxy, hexyleneoxy group.

In the hydroxy-C₃-C₆-alkenylene groups for the radicals R2 to R5 it is possible for the hydroxy group to be located on any desired position of the C₃-C₆-alkenyl group, for example of an allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-Pent-2-enyl-, (Z)-Pent-2-enyl-, (E)-Pent-1-enyl-, (Z)-Pent-1-enyl-, hex-5-enyl-, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl or a 1-(1,1-dimethylethyl)ethenyl group, and to be combined independently of one another.

In the hydroxy-C₃-C₆-alkynyl groups for the radicals R2 to R5 it is possible for the hydroxy group to be located at any desired position of the C₃-C₆-alkynyl group, for example of a prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or a 3,3-dimethylbut-1-ynyl group.

In the C₁-C₆-alkyloxy-C₃-C₆-alkenylene groups for the radicals R2 to R5 it is possible for the C₁-C₆-alkyloxy group, for example a methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)oxy, (1,3-dimethylbutyl)oxy or a (1,2-dimethylbutyl)oxy group, to be located on any desired position of the C₃-C₆-alkenyl group, for example of an allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl or a 1-(1,1-dimethylethyl)ethenyl group and to be combined independently of one another.

In the C₁-C₆-alkyloxy-C₃-C₆-alkynylene groups for the radicals R2 to R5 it is possible for the C₁-C₆-alkyloxy group, for example a methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)oxy, (1,3-dimethylbutyl)oxy or a (1,2-dimethylbutyl)oxy group, to be located at any desired position of the C₃-C₆-alkynyl group, for example of a prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or a 3,3-dimethylbut-1-ynyl group, and to be combined independently of one another.

In the C₁-C₆-alkyloxyphenyl-C₁-C₆-alkylene groups for the radical R3 to R5 it is possible for the C₁-C₆-alkyloxy group to be selected independently of one another from methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)-oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)-oxy, (1,3-dimethylbutyl)oxy or a (1,2-dimethylbutyl)oxy, and to be combined independently of one another with C₁-C₆-alkylene groups such as, for example, methylene, ethylene, propylene, butylene, pentylene, hexylene.

In the C₃-C₇-cycloalkyl-(C₀-C₆)-alkyleneamino groups of the radicals R3 to R5 it is possible for each of the C₃-C₇-cycloalkyl groups of the C₃-C₇-cycloalkyl-(C₀-C₆)-alkyleneamino group, for example of a cyclopropyl, cyclobutyl, cyclopentyl, cyclohexyl or cycloheptyl group, to be combined independently of one another with each C₀-C₆-alkylene group, for example with a bond, a methylene, ethylene, propylene, butylene, pentylene, hexylene group.

In the C₁-C₆-alkyloxy-C₁-C₆-alkylene groups for the radical R2 to R5, it is possible for the C₁-C₆-alkyloxy group to be selected independently for example from methyloxy, ethyloxy, propyloxy, isopropyloxy, butyloxy, isobutyloxy, sec-butyloxy, tert-butyloxy, pentyloxy, isopentyloxy, (2-methylbutyl)oxy, (1-methylbutyl)oxy, (1-ethylpropyl)oxy, neopentyloxy, (1,1-dimethylpropyl)oxy, hexyloxy, (4-methylpentyl)oxy, (3-methylpentyl)-oxy, (2-methylpentyl)oxy, (1-methylpentyl)oxy, (1-ethylbutyl)oxy, (2-ethylbutyl)oxy, (3,3-dimethylbutyl)oxy, (2,2-dimethylbutyl)oxy, (1,1-dimethylbutyl)oxy, (2,3-dimethylbutyl)-oxy, (1,3-dimethylbutyl)oxy or a (1,2-dimethylbutyl)oxy and to be combined independently of one another with C₁-C₆-alkylene groups such as, for example, methylene, ethylene, propylene, butylene, pentylene, hexylene.

In the di(C₁-C₆-alkyl)amino-C₁-C₆-alkylene group for the radicals R2 to R5 it is possible for each of the two radicals on the nitrogen atom of the amino group to be selected independently for example from methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, and to be combined with C₁-C₆-alkylene groups such as, for example, methylene, ethylene, propylene, butylene, pentylene, hexylene.

The C₃-C₇-cycloalkyl-C₁-C₆-alkylene groups for the radicals R2 to R5 may be for example a cyclopropyloxymethylene, cyclopropyloxyethylene, cyclopropyloxypropylene, cyclopropyloxybutylene, cyclopropyloxypentylene, cyclopropyloxyhexylene, cyclobutyloxymethylene, cyclobutyloxyethylene, cyclobutyloxypropylene, cyclobutyloxybutylene, cyclobutyloxypentylene, cyclobutyloxyhexylene, cyclopentyloxymethylene, cyclopentyloxyethylene, cyclopentyloxypropylene, cyclopentyloxybutylene, cyclopentyloxypentylene, cyclopentyloxyhexylene, cyclohexyloxymethylene, cyclohexyloxyethylene, cyclohexyloxypropylene, cyclohexyloxybutylene, cyclohexyloxypentylene, cyclohexyloxyhexylene, cycloheptyloxymethylene, cycloheptyloxyethylene, cycloheptyloxypropylene, cycloheptyloxybutylene, cycloheptyloxypentylene, cycloheptyloxyhexylen group.

In the C₁-C₆-alkylamino-C₁-C₆-alkylene groups for the radicals R2 to R5 it is possible for the C₁-C₆-alkylamino group to be selected independently for example from methylamino, ethylamino, propylamino, isopropylamino, butylamino, isobutylamino, sec-butylamino, tert-butylamino, pentylamino, isopentylamino, (2-methylbutyl)amino, (1-methylbutyl)amino, (1-ethylpropyl)amino, neopentylamino, (1,1-dimethylpropyl)amino, hexylamino, (4-methylpentyl)amino, (3-methylpentyl)amino, (2-methylpentyl)amino, (1-methylpentyl)amino, (1-ethylbutyl)amino, (2-ethylbutyl)amino, (3,3-dimethylbutyl)amino, (2,2-dimethylbutyl)amino, (1,1-dimethylbutyl)amino, (2,3-dimethylbutyl)amino, (1,3-dimethylbutyl)amino or a (1,2-dimethylbutyl)amino and to be combined with C₁-C₆-alkylene groups such as, for example, methylene, ethylene, propylene, butylene, pentylene, hexylene.

The phenyloxy-C₁-C₆-alkylene groups for the radicals R3 to R5 may be for example a phenyloxymethyl, phenyloxyethyl, phenyloxypropyl, phenyloxybutyl, phenyloxypentyl, phenyloxyhexyl group.

In the C₁-C₆-acyl-(C₀-C₆-alkyl)amido groups for the radicals R3 to R5, it is possible for each of the C₁-C₆-acyl groups, for example a formyl, acetyl, propionyl, 2-methylpropionyl, 2,2-dimethylpropionyl, butyryl, 2-methylbutyryl, 3-methylbutyryl, 2,2-dimethylbutyryl, 2-ethylbutyryl, pentanoyl, 2-methylpentanoyl, 3-methylpentanoyl, 4-methylpentanoyl or a hexanoyl group, to be combined independently of one another with each (C₀-C₆-alkyl)amido group, for example a hydrogen atom, a methylamido, ethylamido, propylamido, isopropylamido, butylamido, isobutylamido, sec-butylamido, tert-butylamido, pentylamido, isopentylamido, (2-methylbutyl)amido, (1-methylbutyl)amido, (1-ethylpropyl)amido, neopentylamido, (1,1-dimethylpropyl)amido, hexylamido, (4-methylpentyl)amido, (3-methylpentyl)amido, (2-methylpentyl)amido, (1-methylpentyl)amido, (1-ethylbutyl)amido, (2-ethylbutyl)amido, (3,3-dimethylbutyl)amido, (2,2-dimethylbutyl)-amido, (1,1-dimethylbutyl)amido, (2,3-dimethylbutyl)amido, (1,3-dimethylbutyl)amido or a (1,2-dimethylbutyl)amido group.

The C₁-C₆-alkylaminocarbonyl groups for the radicals R3 to R5 may be for example a methylaminocarbonyl, ethylaminocarbonyl, propylaminocarbonyl, isopropylaminocarbonyl, butylaminocarbonyl, isobutylaminocarbonyl, sec-butylaminocarbonyl, tert-butylaminocarbonyl, pentylaminocarbonyl, isopentylaminocarbonyl, (2-methylbutyl)-aminocarbonyl, (1-methylbutyl)aminocarbonyl, (1-ethylpropyl)aminocarbonyl, neopentylaminocarbonyl, (1,1-dimethylpropyl)aminocarbonyl, hexylaminocarbonyl, (4-methylpentyl)aminocarbonyl, (3-methylpentyl)aminocarbonyl, (2-methylpentyl)aminocarbonyl, (1-methylpentyl)aminocarbonyl, (1-ethylbutyl)aminocarbonyl, (2-ethylbutyl)aminocarbonyl, (3,3-dimethylbutyl)aminocarbonyl, (2,2-dimethylbutyl)aminocarbonyl, (1,1-dimethylbutyl)aminocarbonyl, (2,3-dimethylbutyl)aminocarbonyl, (1,3-dimethylbutyl)-aminocarbonyl or a (1,2-dimethylbutyl)aminocarbonyl group.

In the di(C₁-C₆-alkyl)aminocarbonyl groups for the radicals R3 to R5, each of the two C₁-C₆-alkyl radicals on the nitrogen atom of the di(C₁-C₆-alkyl)aminocarbonyl group may be independently of one another for example a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

The (C₃-C₇-cycloalkyl)aminocarbonyl groups for the radicals R3 to R5 may be for example a cyclopropylaminocarbonyl, cyclobutylaminocarbonyl, cyclopentylaminocarbonyl, cyclohexylaminocarbonyl or cycloheptylaminocarbonyl group.

In the di(C₃-C₇-cycloalkyl)aminocarbonyl groups for the radicals R3 to R5, each of the two C₃-C₇-cycloalkyl radicals on the nitrogen atom of the di(C₃-C₇-cycloalkyl)aminocarbonyl group may be independently of one another for example a cyclopropyl, cyclobutyl, cyclopentyl, cyclohexyl or cycloheptyl group.

In the C₃-C₇-cycloalkyl-C₁-C₆-alkyleneaminocarbonyl groups of the radicals R3 to R5 it is possible for each of the C₃-C₇-cycloalkyl groups of the C₃-C₇-cycloalkyl-C₁-C₆-alkyleneaminocarbonyl groups, for example of a cyclopropyl, cyclobutyl, cyclopentyl, cyclohexyl or cycloheptyl group, to be combined independently of one another with each C₁-C₆-alkyleneaminocarbonyl group, for example with a methyleneaminocarbonyl, ethyleneaminocarbonyl, propyleneaminocarbonyl, butyleneaminocarbonyl, pentyleneaminocarbonyl, hexyleneaminocarbonyl group.

The C₁-C₆-alkylcarbonyl groups for the radicals R3 to R5 may be for example a methylcarbonyl, ethylcarbonyl, propylcarbonyl, isopropylcarbonyl, butylcarbonyl, isobutylcarbonyl, sec-butylcarbonyl, tert-butylcarbonyl, pentylcarbonyl, isopentylcarbonyl, (2-methylbutyl)carbonyl, (1-methylbutyl)carbonyl, (1-ethylpropyl)carbonyl, neopentylcarbonyl, (1,1-dimethylpropyl)carbonyl, hexylcarbonyl, (4-methylpentyl)carbonyl, (3-methylpentyl)carbonyl, (2-methylpentyl)carbonyl, (1-methylpentyl)carbonyl, (1-ethylbutyl)-carbonyl, (2-ethylbutyl)carbonyl, (3,3-dimethylbutyl)carbonyl, (2,2-dimethylbutyl)carbonyl, (1,1-dimethylbutyl)carbonyl, (2,3-dimethylbutyl)carbonyl, (1,3-dimethylbutyl)carbonyl or a (1,2-dimethylbutyl)carbonyl group.

The C₃-C₇-cycloalkylcarbonyl groups for the radicals R3 to R5 may be for example a cyclopropylcarbonyl, cyclobutylcarbonyl, cyclopentylcarbonyl, cyclohexylcarbonyl or cycloheptylcarbonyl group.

The C₁-C₆-alkyloxycarbonyl groups for the radicals R3 to R5 may be for example a methyloxycarbonyl, ethyloxycarbonyl, propyloxycarbonyl, isopropyloxycarbonyl, butyloxycarbonyl, isobutyloxycarbonyl, sec-butyloxycarbonyl, tert-butyloxycarbonyl, pentyloxycarbonyl, isopentyloxycarbonyl, (2-methylbutyl)oxycarbonyl, (1-methylbutyl)oxycarbonyl, (1-ethylpropyl)oxycarbonyl, neopentyloxycarbonyl, (1,1-dimethylpropyl)oxycarbonyl, hexyloxycarbonyl, (4-methylpentyl)oxycarbonyl, (3-methylpentyl)oxycarbonyl, (2-methylpentyl)oxycarbonyl, (1-methylpentyl)oxycarbonyl, (1-ethylbutyl)oxycarbonyl, (2-ethylbutyl)oxycarbonyl, (3,3-dimethylbutyl)oxycarbonyl, (2,2-dimethylbutyl)oxycarbonyl, (1,1-dimethylbutyl)oxycarbonyl, (2,3-dimethylbutyl)oxycarbonyl, (1,3-dimethylbutyl)oxycarbonyl or a (1,2-dimethylbutyl)oxycarbonyl group.

The C₁-C₆-alkylsulphonyl groups for the radicals R3 to R5 may be for example a methylsulphonyl, ethylsulphonyl, propylsulphonyl, isopropylsulphonyl, butylsulphonyl, isobutylsulphonyl, sec-butylsulphonyl, tert-butylsulphonyl, pentylsulphonyl, isopentylsulphonyl, (2-methylbutyl)sulphonyl, (1-methylbutyl)sulphonyl, (1-ethylpropyl)sulphonyl, neopentylsulphonyl, (1,1-dimethylpropyl)sulphonyl, hexylsulphonyl, (4-methylpentyl)-sulphonyl, (3-methylpentyl)sulphonyl, (2-methylpentyl)sulphonyl, (1-methylpentyl)-sulphonyl, (1-ethylbutyl)sulphonyl, (2-ethylbutyl)sulphonyl, (3,3-dimethylbutyl)sulphonyl, (2,2-dimethylbutyl)sulphonyl, (1,1-dimethylbutyl)sulphonyl, (2,3-dimethylbutyl)sulphonyl, (1,3-dimethylbutyl)sulphonyl or a (1,2-dimethylbutyl)sulphonyl group.

The C₃-C₇-cycloalkylsulphonyl groups for the radicals R3 to R5 may be for example a cyclopropylsulphonyl, cyclobutylsulphonyl, cyclopentylsulphonyl, cyclohexylsulphonyl or cycloheptylsulphonyl group.

In the C₃-C₇-cycloalkyl-C₁-C₆-alkylenesulphonyl groups of the radicals R3 to R5 it is possible for each of the C₃-C₇-cycloalkyl groups of the C₃-C₇-cycloalkyl-C₁-C₆-alkylenesulphonyl groups, for example of a cyclopropyl, cyclobutyl, cyclopentyl, cyclohexyl or cycloheptyl group, to be combined independently of one another with each C₁-C₆-alkylenesulphonyl group, for example with a methylenesulphonyl, ethylenesulphonyl, propylenesulphonyl, butylenesulphonyl, pentylenesulphonyl, hexylenesulphonyl group.

The C₁-C₆-alkylaminosulphonyl groups for the radicals R3 to R5 may be for example a methylaminosulphonyl, ethylaminosulphonyl, propylaminosulphonyl, isopropylaminosulphonyl, butylaminosulphonyl, isobutylaminosulphonyl, sec-butylaminosulphonyl, tert-butylaminosulphonyl, pentylaminosulphonyl, isopentylaminosulphonyl, (2-methylbutyl)aminosulphonyl, (1-methylbutyl)aminosulphonyl, (1-ethylpropyl)aminosulphonyl, neopentylaminosulphonyl, (1,1-dimethylpropyl)aminosulphonyl, hexylaminosulphonyl, (4-methylpentyl)aminosulphonyl, (3-methylpentyl)aminosulphonyl, (2-methylpentyl)-aminosulphonyl, (1-methylpentyl)aminosulphonyl, (1-ethylbutyl)aminosulphonyl, (2-ethylbutyl)aminosulphonyl, (3,3-dimethylbutyl)aminosulphonyl, (2,2-dimethylbutyl)-aminosulphonyl, (1,1-dimethylbutyl)aminosulphonyl, (2,3-dimethylbutyl)aminosulphonyl, (1,3-dimethylbutyl)aminosulphonyl or a (1,2-dimethylbutyl)aminosulphonyl group.

In the di(C₁-C₆-alkyl)aminosulphonyl groups for the radicals R4 to R6, each of the two C₁-C₆-alkyl radicals on the nitrogen atom of the di(C₁-C₆-alkyl)aminosulphonyl group may be independently of one another for example a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

The (C₃-C₇-cycloalkyl)aminosulphonyl groups for the radicals R3 to R5 may be for example a cyclopropylaminosulphonyl, cyclobutylaminosulphonyl, cyclopentylaminosulphonyl, cyclohexylaminosulphonyl or cycloheptylaminosulphonyl group.

In the di(C₃-C₇-cycloalkyl)aminosulphonyl groups for the radicals R3 to R5, each of the two C₃-C₇-cycloalkyl radicals on the nitrogen atom of the di(C₃-C₇-cycloalkyl)aminosulphonyl group may be independently of one another for example a cyclopropyl, cyclobutyl, cyclopentyl, cyclohexyl or cycloheptyl group.

In the C₃-C₇-cycloalkyl-C₁-C₆-alkyleneaminosulphonyl groups of the radicals R3 to R5, each of the C₃-C₇-cycloalkyl groups of the C₃-C₇-cycloalkyl-C₁-C₆-alkyleneaminosulphonyl groups, for example of a cyclopropyl, cyclobutyl, cyclopentyl, cyclohexyl or cycloheptyl group, can be combined independently of one another with each C₁-C₆-alkyleneaminosulphonyl group, for example with a methyleneaminosulphonyl, ethyleneaminosulphonyl, propyleneaminosulphonyl, butyleneaminosulphonyl, pentyleneaminosulphonyl, hexyleneaminosulphonyl group.

The C₁-C₆-alkylsulphonylamido groups for the radicals R3 to R5 may be for example a methylsulphonylamido, ethylsulphonylamido, propylsulphonylamido, isopropylsulphonylamido, butylsulphonylamido, isobutylsulphonylamido, sec-butylsulphonylamido, tert-butylsulphonylamido, pentylsulphonylamido, isopentylsulphonylamido, (2-methylbutyl)-sulphonylamido, (1-methylbutyl)sulphonylamido, (1-ethylpropyl)sulphonylamido, neopentylsulphonylamido, (1,1-dimethylpropyl)sulphonylamido, hexylsulphonylamido, (4-methylpentyl)sulphonylamido, (3-methylpentyl)sulphonylamido, (2-methylpentyl)-sulphonylamido, (1-methylpentyl)sulphonylamido, (1-ethylbutyl)sulphonylamido, (2-ethylbutyl)sulphonylamido, (3,3-dimethylbutyl)sulphonylamido, (2,2-dimethylbutyl)-sulphonylamido, (1,1-dimethylbutyl)sulphonylamido, (2,3-dimethylbutyl)sulphonylamido, (1,3-dimethylbutyl)sulphonylamido or a (1,2-dimethylbutyl)sulphonylamido group.

In the —N(C₀-C₆-alkyl)-C(O)—C₁-C₆-alkyl groups of the radicals R3 to R5, each of the (C₀-C₆-alkyl) groups on the nitrogen atom of the —N(C₀-C₆-alkyl)-C(O)—C₁-C₆-alkyl groups, for example a hydrogen, a methyl, ethyl, propyl, isopropyl, butyl, isobutyl, secbutyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may be combined independently of one another with each C₁-C₆-alkyl group on the carbonyl group of the amide, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N—(C₀-C₆-alkyl)-C(O)—C₃-C₇-cycloalkyl groups of the radicals R3 to R5, each of the (C₀-C₆-alkyl) groups on the nitrogen atom of the —N(C₀-C₆-alkyl)-C(O)—C₁-C₆-alkyl groups, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may be combined independently of one another with each C₃-C₇-cycloalkyl group on the carbonyl group of the amide, for example with a cyclopropyl, cyclobutyl, cyclopentyl, cyclohexyl or cycloheptyl group.

In the —N(C₀-C₆-alkyl)-C(O)—N-di(C₀-C₆-alkyl) groups of the radicals R3 to R5, all three (C₀-C₆-alkyl) groups may be independently of one another a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N(C₀-C₆-alkyl)-C(O)—O—(C₀-C₆-alkyl) groups of the radicals R3 to R5, both (C₀-C₆-alkyl) groups may be independently of one another a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N(C₀-C₆-alkyl)-C(O)—NH—(C₃-C₇-cycloalkyl) groups of the radicals R3 to R5, each of the (C₀-C₆-alkyl) groups on the nitrogen atom of the —N(C₀-C₆-alkyl)-C(O)—NH—(C₃-C₇-cycloalkyl) groups, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may independently of one another be combined with each C₃-C₇-cycloalkyl group on the terminal nitrogen atom of the urea, for example with a cyclopropyl, cyclobutyl, cyclopentyl, cyclohexyl or cycloheptyl group.

In the —N(C₀-C₆-alkyl)-SO₂—(C₁-C₆-alkyl) groups of the radicals R3 to R5, each of the (C₀-C₆-alkyl) groups on the nitrogen atom of the —N(C₀-C₆-alkyl)-SO₂—(C₁-C₆-alkyl) group, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may independently of one another be combined with each C₁-C₆-alkyl group on the sulphonyl group of the sulphonamide, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N(C₀-C₆-alkyl)-SO₂—C₃-C₇-cycloalkyl groups of the radicals R3 to R5, each of the (C₀-C₆-alkyl) groups on the nitrogen atom of the —N(C₀-C₆-alkyl)-SO₂—C₃-C₇-cycloalkyl group, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may be combined independently of one another with each C₃-C₇-cycloalkyl group on the sulphonyl group, for example with a cyclopropyl, cyclobutyl, cyclopentyl, cyclohexyl or cycloheptyl group.

In the —N(C₀-C₆-alkyl)-SO₂—N-di(C₀-C₆-alkyl) groups of the radicals R3 to R5, all three (C₀-C₆-alkyl) groups may be independently of one another a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —N(C₀-C₆-alkyl)-SO₂—NH—(C₃-C₇)-cycloalkyl groups of the radicals R3 to R5, the C₀-C₆-alkyl group of the —N(C₀-C₆-alkyl)-SO₂—NH—(C₃-C₇)-cycloalkyl group, for example a hydrogen, a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group, may be combined independently of one another with each C₃-C₇-cycloalkyl group, for example with a cyclopropyl, cyclobutyl, cyclopentyl, cyclohexyl or cycloheptyl group.

In the —C(O)—N(H)—C₂-C₆-alkylene-(C₁-C₆-alkyl)amine groups of the radicals R3 to R5, each of the C₂-C₆-alkylene groups on the nitrogen atom of the —C(O)—N(H)—C₂-C₆-alkylene-(C₁-C₆-alkyl)amine group, for example an ethylene, propylene, butylene, pentylene or hexylene group, may be combined independently of one another with each C₁-C₆-alkyl group on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —C(O)—N(H)—C₂-C₆-alkylene-[di(C₁-C₆-alkyl)]amine groups of the radicals R3 to R5, each of the C₂-C₆-alkylene groups on the nitrogen atom of the —C(O)—N(H)—C₂-C₆-alkylene-[di(C₁-C₆-alkyl)]amine group, for example an ethylene, propylene, butylene, pentylene or hexylene group, may be combined independently of one another with each of the two identically or different C₁-C₆-alkyl groups on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl-, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —C(O)—N(H)—C₂-C₆-alkylene-(C₃-C₇-cycloalkyl)amine groups of the radicals R3 to R5, each of the (C₂-C₆-alkylene) groups of the —C(O)—N(H)—C₂-C₆-alkylene-(C₃-C₇-cycloalkyl)amine group, for example an ethylene, propylene, butylene, pentylene or hexylene group, may be combined independently of one another with each C₃-C₇-cycloalkyl group on the amine, for example with a cyclopropyl, cyclobutyl, cyclopentyl, cyclohexyl or cycloheptyl group.

In the —C(O)—N(H)—C₂-C₆-alkylene-(C₃-C₇-cycloalkyl-C₁-C₆-alkylene)amine groups of the radicals R3 to R5, each of the (C₂-C₆-alkylene) groups of the —C(O)—N(H)—C2-C6-alkylene-(C3-C6-cycloalkyl-C1-C6-alkylene)amine group, for example an ethylene, propylene, butylene, pentylene or hexylene group, may be combined independently of one another with each C₃-C₇-cycloalkyl-C₁-C₆-alkylene group on the amine, for example with a cyclopropylmethylene, cyclopropylethylene, cyclopropylpropylene, cyclopropylbutylene, cyclopropylpentylene, cyclopropylhexylene, cyclobutylmethylene, cyclobutylethylene, cyclobutylpropylene, cyclobutylbutylene, cyclobutylpentylene, cyclobutylhexylene, cyclopentylmethylene, cyclopentylethylene, cyclopentylpropylene, cyclopentylhexylene, cyclohexylmethylene, cyclohexylethylene, cyclohexylpropylene, cyclohexylbutylene, cyclohexylpentylene, cyclohexylhexylene, cycloheptylmethylene, cycloheptylethylene, cycloheptylpropylene, cycloheptylbutylene, cycloheptylpentylene or cycloheptylhexylene group.

In the —S(O₂)—N(H)—C₂-C₆-alkylene-(C₁-C₆-alkyl)amine groups of the radicals R3 to R5, the (C₂-C₆-alkylene) groups of the —S(O₂)—N(H)—C₂-C₆-alkylene-(C₁-C₆-alkyl)amine group, for example an ethylene, propylene, butylene, pentylene or hexylene group, may be combined independently of one another with each C₁-C₆-alkyl group on the amino group, for example with a methyl, ethyl, propyl, isopropyl, butyl, isobutyl, sec-butyl, tertbutyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —S(O₂)—N(H)—C₂-C₆-alkylene-[di(C₁-C₆-alkyl)]amine groups of the radicals R3 to R5, the C₂-C₆-alkylene group of the —S(O₂)—N(H)—C₂-C₆-alkylene-[di(C₁-C₆-alkyl)]amine group, for example an ethylene, propylene, butylene, pentylene or hexylene group, may be combined independently of one another with each of the two C₁-C₆-alkyl groups on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl-, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —S(O₂)—N(H)—C₂-C₆-alkylene-(C₃-C₇-cycloalkyl)amine groups of the radicals R3 to R5, the C₂-C₆-alkylene group of the —S(O₂)—N(H)—C₂-C₆-alkylene-(C₃-C₇-cycloalkyl)-amine group, for example an ethylene, propylene, butylene, pentylene or hexylene group, may be combined independently of one another with each C₃-C₇-cycloalkyl group on the amino group, for example with a cyclopropyl, cyclobutyl, cyclopentyl, cyclohexyl or cycloheptyl group.

In the —S(O₂)—N(H)—C₂-C₆-alkylene-(C₃-C₇-cycloalkyl-C₁-C₆-alkylene)amine groups of the radicals R3 to R5, each C₂-C₆-alkylene group of the —S(O₂)—N(H)—C₂-C₆-alkylene-(C₃-C₇-cycloalkyl-C₁-C₆-alkylene)amine group, for example an ethylene, propylene, butylene, pentylene or hexylene group, may be combined independently of one another with each C₃-C₇-cycloalkyl-C₁-C₆-alkylene group on the amine, for example with a cyclopropylmethylene, cyclopropylethylene, cyclopropylpropylene, cyclopropylbutylene, cyclopropylpentylene, cyclopropylhexylene, cyclobutylmethylene, cyclobutylethylene, cyclobutylpropylene, cyclobutylbutylene, cyclobutylpentylene, cyclobutylhexylene, cyclopentylmethylene, cyclopentylethylene, cyclopentylpropylene, cyclopentylhexylene, cyclohexylmethylene, cyclohexylethylene, cyclohexylpropylene, cyclohexylbutylene, cyclohexylpentylene, cyclohexylhexylene, cycloheptylmethylene, cycloheptylethylene, cycloheptylpropylene, cycloheptylbutylene, cycloheptylpentylene or cycloheptylhexylene group.

In the —O—C₂-C₆-alkylene-(C₁-C₆-alkyl)amine groups of the radicals R3 to R5, the C₂-C₆-alkylene group of the —O—C₂-C₆-alkylene-(C₁-C₆-alkyl)amine group, for example an ethylene, propylene, butylene, pentylene or hexylene group, may be combined independently of one another with each C₁-C₆-alkyl group on the amino group, for example a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

In the —O—C₂-C₆-alkylene-[di(C₁-C₆-alkyl)]amine groups of the radicals R3 to R5, the C₂-C₆-alkylene group of the —O—C₂-C₆-alkylene-[di(C₁-C₆-alkyl)]amine group, for example an ethylene, propylene, butylene, pentylene or hexylene group, may be combined independently of one another with two freely selectable C₁-C₆-alkyl groups on the amino group, for example with a methyl, ethyl, propyl, isopropyl, butyl, isobutyl, sec-butyl, tert-butyl, pentyl, isopentyl, (2-methylbutyl), (1-methylbutyl), (1-ethylpropyl), neopentyl, (1,1-dimethylpropyl), hexyl-, (4-methylpentyl), (3-methylpentyl), (2-methylpentyl), (1-methylpentyl), (1-ethylbutyl), (2-ethylbutyl), (3,3-dimethylbutyl), (2,2-dimethylbutyl), (1,1-dimethylbutyl), (2,3-dimethylbutyl), (1,3-dimethylbutyl) or a (1,2-dimethylbutyl) group.

Compounds preferred according to the present invention are those of the formula I in which

Y is selected from the heteroaryl groups:

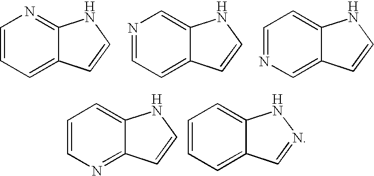

Compounds more preferred according to the present invention are those of the formula II

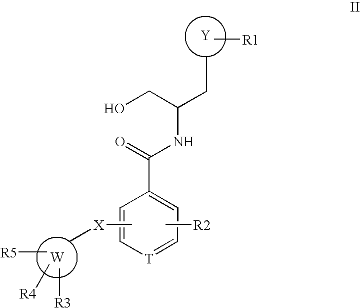

wherein
T is a nitrogen atom or a CH group;
Y is selected from the heteroaryl groups:

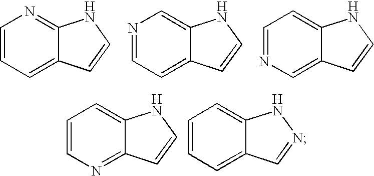

and
R1, R2, R3, R4, R5, X and W have the same meaning as defined in formula I.

Compounds likewise more preferred according to the present invention are those of the formula III

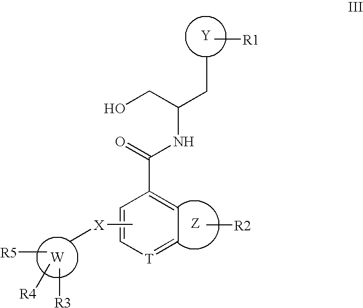

wherein
T is a nitrogen atom or a CH group;
Z is a monocyclic aryl or a monocyclic heteroaryl group or a cycloalkylen, cycloalkenylen, heterocycloalkylen or heterocycloalkenylen group;
Y is selected from the heteroaryl groups:

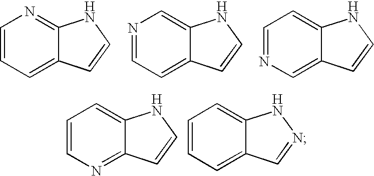

and
R1, R2, R3, R4, R5, X and W have the same meaning as defined in formula I.

Compounds particularly preferred according to the present invention are those of the formula IV

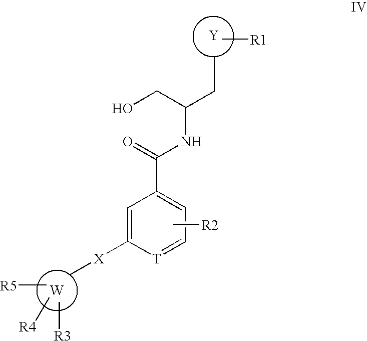

wherein
T is a nitrogen atom or a CH group;
Y is selected from the heteroaryl groups

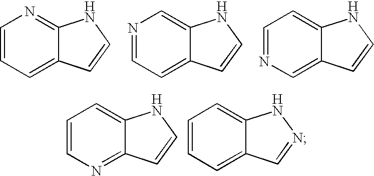

and
R1, R2, R3, R4, R5, X, Y and W have the same meaning as defined in formula I.

Compounds likewise particularly preferred according to the present invention are those of the formula V

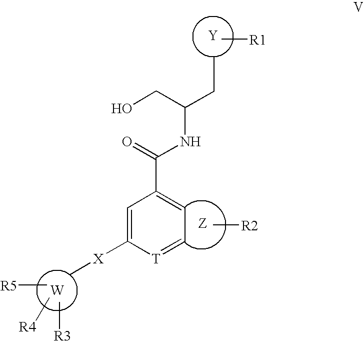

wherein
T is a nitrogen atom or a CH group;
Z is a monocyclic aryl or a monocyclic heteroaryl group or a cycloalkylen, cycloalkenylen, heterocycloalkylen or heterocycloalkenylen group;
Y is selected from the heteroaryl groups:

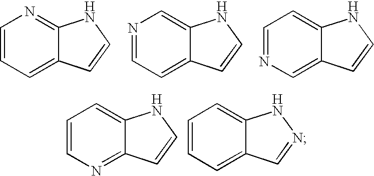

and
R1, R2, R3, R4, R5, X, Y and W have the same meaning as defined in formula I.

The following compounds are most particularly preferred:
1 5′-Fluoro-4-isopropoxy-biphenyl-3,3′-dicarboxylic acid 3-{[2-hydroxy-1-(1H-pyrrolo[2,3-b]pyridin-3-ylmethyl)-ethyl]-amide}3′-methylamide;
2 3′-Chloro-4-isopropoxy-biphenyl-3,4′-dicarboxylic acid 3-{[2-hydroxy-1-(1H-pyrrolo[2,3-b]pyridin-3-ylmethyl)-ethyl]-amide}4′-methylamide;
3 N-[1-Hydroxymethyl-2-(1H-pyrrolo[2,3-b]pyridin-3-yl)-ethyl]-2-isopropoxy-5-(2-methoxy-phenylethynyl)-benzamide;
4 N-[1-Hydroxymethyl-2-(1H-pyrrolo[2,3-b]pyridin-3-yl)-ethyl]-2-isopropoxy-5-phenylethynyl-benzamide;
5 6-(3-Chloro-4-methylcarbamoyl-phenyl)-2,2-dimethyl-2H-chromene-8-carboxylic acid [2-hydroxy-1-(1H-pyrrolo[2,3-b]pyridin-3-ylmethyl)-ethyl]-amide;
6 6-Methoxy-2-(3,4,5-trimethoxy-phenyl)-quinoline-4-carboxylic acid ((R)-2-benzo[b]-thiophen-3-yl-1-hydroxy-methyl-ethyl)-amide;
7 4-Ethoxy-3′-methoxy-biphenyl-3-carboxylic acid ((R)-2-benzo[b]thiophen-3-yl-1-hydroxymethyl-ethyl)-amide;
8 6-Methoxy-2-(3,4,5-trimethoxy-phenyl)-quinoline-4-carboxylic acid (1-hydroxymethyl-2-quinolin-3-yl-ethyl)-amide;
9 6-Methoxy-2-(3,4,5-trimethoxy-phenyl)-quinoline-4-carboxylic acid ((R)-2-hydroxy-1-naphthalen-2-ylmethyl-ethyl)-amide;
10 6-Iodo-2-(3,4,5-trimethoxy-phenyl)-quinoline-4-carboxylic acid ((R)-2-hydroxy-1-naphthalen-2-ylmethyl-ethyl)-amide;
11 6-Methoxy-2-(3,4,5-trimethoxy-phenyl)-quinoline-4-carboxylic acid (2-hydroxy-1-naphthalen-1-ylmethyl-ethyl)-amide;
12 6-Methoxy-2-(3,4,5-trimethoxy-phenyl)-quinoline-4-carboxylic acid (2-hydroxy-1-naphthalen-2-ylmethyl-ethyl)-amide;
13 3′-Chloro-4-trifluoromethoxy-biphenyl-3,4′-dicarboxylic acid 3-{[2-hydroxy-1-(4-fluoro-1H-indazol-3-ylmethyl)-ethyl]-amide}4′-methylamide;
14 3′-Chloro-4-trifluoromethoxy-biphenyl-3,4′-dicarboxylic acid 3-{[2-hydroxy-1-(7-fluoro-1H-indazol-3-ylmethyl)-ethyl]-amide}4′-methylamide;
15 3′-Chloro-4-trifluoromethoxy-biphenyl-3,4′-dicarboxylic acid 3-{[2-hydroxy-1-(7-trifluoromethyl-1H-indazol-3-ylmethyl)-ethyl]-amide}4′-methylamide;
16 N-[2-hydroxy-1-(4-fluoro-1H-indazol-3-ylmethyl)-ethyl]-2-trifluoromethoxy-5-[(4-methylaminocarbonyl-phenyl)-ethynyl]-benzamide;
17 N-[2-hydroxy-1-(7-fluoro-1H-indazol-3-ylmethyl)-ethyl]-2-trifluoromethoxy-5-[(4-methylaminocarbonyl-phenyl)-ethynyl]-benzamide;
18 N-[2-hydroxy-1-(7-trifluoromethyl-1H-indazol-3-ylmethyl)-ethyl]-2-trifluoromethoxy-5-[(4-methylaminocarbonyl-phenyl)-ethynyl]-benzamide;
19 6-Bromo-2-(3,4,5-trimethoxy-phenyl)-quinoline-4-carboxylic acid [2-hydroxy-1-(1H-pyrrolo[3,2-b]pyridin-3-ylmethyl)-ethyl]-amide;
20 2-(7-Methoxy-benzofuran-2-yl)-6-trifluoromethoxy-quinoline-4-carboxylic acid [2-hydroxy-1-(1H-pyrrolo[3,2-b]pyridin-3-ylmethyl)-ethyl]-amide;
21 2-(3-Fluoro-4-methoxy-phenyl)-6-methoxy-quinazoline-4-carboxylic acid [2-hydroxy-1-(1H-pyrrolo[3,2-b]pyridin-3-ylmethyl)-ethyl]-amide;
22 N-[1-Hydroxymethyl-2-(1H-pyrrolo[3,2-b]pyridin-3-yl)-ethyl]-2-isopropoxy-5-(2-methoxy-phenylethynyl)-benzamide;
23 N-[1-Hydroxymethyl-2-(1H-pyrrolo[3,2-b]pyridin-3-yl)-ethyl]-2-isopropoxy-5-phenylethynyl-benzamide;
24 5′-Fluoro-4-isopropoxy-biphenyl-3,3′-dicarboxylic acid 3-{[1-hydroxymethyl-2-(1H-pyrrolo[3,2-b]pyridin-2-yl)-ethyl]-amide}3′-methylamide;
25 3′-Chloro-4-isopropoxy-biphenyl-3,4′-dicarboxylic acid 3-{[1-hydroxymethyl-2-(1H-pyrrolo[3,2-b]pyridin-3-yl)-ethyl]-amide}4′-methylamide;
26 7-(3,4-Dimethoxy-phenyl)-2,3,4,5-tetrahydro-benzo[b]oxepine-9-carboxylic acid [1-hydroxymethyl-2-(1H-pyrrolo[3,2-b]pyridin-3-yl)-ethyl]-amide
27 N-[1-Hydroxymethyl-2-(1H-pyrrolo[3,2-c]pyridin-3-yl)-ethyl]-2-isopropoxy-5-(2-methoxy-phenylethynyl)-benzamide
28 N-[1-Hydroxymethyl-2-(1H-pyrrolo[3,2-c]pyridin-3-yl)-ethyl]-2-isopropoxy-5-phenylethynyl-benzamide;
29 2-(3-Fluoro-4-methoxy-phenyl)-6-methoxy-quinazoline-4-carboxylic acid [2-hydroxy-1-(1H-pyrrolo[3,2-c]pyridin-3-ylmethyl)-ethyl]-amide;
30 6-Bromo-2-(3,4,5-trimethoxy-phenyl)-quinoline-4-carboxylic acid [2-hydroxy-1-(1H-pyrrolo[3,2-c]pyridin-3-ylmethyl)-ethyl]-amide;
31 2-(7-Methoxy-benzofuran-2-yl)-6-trifluoromethoxy-quinoline-4-carboxylic acid [1-hydroxymethyl-2-(1H-pyrrolo[3,2-c]pyridin-3-yl)-ethyl]-amide;
32 5′-Fluoro-4-isopropoxy-biphenyl-3,3′-dicarboxylic acid 3-{[2-hydroxy-1-(1H-pyrrolo[3,2-c]pyridin-3-ylmethyl)-ethyl]-amide}3′-methylamide;
33 3′-Chloro-4-isopropoxy-biphenyl-3,4′-dicarboxylic acid 3-{[2-hydroxy-1-(1H-pyrrolo[3,2-c]pyridin-3-ylmethyl)-ethyl]-amide}4′-methylamide;
34 7-(3,4-Dimethoxy-phenyl)-2,3,4,5-tetrahydro-benzo[b]oxepine-9-carboxylic acid [1-hydroxymethyl-2-(1H-pyrrolo[3,2-c]pyridin-3-yl)-ethyl]-amide;
35 4,3′-Dichloro-biphenyl-3,4′-dicarboxylic acid 4′-amide 3-{[2-hydroxy-1-(1H-pyrrolo[3,2-c]pyridin-3-ylmethyl)-ethyl]-amide};
36 N-[2-Hydroxy-1-(1H-pyrrolo[2,3-b]pyridin-3-ylmethyl)-ethyl]-5-(4-methylcarbamoyl-phenylethynyl)-2-trifluoromethoxy-benzamide;
37 4-Isopropoxy-3′,4′-dimethoxy-biphenyl-3-carboxylic acid [2-hydroxy-1-(1H-pyrrolo[2,3-c]pyridin-3-ylmethyl)-ethyl]-amide;
38 4,3′-Dichloro-biphenyl-3,4′-dicarboxylic acid 4′-amide 3-{[2-hydroxy-1-(1H-pyrrolo[2,3-c]pyridin-3-ylmethyl)-ethyl]-amide};
39 3′-Chloro-4-isopropoxy-biphenyl-3,4′-dicarboxylic acid 3-{[2-hydroxy-1-(1H-pyrrolo[2,3-c]pyridin-3-ylmethyl)-ethyl]-amide}4′-methylamide;
40 5′-Fluoro-4-isopropoxy-biphenyl-3,3′-dicarboxylic acid 3-{[2-hydroxy-1-(1H-pyrrolo[2,3-c]pyridin-3-ylmethyl)-ethyl]-amide}3′-methylamide;
41 6-Bromo-2-(3,4,5-trimethoxy-phenyl)-quinoline-4-carboxylic acid [2-hydroxy-1-(1H-pyrrolo[2,3-c]pyridin-3-ylmethyl)-ethyl]-amide;
42 N-[2-Hydroxy-1-(1H-pyrrolo[2,3-c]pyridin-3-ylmethyl)-ethyl]-2-isopropoxy-5-(2-methoxy-phenylethynyl)-benzamide.

### Pharmacological Investigations

HTRF Assay for Measuring cAMP in Cells

The method is based on a competitive immunoassay between native cAMP, which has been produced by the cells, and cAMP which is labelled with XL665. The tracer binding was visualized by a monoclonal antibody, anti-cAMP labelled with cryptate [HTRF=homogeneous time-resolved fluorescence].

The specific signal is inversely proportional to the cAMP concentration of the samples employed. The 665 nm/620 nm fluorescence ratio was evaluated.

The following material was used: 96-well plates for the tissue culture, 96-well plates with black edge and black base (e.g. Fluotrac 600 from Greiner), 96-well plates for the substance dilutions of polypropylene and cAMP Femtomolar (4000 wells Kit, CIS Bio International #62AM1PEC).

The following reagents were used: BSA (bovine serum albumin) Fraction V protease-free, IBMX (3-isobutyl-1-methylxanthine), hFSH (human follicle stimulating hormone), Triton X-100 analytical grade, potassium fluoride analytical grade, G 418 (Geneticin) and Accutase.

Buffer 1 (washing and testing buffer) contained PBS, 1 mM CaCl₂, 1 mM MgCl₂, 0.2% glucose; 0.1% BSA, 1 mM IBMX.
Buffer 2 (2× lysis buffer) contained 1% Triton X-100 in PBS (without CaCl₂ and MgCl₂).
Buffer 3 (assay buffer) contained 50 mM potassium phosphate buffer (pH 7.0); 800 mM potassium fluoride; 0.2% BSA (always added fresh).

### Procedure:

On day 1, the cells were seeded in 96-well plates (3×10⁴ cells per well hFSHR clone 16 cells (CHO cells stably transfected with the human FSH receptor in 150 μl of medium).

The next day, test substance dilutions were made up. For this purpose, all the substances were diluted in ice-cold buffer 1 (with or without hFSH), and the substance dilutions were placed on ice until applied to the cells.

The cell supernatant was then aspirated off, and the cells were washed 2× with 200 μl of buffer 1. The cells were treated with 60 μl of the appropriate substance concentrations at 37° C. for 2 h. The cells were then lysed with 60 μl of buffer 2 (put onto the supernatant) (on a plate shaker at RT for 30 min).

The test conjugates (XL-665 and anti-cAMP cryptate) were diluted in buffer 3 in accordance with the manufacturers' information. The actual mixture for measurement was pipetted into a black 96-well plate (in each case 15 μl of the cell lysate diluted with 35 μl of buffer 1; firstly 25 μl of XL-665 conjugate were pipetted and, after 10 min, 25 μl of the anti-cAMP cryptate were added). This is followed by incubation at RT for 90 minutes. The measurement was carried out in a PheraStar (BMG).

### Tissue Culture Conditions

| | |
|---|---|
| 1) hFSHr clone 16 | Ham's F12 |
| | PSG |
| | 10% FCS |
| | 700 μg/ml G 418 (Geneticin) from PAA. |

Dose-effect curve (hFSH) for the human receptor: 1e-8, 3e-9, 1e-9, 3e-10, 1e-10, 3e-11, 1e-11, 3e-12 mol/l.

The test substances were employed in suitable dilutions in the absence (test for agonism) and in the presence of 1e-9 mol/l hFSH.

### Evaluation

The values of the well ratio were averaged and then entered directly in SigmaPlot versus the concentrations. The maximum and minimum values were determined for each plate, and half the difference is to be regarded as IC₅₀.

The test results (Table 1) show that the compounds according to the invention have an FSH-antagonistic effect.

TABLE 1
FSH-antagonistic effect of selected compounds in the HTRF assay

| Compound [Ex. #] | IC₅₀ | |
|---|---|---|
| 1 | 1.5 | μM |
| 3 | 4 | μM |
| 5 | 65 | nM |
| 6 | 6 | μM |
| 9 | 8 | μM |
| 12 | 10 | μM |
| 14 | 5 | μM |
| 16 | 9 | μM |
| 21 | 5 | μM |
| 22 | 200 | nM |
| 24 | 70 | nM |
| 26 | 1 | μM |
| 39 | 6.5 | μM |
| 42 | 5 | μM |

Being antagonists of the FSH receptor, compounds of the general formula I or pharmaceutically acceptable salts thereof can thus be used for the manufacture of medicaments to be used for the fertility control in male and/or in a female animals, in particular in men and/or women; as well as for the treatment and/or prevention of osteoporosis.

### Dosage

Satisfactory results are generally to be expected if the daily doses comprise a range from 5 μg to 50 mg of the compound according to the invention per kg of body weight. A recommended daily dose for larger mammals, for example humans, is in the range from 10 μg to 30 mg per kg of body weight. Suitable dosages for the compounds according to the invention are from 0.005 to 50 mg per day per kg of body weight, depending on the age and constitution of the patient, it being possible to administer the necessary daily dose by single or multiple delivery.

Pharmaceutical products based on the novel compounds are formulated in a manner known per se by processing the active ingredient with the carrier substances, fillers, substances which influence disintegration, binders, humectants, lubricants, absorbents, diluents, test modifiers, colorants etc. which are used in pharmaceutical technology, and converting into the desired administration form. Reference should be made in this connection to Remington's Pharmaceutical Science, 15^{th} ed. Mack Publishing Company, East Pennsylvania (1980).

Suitable for oral administration are in particular tablets, coated tablets, capsules, pills, powders, granules, pastilles, suspensions, emulsions or solutions. Preparations for injection and infusion are possible for parenteral administration. Appropriately prepared crystal suspensions can be used for intraarticular injection. Aqueous and oily solutions for injection or suspensions and corresponding depot preparations can be used for intramuscular injection. The novel compounds can be used for rectal administration in the form of suppositories, capsules, solutions (e.g. in the form of enemas) and ointments both for systemic and for local therapy. Formulations possible for topical application are gels, ointments, greasy ointments, creams, pastes, dusting powders, milk and tinctures. The dosage of the compounds of the general formula I in these preparations should be 0.01%-20% in order to achieve an adequate pharmacological effect. Topical use can also take place by means of a transdermal system, for example a patch.

The invention likewise encompasses the compounds according to the invention of the general formula I as therapeutic active ingredient. The invention further includes the compounds according to the invention of the general formula I as therapeutic active ingredients together with pharmaceutically suitable and acceptable excipients and carriers. The invention likewise encompasses a pharmaceutical composition which comprises one of the pharmaceutically active compounds according to the invention or mixture thereof and a pharmaceutically suitable salt or pharmaceutically suitable excipients and carriers.

The present invention therefore also relates to pharmaceutical compositions which comprise at least one compound of the general formula I, where appropriate together with pharmaceutically suitable excipients and/or carriers.

Suitable for forming pharmaceutically suitable salts of the compounds according to the invention of the general formula I are, by methods known to the skilled person, as inorganic acids inter alia hydrochloric acid, hydrobromic acid, sulphuric acid and phosphoric acid, nitric acid, as carboxylic acids inter alia acetic acid, propionic acid, hexanoic acid, octanoic acid, decanoic acid, oleic acid, stearic acid, maleic acid, fumaric acid, succinic acid, benzoic acid, ascorbic acid, oxalic acid, salicylic acid, tartaric acid, citric acid, lactic acid, glycolic acid, malic acid, mandelic acid, cinnamic acid, glutamic acid, aspartic acid, and as sulphonic acids inter alia methanesulphonic acid, ethanesulphonic acid, toluenesulphonic acid, benzenesulphonic acid and naphthalenesulphonic acid.

These pharmaceutical compositions and medicaments may be intended for oral, rectal, subcutaneous, transdermal, percutaneous, intravenous or intramuscular administration. They comprise besides conventional carriers and/or diluents at least one compound of the general formula I.

The medicaments of the invention are produced using the customary solid or liquid carriers or diluents and the excipients customarily used in pharmaceutical technology, in accordance with the desired mode of administration with a suitable dosage in a known manner. The preferred preparations consist of a dosage form which is suitable for oral administration. Examples of such dosage forms are tablets, film-coated tablets, sugarcoated tablets, capsules, pills, powders, solutions or suspensions or else depot forms.

The pharmaceutical compositions which comprise at least one of the compounds according to the invention are preferably administered orally.

Parenteral preparations such as solutions for injection are also suitable. Preparations which may also be mentioned for example are suppositories.

Appropriate tablets can be obtained for example by mixing the active ingredient with known excipients, for example inert diluents such as dextrose, sugar, sorbitol, mannitol, polyvinylpyrrolidone, disintegrants such as maize starch or alginic acid, binders such as starch or gelatine, lubricants such as magnesium stearate or talc and/or agents to achieve a depot effect such as carboxylpolymethylene, carboxylmethylcellulose, cellulose acetate phthalate or polyvinyl acetate. The tablets may also consist of a plurality of layers.

Correspondingly, coated tablets can be produced by coating cores which have been produced in analogy to the tablets with agents normally used in tablet coatings, for example polyvinylpyrrolidone or shellac, gum Arabic, talc, titanium oxide or sugar. The tablet coating may also consist of a plurality of layers, it being possible to use the excipients mentioned above for tablets.

Solutions or suspensions with the compounds according to the invention of the general formula I may additionally comprise taste-improving agents such as saccharin, cyclamate or sugar and, for example, flavourings such as vanillin or orange extract. They may additionally comprise suspending aids such as sodium carboxymethylcellulose or preservatives such as p-hydroxybenzoates.

Capsules comprising the compounds of the general formula I can be produced for example by the compound(s) of the general formula I being mixed with an inert carrier such as lactose or sorbitol and encapsulated in gelatine capsules.

Suitable suppositories can be produced for example by mixing with carriers intended for this purpose, such as neutral fats or polyethylene glycol or derivatives thereof.

The present invention also relates to processes for preparing the compounds according to the invention.

Compounds of the general formula I can be prepared as shown in Scheme 1 by an amide-formation reaction between the amino alcohol VI and the carboxylic acid VII.

Reagents suitable for this purpose are all suitable peptide-coupling reagents which are known to the skilled person and which convert the carboxylic acid, where appropriate in the presence of a base, into an intermediate active ester, for example PyBOP ([(1H-benzotriazol-1-yl)oxy]tris(pyrrolidin-1-yl)phosphonium hexafluorophosphate), HATU (2-(7-aza-1H-benzotriazol-1-yl)-1 1,3,3-tetramethyluronium hexafluorophosphate), HBTU (2-(1H-benzotriazol-1-yl)-1,1,3,3-tetramethyluronium hexafluorophosphate), EDC (N-[3-(dimethylamino)propyl]-N′-ethylcarbodiimide hydrochloride)/HOBt (1-hydroxy-1H-benzotriazole). It is possible as alternative for the carboxylic acid VII to be converted, where appropriate in the presence of a base, into the carbonyl chloride and reacted with the amino alcohol VI to give the product of the general formula I.

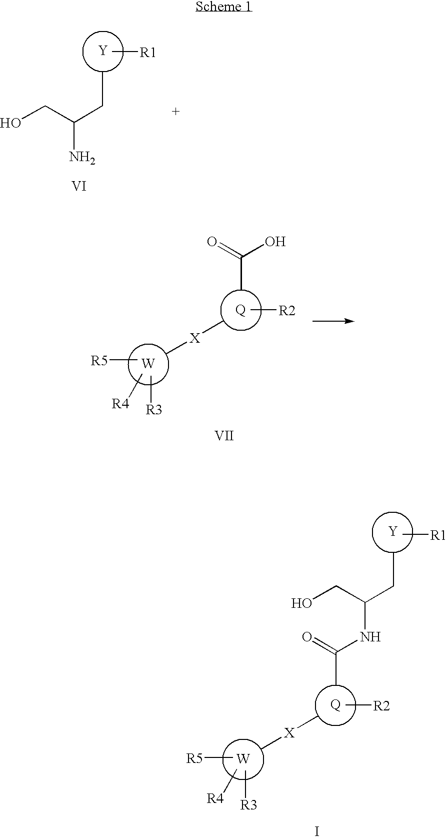

Compounds of general formula II can be prepared as shown in Scheme 2 by an amide-formation reaction between the amino alcohol VI and carboxylic acid VIII. Reagents suitable for this purpose are all known peptide-coupling reagents which convert the carboxylic acid, where appropriate in the presence of a base, into an intermediate active ester, for example PyBOP ([(1H-benzotriazol-1-yl)oxy]tris(pyrrolidin-1-yl)phosphonium hexafluorophosphate), HATU (2-(7-aza-1H-benzotriazol-1-yl)-1,1,3,3-tetramethyluronium hexafluorophosphate), HBTU (2-(1H-benzotriazol-1-yl)-1,1,3,3-tetramethyluronium hexafluorophosphate), EDC (N-[3-(dimethylamino)propyl]-N′-ethylcarbodiimide hydrochloride)/HOBt (1-hydroxy-1H-benzotriazole). It is possible as alternative for the carboxylic acid VIII to be converted, where appropriate in the presence of a base, into the carbonyl chloride and reacted with the amino alcohol VI to give the product of the general formula II.

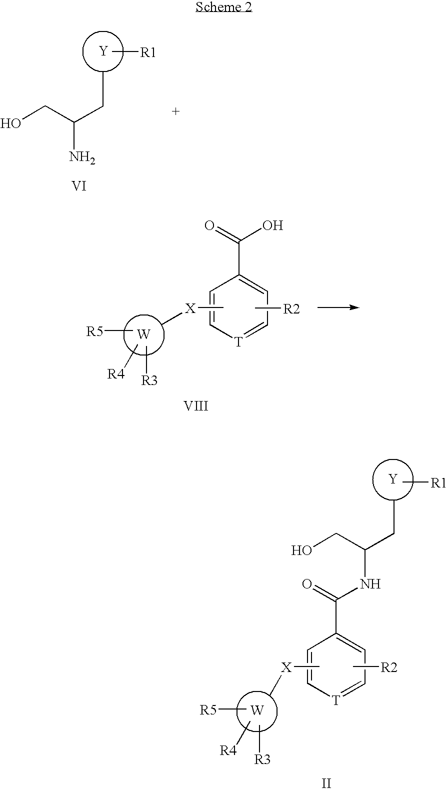

Compounds of general formula III can be prepared as shown in Scheme 3 by an amide-formation reaction between the amino alcohol VI and carboxylic acid IX. Reagents suitable for this purpose are all suitable peptide-coupling reagents which are known to the skilled person and which convert the carboxylic acid, where appropriate in the presence of a base, into an intermediate active ester, for example PyBOP ([(1H-benzotriazol-1-yl)oxy]tris(pyrrolidin-1-yl)phosphonium hexafluorophosphate), HATU (2-(7-aza-1H-benzotriazol-1-yl)-1,1,3,3-tetramethyluronium hexafluorophosphate), HBTU (2-(1H-benzotriazol-1-yl)-1,1,3,3-tetramethyluronium hexafluorophosphate), EDC (N-[3-(dimethylamino)propyl]-N′-ethylcarbodiimide hydrochloride)/HOBt (1-hydroxy-1H-benzotriazole). It is possible as alternative for the carboxylic acid IX to be converted, where appropriate in the presence of a base, into the carbonyl chloride and reacted with the amino alcohol VI to give the product of the general formula III.

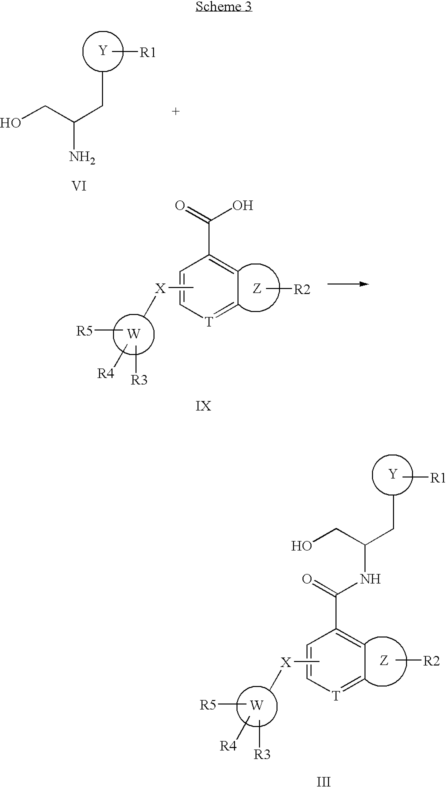

Compounds of general formula IV can be prepared as shown in Scheme 4 by an amide-formation reaction between the amino alcohol VI and carboxylic acid X. Reagents suitable for this purpose are all suitable peptide-coupling reagents which are known to the skilled person and which convert the carboxylic acid, where appropriate in the presence of a base, into an intermediate active ester, for example PyBOP ([(1H-benzotriazol-1-yl)oxy]tris(pyrrolidin-1-yl)phosphonium hexafluorophosphate), HATU (2-(7-aza-1H-benzotriazol-1-yl)-1,1,3,3-tetramethyluronium hexafluorophosphate), HBTU (2-(1H-benzotriazol-1-yl)-1,1,3,3-tetramethyluronium hexafluorophosphate), EDC (N-[3-(dimethylamino)propyl]-N′-ethylcarbodiimide hydrochloride)/HOBt (1-hydroxy-1H-benzotriazole). It is possible as alternative for the carboxylic acid X to be converted, where appropriate in the presence of a base, into the carbonyl chloride and reacted with the amino alcohol VI to give the product of the general formula IV.

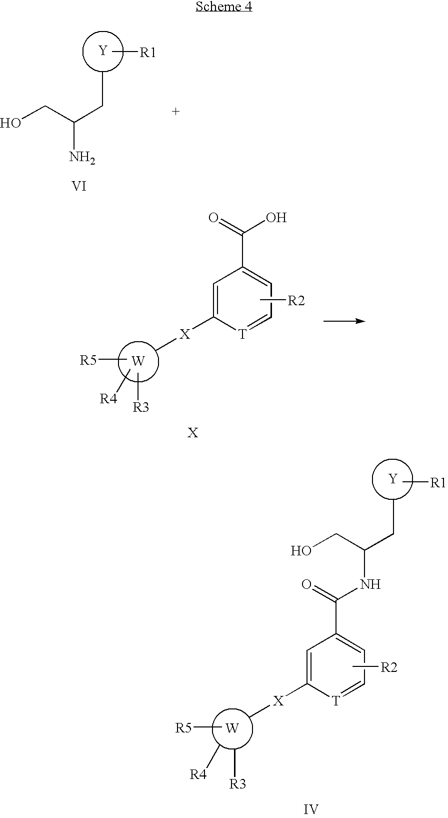

Compounds of general formula V can be prepared as shown in Scheme 5 by an amide-formation reaction between the amino alcohol VI and carboxylic acid XI. Reagents suitable for this purpose are all suitable peptide-coupling reagents which are known to the skilled person and which convert the carboxylic acid, where appropriate in the presence of a base, into an intermediate active ester, for example PyBOP ([(1H-benzotriazol-1-yl)oxy]tris(pyrrolidin-1-yl)phosphonium hexafluorophosphate), HATU (2-(7-aza-1H-benzotriazol-1-yl)-1,1,3,3-tetramethyluronium hexafluorophosphate), HBTU (2-(1H-benzotriazol-1-yl)-1,1,3,3-tetramethyluronium hexafluorophosphate), EDC (N-[3-(dimethylamino)propyl]-N′-ethylcarbodiimide hydrochloride)/HOBt (1-hydroxy-1H-benzotriazole). It is possible as alternative for the carboxylic acid XI to be converted, where appropriate in the presence of a base, into the carbonyl chloride and reacted with the amino alcohol VI to give the product of the general formula V.

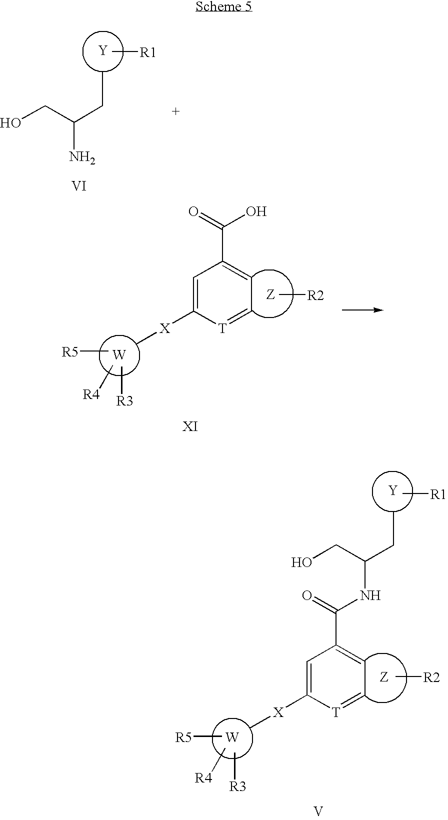

Compounds of the general formula I can in principle be prepared as shown in Scheme 6 by an amide-formation reaction between amino alcohol VI and a carboxylic acid VII. The reagents typically used for the coupling are EDC and HOBt.

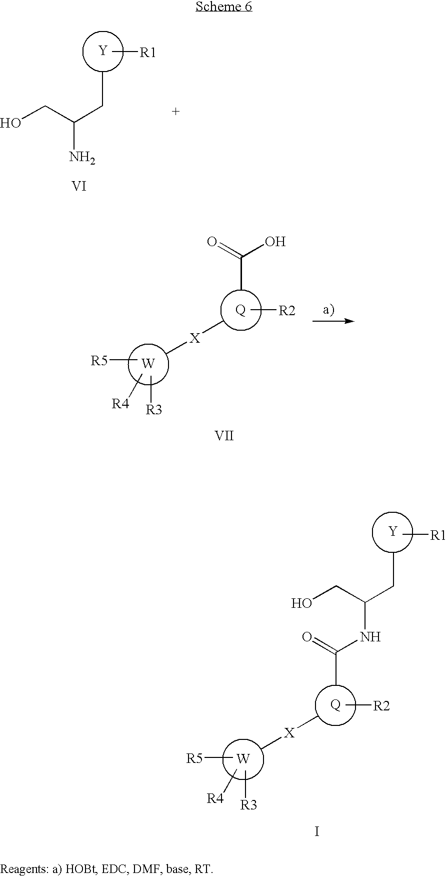

Compounds of the general formula I can in principle also be prepared as shown in Scheme 7 by an amide-formation reaction between amino ester XII and a carboxylic acid VII. The resulting carboxylic ester XIII is subsequently reduced to the corresponding alcohol I with suitable reducing agents such as lithium borohydride.

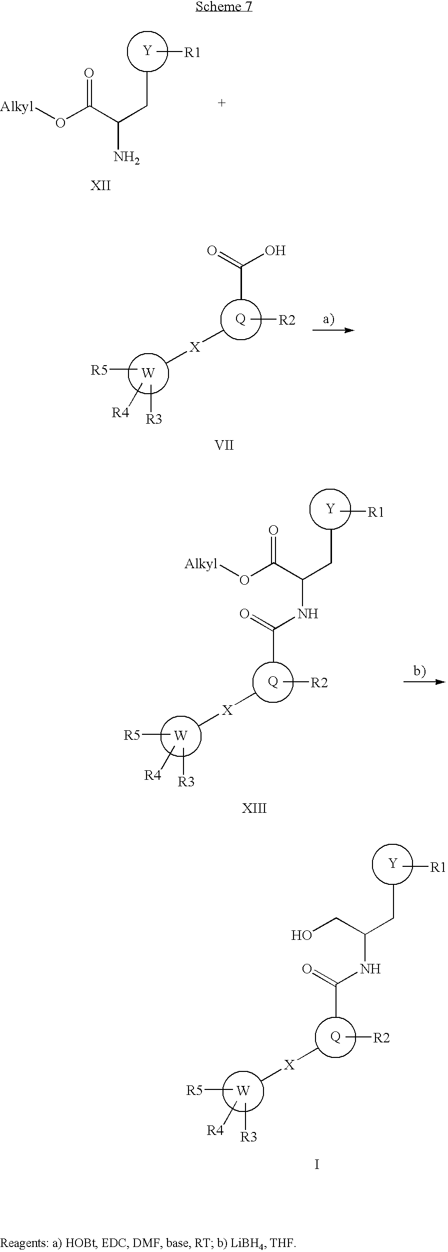

Amino alcohols of the general formula VI can in principle be prepared as shown in Scheme 8 from amino acids which are commercially available or described in the literature.

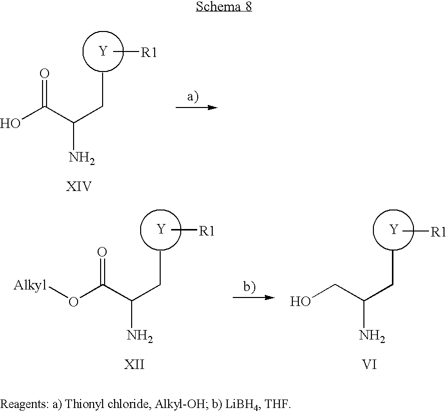

The carboxylic acids of the general formula VII can be prepared as shown in Scheme 9 by a Suzuki reaction between a boronic acid XV or XVIII and corresponding halogen compound XVI or XVII (Hal=I, Br, Cl) to form ester XIX which can be converted to carboxylic acid VII via an ester hydrolysis reaction with KOH in methanol.

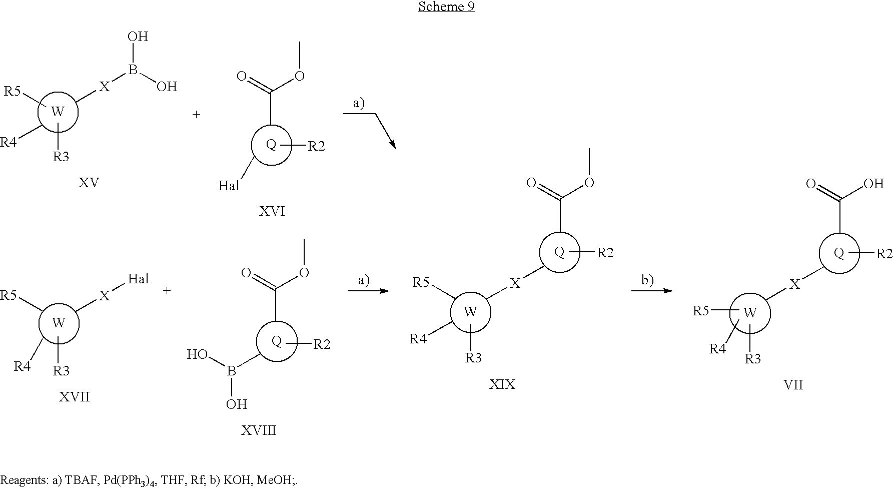

The carboxylic acid derivatives of formula XXIV can in principle be prepared according to Scheme 10 via a Sonogashira type coupling of acetylenes XX or XXII with their corresponding aryl halides XVI or XXI with subsequent hydrolysis of the resulting carboxylic esters XXIII.

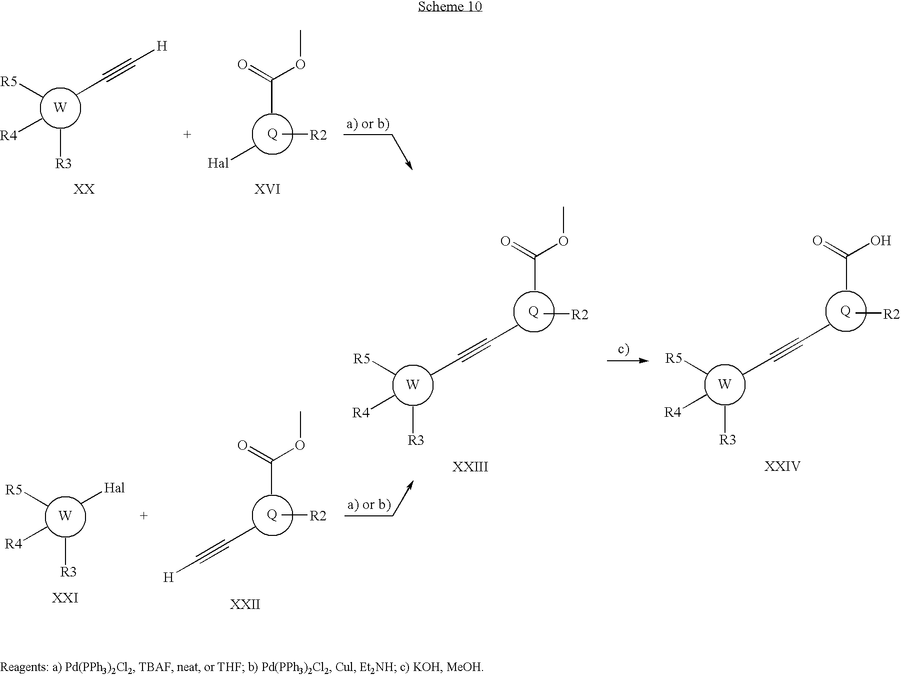

Carboxylic acids of the formula XXVII can be prepared as shown in Scheme 11 in a so-called Pfitzinger reaction from a methyl ketone XXV and an isatin derivative XXVI.

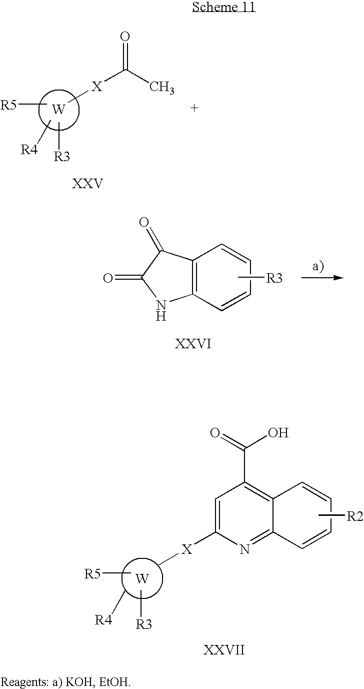

Amino alcohols of formula I can in principle also be prepared according to scheme 12 starting from protected amino acids XXVIII which are commercially available or described in the literature. Amino acid XXVIII is coupled to solid phase resin such as Wang resin via a peptide coupling (see XXIX) and subsequently deprotected using piperidine (see XXX). The free amine XXX is then reacted with a corresponding carboxylic acid VII in a peptide coupling reaction to provide amides XXXI. After reductive cleavage with DIBAH amino alcohols of formula I were obtained.

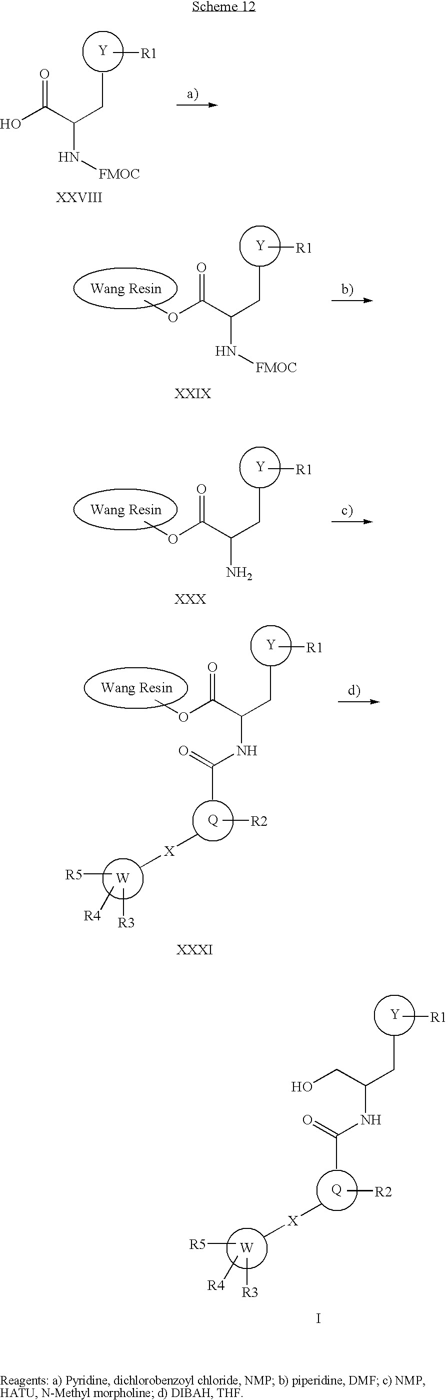

Azatryptophanols of formula XXXVI can in principle be prepared according to scheme 13 starting from commercially available azaindoles XXXII. The terms azatryptophanol and azaindol XXXII include all possible isomers regarding the position of the nitrogen atom in the six-membered ring (depicted by N in the centre of the six-membered ring in formulae in scheme 13). After formylation and reductive amination amine XXXIII is obtained which is transformed to carboxylic ester XXXIV via a substitution reaction. After ester and amide hydrolysis and decarboxylation amino ester XXXV is obtained which is converted into amino alcohol XXXVI by an ester reduction with lithium borohydride.

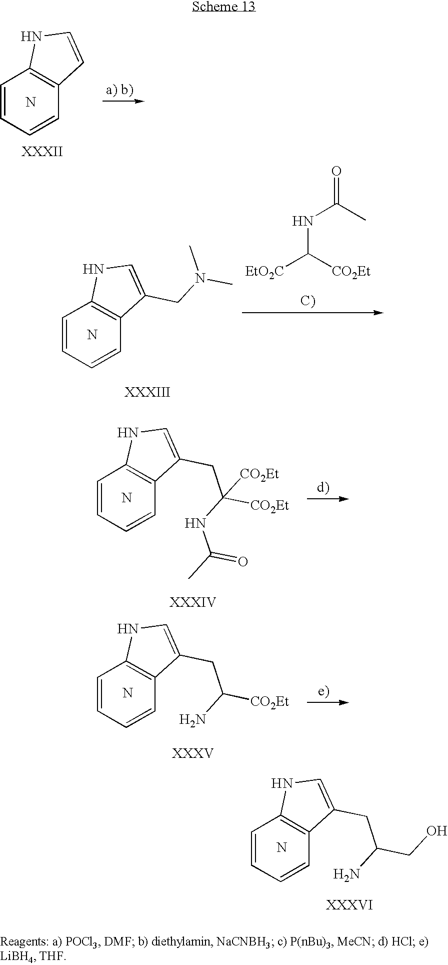

The compounds according to the invention of the general formula I can be prepared as described below.

### Abbreviations Used

ACN Acetonitrile
DIBAC Diisobutylaluminium hydride
DMF N,N-Dimethylformamide
EDC N-Ethyl-N′-(3-dimethylaminopropyl)carbodiimide
EtOH Ethanol
HATU O-(7-Azabenzotriazol-1-yl)-N,N,N′,N′-tetramethyluronium hexafluorophosphate
FMOC (9H-Fluoren-9-ylmethoxy)carbonyl
HOBt 1-Hydroxy-1H-benzotriazole
MeCN Acetonitrile
MeOH Methanol
MTBE Methyl tert-butyl ether
NMM 4-methylmorpholine
NMP N-Methylpyrrolidinone
pTsOH para-toluene sulfonic acid
Rf Reflux
RT Room temperature
TBAF Tetrabutylammonium fluoride
TFA Trifluoroacetic acid
THF Tetrahydrofuran

### Synthesis of the Compounds According to the Invention

### EXAMPLE 1

### 5′-Fluoro-4-isopropoxy-biphenyl-3,3′-dicarboxylic acid 3-{[2-hydroxy-1-(1H-pyrrolo[2,3-b]pyridin-3-ylmethyl)-ethyl]-amide}3′-methylamide

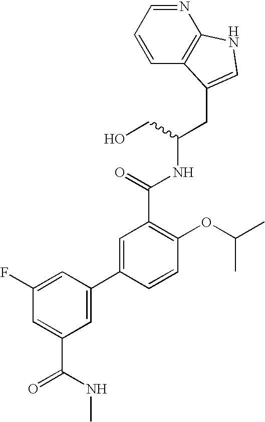

### 1a) 5-Iodo-2-isopropoxy-benzoic acid methyl ester

A suspension of 5-Iodo-2-hydroxy benzoic acid methyl ester (50 g), potassium carbonate (74.6 g) and iso-propyl iodide (89.9 ml) in acetone (500 ml) was stirred under reflux overnight. The reaction mixture was allowed to cool down to ambient temperature and the solid was removed by filtration. The filtrate was evaporated and the title compound was obtained in 96% yield (55.1 g). ¹H-NMR (CDCl₃): 8.02 d (J=2.3 Hz, 1H); 7.67 dd (J=2.5 Hz/8.9 Hz, 1H); 6.74 d (J=8.9 Hz, 1H); 4.54 m (1H); 3.87 s (3H); 1.36 m (6H).

### 1b) 3′-Fluoro-4-isopropoxy-5′-methylcarbamoyl-biphenyl-3-carboxylic acid methyl ester

A solution of 5-Iodo-2-isopropoxy-benzoic acid methyl ester (13.6 g), 3-Fluoro-5-methylcarbamoyl benzene boronic acid (9 g) and Pd(PPh₃)₄ (960 mg) in ethanol (150 mL), toluene (200 mL) and an aqueous sodium carbonate solution (2M, 45 mL) were stirred at reflux for 4 h. The solvent was evaporated and the title compound was obtained as crude product in 17 g yield. The compound was used without further purification in the next step.

### 1c) 3′-Fluoro-4-isopropoxy-5′-methylcarbamoyl-biphenyl-3-carboxylic acid

A solution of 3′-Fluoro-4-isopropoxy-5′-methylcarbamoyl-biphenyl-3-carboxylic acid methyl ester (2 g), KOH (4 g) in methanol (30 mL) was stirred at ambient temperature overnight. The solvent was distilled off under reduced pressure and the residue was extracted with water/ether. The water phase was acidified by addition of HCl (4N) and the precipitate filtered off. The title compound was obtained in 70% yield. ¹H-NMR (CDCl₃): 8.40 d (J=2.5 Hz, 1H); 7.77 dd (J=2.5 Hz/8.6 Hz, 1H); 7.73 m (1H); 7.47 m (1H); 7.38 m (1H); 7.14 d (J=8.6 Hz, 1H); 6.49 s (1H); 4.93 m (1H); 3.04 d (J=4.8 Hz, 3H); 1.52 d (J=6.1 Hz, 6H).

### 1d) 2-Amino-3-(1H-pyrrolo[2,3-b]pyridin-3-yl)-propionic acid methyl ester hydrochloride

Commercially available 2-Amino-3-(1H-pyrrolo[2,3-b]pyridin-3-yl)-propionic acid (1.5 g) were dissolved in MeOH (75 mL). SOCl₂ (2.67 mL) was added drop by drop to the suspension at 0° C. The reaction was allowed to warm to ambient temperature and then stirred under reflux for 4 hours. After removal of the solvent under reduced pressure the title compound was obtained in 1.98 g yield. ¹H-NMR (DMSO-d₆): 12.60 s (1H); 8.75 s (2H); 8.51 d (J=7.9 Hz, 1H); 8.42 d (J=5.3 Hz, 1H); 4.33 m (1H); 3.70 s (3H); 3.41 m (2H).

### 1e) 2-Amino-3-(1H-pyrrolo[2,3-b]pyridin-3-yl)-propan-1-ol

2-Amino-3-(1H-pyrrolo[2,3-b]pyridin-3-yl)-propionic acid methyl ester hydrochloride (1.87 g) was suspended in THF (93 mL) and cooled to 0° C. LiAlH₄ (916 mg) were added to the reaction mixture in small portions. The ice bath was removed and the reaction was stirred under reflux for 1 hour. The reaction was cooled to ambient temperature and then carefully quenched with water (0.92 mL) and NaOH (6M, 0.92 mL). The formed precipitate was filtered off and washed with THF to obtain the title compound in 43% yield. ESI-MS [M+1]=192.

### 1f) 5′-Fluoro-4-isopropoxy-biphenyl-3,3′-dicarboxylic acid 3-{[2-hydroxy-1-(1H-pyrrolo[2,3-b]pyridin-3-ylmethyl)-ethyl]-amide}3′-methylamide

A solution of 2-Amino-3-(1H-pyrrolo[2,3-b]pyridin-3-yl)-propan-1-ol (76 mg), 3′-Fluoro-4-isopropoxy-5′-methylcarbamoyl-biphenyl-3-carboxylic acid (70 mg), HOBt (34 mg) and ECDI (48 mg) in DMF (3 mL) was stirred at ambient temperature overnight. Water and ethyl acetate were added to the reaction mixture and the phases separated. The water phase was extracted with ethyl acetate (3×) and the combined organic layers were washed with brine (3×). The organic layer was dried over magnesium sulphate and the solvent was removed. After flash chromatography the title compound was obtained in 27% yield. ¹H-NMR (DMSO-d₆): 11.33 s (1H); 8.67 d (J=4.6 Hz, 1H); 8.41 d (J=8.1 Hz, 1H); 8.23 d (J=2.5 Hz, 1H); 8.13 dd (J=1.5 Hz/4.6 Hz, 1H); 8.07 dd (J=1.3 Hz/7.8 Hz, 1H); 7.93 s (1H); 7.84 dd (J=2.5 Hz/8.6 Hz, 1H); 7.65 m (1H); 7.54 m (1H); 7.27 d (J=9.1 Hz, 1H); 7.23 d (J=2.3 Hz, 1H); 7.00 m (2H); 4.97 m (1H); 4.80 m (1H); 4.22 m (1H); 3.47 m (2H); 2.96 m (2H); 2.78 d (J=4.6 Hz, 3H); 1.22 m (6H).

The following compounds were obtained in analogy to the preparation methods described above:

| | | Method | | |
|---|---|---|---|---|
| | Product; | analogous | | |
| Ex. | reagents | to | ¹H-NMR [ppm] | Structure |
| 2 | 3′-Chloro-4-isopropoxy-biphenyl-3,4′-dicarboxylicacid 3-{[2-hydroxy-1-(1H-pyrrolo[2,3-b]pyridin-3-ylmethyl)-ethyl]-amide}4′-methylamide;2-Amino-3-(1H-pyrrolo[2,3-b]pyridin-3-yl)-propan-1-oland3′-Chloro-4-isopropoxy-4′-methylcarbamoyl-biphenyl-3-carboxylic acid | 1 | ¹H-NMR (DMSO-d₆):11.35 s (1H); 8.41 d (J =8.3 Hz, 1H); 8.35 d (J =4.6 Hz, 1H); 8.14 m(2H); 8.05 d (J = 8.3 Hz,1H); 7.79 dd (J = 8.3 Hz,/8.8 Hz, 1H); 7.70 d (J =1.5 Hz, 1H); 7.61 m(1H); 7.45 d (J = 8.1 Hz,1H); 7.23 m (2H); 6.99m (1H); 4.97 m (1H):4.79 m (1H); 4.21 m(1H); 3.45 m (2H); 2.94m (2H); 2.73 d (J = 4.6Hz, 3H); 1.21 m (6H). | 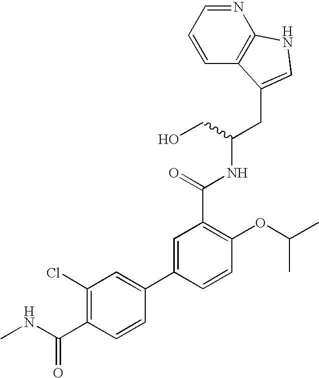 |

### EXAMPLE 3

### N-[1-Hydroxymethyl-2-(1H-pyrrolo[2,3-b]pyridin-3-yl)-ethyl]-2-isopropoxy-5-(2-methoxy-phenylethynyl)-benzamide

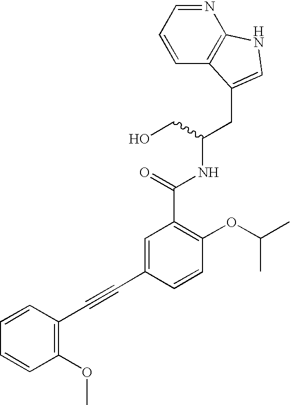

### 3a) 5-Iodo-2-isopropoxy-benzoic acid methyl ester

The title compound was prepared according to the procedure described in example 1a.

### 3b) 2-Isopropoxy-5-(2-methoxy-phenylethynyl)-benzoic acid methyl ester

5-Iodo-2-isopropoxy-benzoic acid methyl ester (10.09 g), 2-methoxyphenyl acetylene (5 g), palladium dichlorobis(triphenylphosphine) (664 mg) and TBAF×3 water (24.7 g) were stirred under reflux for 5 hours. The reaction mixture was concentrated and extracted with ethylacetate/water. The combined organic layers were dried over sodium sulphate and the solvent was evaporated. The title compound was obtained in 35% yield after flash chromatography. ¹H-NMR (CDCl₃): 7.97 d (J=2.3 Hz, 1H); 7.58 dd (J=2.3 Hz/10.9 Hz, 1H); 7.49 dd (J=1.7 Hz/7.5 Hz, 1H); 7.30 m (1H); 6.93 m (3H); 4.62 m (1H); 3.91 s (3H); 3.88 s (3H); 1.39 m (6H).

### 3c) 2-Isopropoxy-5-(2-methoxy-phenylethynyl)-benzoic acid

A solution of 2-Isopropoxy-5-(2-methoxy-phenylethynyl)-benzoic acid methyl ester (860 mg) and KOH (1.49 g) in MeOH (25 mL) was stirred under reflux overnight. The solvent was distilled off and the remaining solid was dissolved in water and extracted with ethylacetate. The water phase was acidified by addition of HCl (2 molar) and the water phase was extracted with ethylacetate (3×50 ml). The combined organic layers were dried over sodium sulphate and evaporated to dryness. The title compound was obtained in 73% yield. ¹H-NMR (DMSO-d₆): 7.69 d (J=2.3 Hz, 1H); 7.60 dd (J=2.3 Hz/8.7 Hz, 1H); 7.48 dd (J=1.7 Hz/7.5 Hz, 1H); 7.38 m (1H); 7.18 d (J=8.9 Hz, 1H); 7.09 d (J=8.1 Hz, 1H); 6.98 m (1H); 4.72 m (1H); 3.86 s (3H); 1.29 m (6H).

### 3d) N-[1-Hydroxymethyl-2-(1H-pyrrolo[2,3-b]pyridin-3-yl)-ethyl]-2-isopropoxy-5-(2-methoxy-phenylethynyl)-benzamide

A solution of 5-Isopropoxy-5-(2-methoxy-phenylethynyl)-benzoic acid (66 mg), 2-Amino-3-(1H-pyrrolo[2,3-b]pyridin-3-yl)-propan-1-ol (45 mg), HOBt (35 mg) and EDCI (49 mg) in DMF (2.7 ml) was stirred overnight at ambient temperature. The reaction mixture was poured into water, extracted with ethyl acetate (3×). The combined organic layers were washed with brine and the solvent removed under vacuum. The title compound was obtained in 47% yield after flash chromatography. ¹H-NMR (DMSO-d₆): 11.34 s (1H); 8.34 d (J=8.1 Hz, 1H); 8.13 d (J=4.6 Hz, 1H); 8.02 d (J=7.8 Hz, 1H); 7.92 d (J=2.0 Hz, 1H); 7.54 dd (J=2.3 Hz/8.3 Hz, 1H); 7.44 d (J=7.6 Hz, 1H); 7.34 m (1H); 7.22 m (1H); 7.16 d (J=8.8 Hz, 1H); 7.04 d (J=8.3 Hz, 1H); 6.99 m (1H); 6.93 m (1H); 4.97 m (1H); 4.77 m (1H); 4.20 m (1H); 3.82 s (3H); 3.44 m (2H); 2.92 m (2H); 1.19 m (6H).

The following compounds were obtained in analogy to the preparation methods described above:

| | | Method | | |
|---|---|---|---|---|
| | Product; | analogous | | |
| Ex. | reagents | to | ¹H-NMR [ppm] | Structure |
| 4 | N-[1-Hydroxymethyl-2-(1H-pyrrolo[2,3-b]pyridin-3-yl)-ethyl]-2-isopropoxy-5-phenylethynyl-benzamide;2-Amino-3-(1H-pyrrolo[2,3-b]pyridin-3-yl)-propan-1-oland2-Isopropoxy-5-phenylethynyl-benzoic acid | 3 | ¹H-NMR (DMSO-d₆):11.34 s (1H); 8.33 d (J =8.1 Hz, 1H); 8.14 d(J = 4.6 Hz, 1H); 8.02d (J = 7.6 Hz, 1H);7.96 s (1H); 7.58 d (J =8.6 Hz, 1H); 7.51 m(2H); 7.38 m (3H);7.23 s (1H); 7.18 d (J =8.8 Hz, 1H); 6.98 m(1H); 4.96 m (1H);4.76 m (1H); 4.19 m(1H); 3.44 m (2H);2.96 m (2H); 1.19 m(6H). | 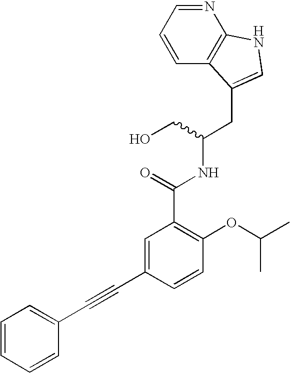 |

### EXAMPLE 5

### 6-(3-Chloro-4-methylcarbamoyl-phenyl)-2,2-dimethyl-2H-chromene-8-carboxylic acid [2-hydroxy-1-(1H-pyrrolo[2,3-b]pyridin-3-ylmethyl)-ethyl]-amide

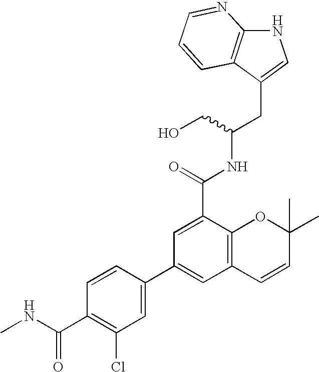

### 5a) 2-(1,1-Dimethyl-prop-2-ynyloxy)-5-iodo-benzoic acid methyl ester

Ethynylcarbinol (10.0 g, 0.0358 mol), methyl-5-iodosalicylate (2.51 g, 0.0359 mol) and Ph₃P (9.43 g, 0.0360 mol) were dissolved in 15 ml of dry toluene under argon atmosphere with stirring. Diisopropyl azodicarboxylate 7.27 g (0.036 mol) was added drop-wise at 10° C. The mixture was stirred overnight under argon atmosphere. The precipitate was filtered off and washed with 15 ml of dry, cooled toluene. The solvent was removed under reduced pressure. The residue was purified by column chromatography over silica gel (hexane/EtOAc 99:1). Yield of the title compound was 46.8%. ESI-MS: 345 [M+1].

### 5b) 6-Iodo-2,2-dimethyl-2H-chromene-8-carboxylic acid methyl ester

A solution of 2-(1,1-Dimethyl-prop-2-ynyloxy)-5-iodo-benzoic acid methyl ester (1.37 g) in diethylamino benzene (18 mL) was stirred in a preheated metal bath at 250° C. for 90 minutes. The reaction mixture was cooled to ambient temperature and poured onto ice/concentrated HCl. The mixture was extracted with ethyl acetate and the solvent was removed under reduced pressure. The title compound was obtained in 86% yield (1.19 g) after flash chromatography. ¹H-NMR (CDCl₃): 7.91 d (J=2.3 Hz, 1H); 7.37 d (J=2.3 Hz, 1H); 6.24 d (J=10.0 Hz, 1H); 5.71 d (J=10.0 Hz, 1H); 3.87 s (3H); 1.46 s (6H).

### 5c) 6-(3-Chloro-4-methylcarbamoyl-phenyl)-2,2-dimethyl-2H-chromene-8-carboxylic acid methyl ester

A solution of 6-Iodo-2,2-dimethyl-2H-chromene-8-carboxylic acid methyl ester (1 g), 3-Chloro-4-methylcarbamoyl benzene boronic acid (682 mg) and Pd(PPh₃)₄ (67 mg) in ethanol (6 mL), toluene (6 mL) and an aqueous sodium carbonate solution (2M, 2.9 mL) were stirred at reflux for 20 minutes. The solvent was evaporated and the title compound was obtained after flash chromatography in 74% yield. ¹H-NMR (CDCl₃): 7.91 d (J=2.5 Hz, 1H); 7.74 d (J=8.1 Hz, 1H); 7.56 d (J=1.7 Hz, 1H); 7.50 dd (J=1.7 Hz/8.1 Hz, 1H); 7.30 d (J=2.5 Hz, 1H); 6.38 d (J=10.0 Hz, 1H); 5.75 d (J=9.8 Hz, 1H); 3.91 s (3H); 3.04 d (J=4.9 Hz, 3H); 1.50 s (6H).

### 5d) 6-(3-Chloro-4-methylcarbamoyl-phenyl)-2,2-dimethyl-2H-chromene-8-carboxylic acid

A solution of 6-(3-Chloro-4-methylcarbamoyl-phenyl)-2,2-dimethyl-2H-chromene-8-carboxylic acid methyl ester (830 mg) and KOH (10% in MeOH, 10 mL) was stirred at ambient temperature overnight. The solvent was distilled off and the remaining solid was dissolved in water and extracted with ethylacetate. The water phase was acidified by addition of HCl (2 molar) and the water phase was extracted with ethylacetate (3×50 ml). The combined organic layers were dried over sodium sulphate and evaporated to dryness. The title compound was obtained in 85% yield. ¹H-NMR (DMSO-d₆): 12.72 s (1H); 8.34 d (J=4.5 Hz, 1H); 7.74 m (2H); 7.63 m (2H); 7.42 d (J=8.1 Hz, 1H); 6.49 d (J=10.0 Hz, 1H); 5.88 d (J=10.0 Hz, 1H); 2.72 d (J=4.7 Hz, 3H); 1.38 s (6H).

### 5e) 6-(3-Chloro-4-methylcarbamoyl-phenyl)-2,2-dimethyl-2H-chromene-8-carboxylic acid [2-hydroxy-1-(1H-pyrrolo[2,3-b]pyridin-3-ylmethyl)-ethyl]-amide

A solution of 6-(3-Chloro-4-methylcarbamoyl-phenyl)-2,2-dimethyl-2H-chromene-8-carboxylic acid (200 mg), 2-Amino-3-(1H-pyrrolo[2,3-b]pyridin-3-yl)-propan-1-ol (123 mg), HOBt×water (98 mg) and EDCI (124 mg) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 29% yield after flash chromatography. ¹H-NMR (DMSO-d₆): 11.33 s (1H); 8.53 d (J=8.1 Hz, 1H); 8.40 d (J=4.5 Hz, 1H); 8.20 dd (J=4.7 Hz/1.5 Hz, 1H); 8.12 dd (J=7.9 Hz/1.3 Hz, 1H); 8.07 d (J=2.5 Hz, 1H); 7.76 d (J=1.7 Hz, 1H); 7.69 d (J=2.5 Hz, 1H); 7.65 dd (J=1.7 Hz/8.1 Hz, 1H); 7.51 d (J=7.9 Hz, 1H); 7.28 d (J=2.3 Hz, 1H); 7.07 d (J=4.7 Hz, 1H); 7.03 d (J=4.5 Hz, 1H); 6.58 d (J=10.0 Hz, 1H); 5.94 d (J=10.0 Hz, 1H); 5.08 m (1H); 4.24 m (1H); 3.53 m (2H); 3.00 m (2H); 2.77 d (J=4.6 Hz, 3H); 1.43 s (3H); 1.39 s (3H).

### EXAMPLE 6

### 6-Methoxy-2-(3,4,5-trimethoxy-phenyl)-quinoline-4-carboxylic acid ((R)-2-benzo[b]-thiophen-3-yl-1-hydroxy-methyl-ethyl)-amide

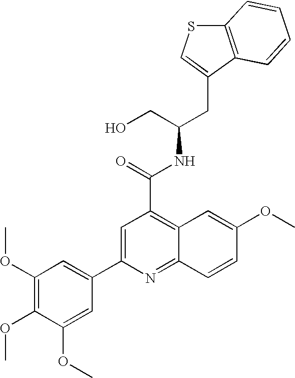

### 6a) 6-Methoxy-2-(3,4,5-trimethoxy-phenyl)-quinoline-4-carboxylic acid

3,4,5-Trimethoxy-1-acetyl benzene (2 g), 5-Methoxyisatin (2 g) and KOH (30% in water, 20 mL) were stirred under reflux overnight. The reaction mixture was diluted with water (10 times), filtered and the filtrate was acidified with glacial acetic acid at 0° C. The formed precipitate was filtered off, dried and used without further purification in the next step and yielded the title compound in 85% yield. ESI-MS [M+1]: 371.

### 6b) 0.2 mmol Wang resin (0.6 mmol/g resin loading) is swollen for 15 min in 2 mL NMP.

After filtration, the resin is reacted for 12 h with 6 eq (R)-3-Benzo[b]thiophen-3-yl-2-(9H-fluoren-9-ylmethoxycarbonylamino)-propionic acid (0.3M in NMP), 6 eq pyridine and 6 eq 2,4-Dichlorobenzoylchloride under stirring. The resin in washed with NMP (3×2 mL) and capped for 5 minutes with 2 mL 10% acetanhydride (v/v) in DMF. After washing with DMF (3×2 mL), the Fmoc group is removed by treatment with 2×2 mL 20% piperidine in DMF (2×15 min). After washing with DMF, the 6-Methoxy-2-(3,4,5-trimethoxy-phenyl)-quinoline-4-carboxylic acid (2 eq, 0.3 M in NMP) is coupled with 2 eq HATU (0.3 M in NMP) and N-Methylmorpholine (4 eq, 3M in NMP) for 4 h at room temperature to the released amine moiety. After washing with NMP (3×2 mL) and THF (3×2 mL), the alcohols are released from the resin by reduction with 2 mL 1M DIBAH in THF at 0° C. After 12 h, the resin is filtered and washed with THF (1×2 mL). 1 mL 1M KHSO₄ is added to the filtrate at room temperature, and the mixtures is evaporated to dryness. The residue is suspended in DMSO, filtered (2×2 mL), and the filtrate is subjected to preparative HPLC. HPLC purification: Machine: Analytical 4 channel MUX system with CTC Pal injector, Waters 1525 Pumps, Waters 2488 UV detector and Waters ZQ 2000 single quad MS detector. Column X-Bridge RP C18 4.6×50 3.5 μM; detection wavelength 214 nm; flow rate 1 ml/min; eluents A: 0.1% TFA in H₂O, B 0.1% TFA in ACN; gradient in each case based on B: 1% to 99% (10′) to 99% (2′) to 1% (0.5′) to 1% (3.5′). Molecular peak (ESI, M+1): 560. Retention time: 8.5 min.

The following compounds were obtained in analogy to the preparation methods described above:

| | | Method | | |
|---|---|---|---|---|
| | Product; | analogous | | |
| Ex. | reagents | to | HPLC-MS | Structure |
| 7 | 4-Ethoxy-3′-methoxy-biphenyl-3-carboxylic acid((R)-2-benzo[b]thiophen-3-yl-1-hydroxymethyl-ethyl)-amide;4-Ethoxy-3′-methoxy-biphenyl-3-carboxylic acidand(R)-3-Benzo[b]thiophen-3-yl-2-(9H-fluoren-9-ylmethoxycarbonylamino)-propionic acid | 6 | Column X-Bridge RP C184.6 × 50 3.5 μM; detectionwavelength 214 nm; flowrate 1 ml/min; eluents A:0.1% TFA in H₂O, B 0.1%TFA in ACN; gradient ineach case based on B: 1%to 99% (10′) to 99% (2′) to1% (0.5′) to 1% (3.5′).Molecular peak (ESI,M + 1): 463Retention time: 10.48 min. | 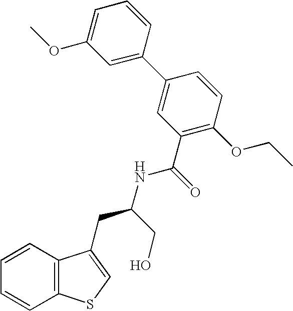 |
| 8 | 6-Methoxy-2-(3,4,5-tri-methoxy-phenyl)-quino-line-4-carboxylic acid (1-hydroxymethyl-2-quinolin-3-yl-ethyl)-amide;(R)-2-(9H-Fluoren-9-yl-methoxycarbonylamino)-3-quinolin-3-yl-propionicacid and(R)-3-Benzo[b]thiophen-3-yl-2-(9H-fluoren-9-yl-methoxycarbonylamino)-propionic acid | 6 | Column X-Bridge RP C184.6 × 50 3.5 μM; detectionwavelength 214 nm; flowrate 1 ml/min; eluents A:0.1% TFA in H₂O, B 0.1%TFA in ACN; gradient ineach case based on B: 1%to 99% (10′) to 99% (2′) to1% (0.5′) to 1% (3.5′).Molecular peak (ESI,M + 1): 555Retention time: 5.86 min. | 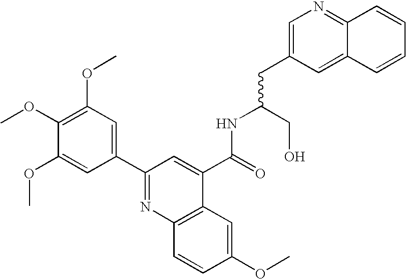 |
| 9 | 6-Methoxy-2-(3,4,5-tri-methoxy-phenyl)-quino-line-4-carboxylic acid ((R)-2-hydroxy-1-naphthalen-2-ylmethyl-ethyl)-amide;(R)-2-(9H-Fluoren-9-yl-methoxycarbonylamino)-3-naphthalen-2-yl-propionic acidand(R)-3-Benzo[b]thiophen-3-yl-2-(9H-fluoren-9-yl-methoxycarbonylamino)-propionic acid | 6 | Column X-Bridge RP C184.6 × 50 3.5 μM; detectionwavelength 214 nm; flowrate 1 ml/min; eluents A:0.1% TFA in H₂O, B 0.1%TFA in ACN; gradient ineach case based on B: 1%to 99% (10′) to 99% (2′) to1% (0.5′) to 1% (3.5′).Molecular peak (ESI,M + 1): 554Retention time: 8.06 min. | 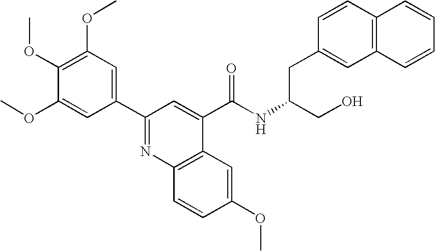 |
| 10 | 6-Iodo-2-(3,4,5-trime-thoxy-phenyl)-quinoline-4-carboxylic acid ((R)-2-hydroxy-1-naphthalen-2-ylmethyl-ethyl)-amide;(R)-2-(9H-Fluoren-9-yl-methoxycarbonylamino)-3-naphthalen-2-yl-propi-onic acidand6-Iodo-2-(3,4,5-tri-methoxy-phenyl)-quino-line-4-carboxylic acid | 6 | Column X-Bridge RP C184.6 × 50 3.5 μM; detectionwavelength 214 nm; flowrate 1 ml/min; eluents A:0.1% TFA in H₂O, B 0.1%TFA in ACN; gradient ineach case based on B: 1%to 99% (10′) to 99% (2′) to1% (0.5′) to 1% (3.5′).Molecular peak (ESI,M + 1): 649Retention time: 10.01 min. | 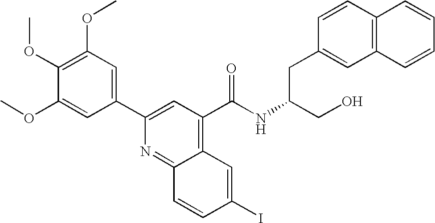 |

### EXAMPLE 11

### 6-Methoxy-2-(3,4,5-trimethoxy-phenyl)-quinoline-4-carboxylic acid (2-hydroxy-1-naphthalen-1-ylmethyl-ethyl)-amide

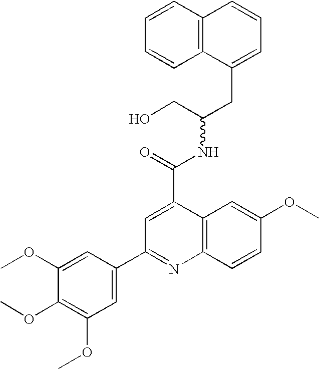

### 11a) 3-Naphthalen-1-yl-2-nitro-acrylic acid ethyl ester

To a solution of TiCl₄ (0.28 mL) in THF (14 mL) was added slowly at 0° C. Naphthalene-1-carbaldehyde (0.17 mL), Nitro-acetic acid ethyl ester (0.28 mL) and a solution of N-Methylmorpholin (0.56 mL) in THF (4 mL). The reaction mixture was allowed to warm to ambient temperature and stirred for another 90 minutes, then poured into ice water (1.5 L) and extracted with MTBE (3×). The combined organic layers were washed with water and dried over magnesium sulphate. 400 mg of the title compound was obtained after flash chromatography. ESI-MS [M+1]: 272.

### 11b) 2-Amino-3-naphthalen-1-yl-propionic acid ethyl ester

3-Naphthalen-1-yl-2-nitro-acrylic acid ethyl ester (400 mg), dissolved in MeOH (4 ml) were hydrogenated with palladium on charcoal (10%, 157 mg) at high pressure (7 bar) and ambient temperature for 5 hours. The catalyst was filtered off and the solvent was removed. The title compound was obtained in 94 mg yield after flash chromatography. ¹H-NMR (DMSO-d₆): 8.08 d (J=8.6 Hz, 1H); 7.90 d (J=7.8 Hz, 1H); 7.78 d (J=8.2 Hz, 1H); 7.50 m (2H); 7.41 m (1H); 7.32 d (J=6.6 Hz, 1H); 3.93 m (2H); 3.64 m (1H); 3.36 m (1H); 3.23 m (1H); 1.92 s (2H); 1.00 m (3H).

### 11c) 6-Methoxy-2-(3,4,5-trimethoxy-phenyl)-quinoline-4-carboxylic acid

6-Methoxy-2-(3,4,5-trimethoxy-phenyl)-quinoline-4-carboxylic acid was prepared as described in example 6a.

### 11d) 2-{[6-Methoxy-2-(3,4,5-trimethoxy-phenyl)-quinoline-4-carbonyl]-amino}-3-naphthalen-1-yl-propionic acid ethyl ester

A solution of 6-Methoxy-2-(3,4,5-trimethoxy-phenyl)-quinoline-4-carboxylic acid (143 mg), 2-Amino-3-naphthalen-1-yl-propionic acid ethyl ester (94 mg), HATU (147 mg), diisopropylethylamin (0.13 mL) in DMF (4 ml) was stirred overnight at ambient temperature. The reaction mixture was poured into saturated ammonium chloride solution and the precipitate filtered off. The title compound was obtained in 82 mg yield after flash chromatography. ESI-MS [M+1]: 595.

### 11e) 6-Methoxy-2-(3,4,5-trimethoxy-phenyl)-quinoline-4-carboxylic acid (2-hydroxy-1-naphthalen-1-ylmethyl-ethyl)-amide

To a solution of 2-{[6-Methoxy-2-(3,4,5-trimethoxy-phenyl)-quinoline-4-carbonyl]-amino}-3-naphthalen-1-yl-propionic acid ethyl ester (82 mg) in THF (2.5 ml) was added slowly a solution of lithium borohydride (2M in THF, 0.1 ml) at −10° C. The reaction mixture was stirred overnight at ambient temperature and then quenched with water (1 ml) at 0° C. The reaction mixture was neutralized with HCl (0.5 N) and extracted with ethyl acetate/water (3×). The combined organic layers were dried over magnesium sulphate, the solvent was evaporated and the title compound was obtained after flash chromatography in 60 mg yield. ¹H-NMR (DMSO-d₆): 8.77 d (J=8.6 Hz, 1H); 8.32 d (J=8.6 Hz, 1H); 8.00 d (J=9.4 Hz, 1H); 7.93 d (J=7.8 Hz, 1H); 7.90 s (1H); 7.81 d (J=8.2 Hz, 1H); 7.58 m (1H); 7.54-7.47 m (4H); 7.43 m (2H); 7.33 d (J=2.7 Hz, 1H); 5.09 m (1H); 4.48 m (1H); 3.94 s (6H); 3.91 m (1H); 3.76 s (6H); 3.72 m (1H); 3.64 m (1H); 3.51 m (1H); 3.21 m (1H).

The following compounds were obtained in analogy to the preparation methods described above:

| | | Method | | |
|---|---|---|---|---|
| | Product; | analogous | ¹H-NMR (400 MHz) | |
| Ex. | reagents | to | δ [ppm] | Structure |
| 12 | 6-Methoxy-2-(3,4,5-tri-methoxy-phenyl)-quinoline-4-carboxylic acid (2-hydroxy-1-naphthalen-2-ylmethyl-ethyl)-amide;2-{[6-Methoxy-2-(3,4,5-tri-methoxy-phenyl)-quinoline-4-carbonyl]-amino}-3-naphthalen-2-yl-propionicacid ethyl ester | 11 | ¹H-NMR (DMSO-d₆): 8.73d (J = 8.6 Hz, 1H); 7.97 d(J = 9.4 Hz, 1H); 7.91 s(1H); 7.85 m (2H); 7.79 m(2H); 7.51 d (J = 7.8 Hz,1H); 7.46 s (2H); 7.45-7.37 m (3H); 7.27 d (J =2.3 Hz, 1H); 5.02 m (1H);4.44 m (1H); 3.91 s (6H);3.75 s (3H); 3.62 m (2H);3.59 s (3H); 3.17 m (1H);2.95 m (1H). | 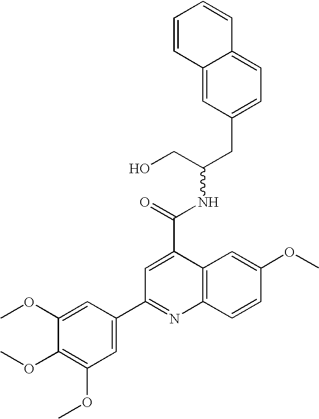 |

### EXAMPLE 13

### 3′-Chloro-4-trifluoromethoxy-biphenyl-3,4′-dicarboxylic acid 3-{[2-hydroxy-1-(4-fluoro-1H-indazol-3-ylmethyl)-ethyl]-amide}4′-methylamide

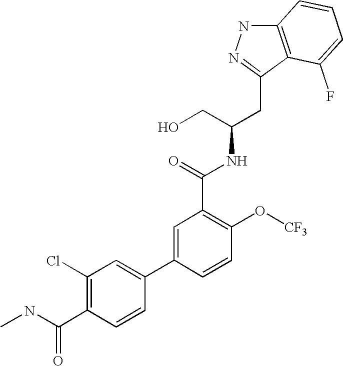

### 13a) 4-Fluoro-3-methyl-1H-indazol

The title compound was prepared in analogy to: J. Org. Chem. 71, (2006), pp. 8166-72 starting from 24.9 g 2,6-difluoroacetophenone and 150 ml hydrazine hydrate in 160 ml dimethoxyethane at reflux in 85% yield. ¹H-NMR (DMSO): 2.56 s (3H); 6.78 ddd (J=10.8 Hz/5.9 Hz/2.3 Hz, 1H); 7.24-7.30 m (2H), 12.9 s (1H).

### 13b) 2-Amino-3-(4-fluoro-1H-indazol-3-yl)-propanoic acid ethyl ester

The title compound was prepared in analogy to: Tetrahedron 62, (2006), pp. 7772-5 starting from 4-Fluoro-3-methyl-1H-indazol. The compound has not been isolated as its hydrochloride but as the pure compound after chromatography (silica, ethyl acetate/methanol). ¹H-NMR (CDCl₃): 1.27 t (J=7.1 Hz, 3H); 3.42 dd (J=14.9 Hz/8.1 Hz, 1H); 3.61 dd (J=14.9 Hz/4.7 Hz, 1H); 4.08 dd (J=8.1 Hz/4.7 Hz, 1H); 4.23 dq (J=7.1 Hz/1 Hz, 2H); 6.77 ddd (J=10.5 Hz/7.5 Hz/0.8 Hz, 1H); 7.20 d (J=8.3 Hz, 1H); 7.26 dd (J=7.5 Hz/4.9 Hz, 1H); 7.31 s (1H).

### 13c) 2-Amino-3-(4-fluoro-1H-indazol-3-yl)-propanol

To a solution of 125 mg 2-Amino-3-(4-fluoro-1H-indazol-3-yl)-propanoic acid ethyl ester in 40 ml THF and 20 ml ethanol were given 4 eq lithium borohydride and the reaction was stirred for 90 min. at RT. The resulting suspension was filtered through a pad of Celite and the solvent was evaporated in vacuum. The resulting oil was purified by flash chromatography (IST ISOLUTE® Flash NH2; hexane/ethyl acetate/methanol) and 57 mg (55%) of the title compound were obtained. ¹H-NMR (CDCl₃): 2.80 dd (J=14.2 Hz/7.9 Hz, 1H); 3.04 dd (J=14.3 Hz/5.6 Hz, 1H); 3.08-3.17 m (1H); 3.24 dd (J=10.3 Hz/6.8 Hz, 1H); 3.35 dd (J=10.1 Hz/5.5 Hz, 1H); 6.77 ddd (J=11.1 Hz/5.8 Hz/1.5 Hz, 1H); 7.24-7.31 m (1H).

### 13d) 3-Bromo-6-trifluoromethoxy-benzoic acid

A solution of 76.5 ml (4-bromo-phenyl)-trifluoromethylether in 100 ml THF were added slowly at −70° C. to a solution of 514.5 mmol LDA in 900 ml THF. The solution was kept for further 2 hours at −70° C. and was than added to a mixture of ca. 50 g dry ice given into 250 ml THF at −78° C. The temperature was kept for further 2 hours and than the temperature was allowed to rise to RT over night. The THF was removed in vacuum and the resulting crude product was diluted in diethyl ether. The organic phase was extracted with 2.5 M sodium hydroxide solution. The water phase was acidified with hydrochloric acid to a pH=1 and extracted with dichloromethane 3 times. The combined organic phases were poured into hexane and stirred for several hours. The resulting precipitate was filtered off and dried in vacuum to deliver 56.9 g of the title compound. ¹H-NMR (CDCl₃): 7.48 dq (J=8.8 Hz/1.2 Hz, 1H); 7.92 dd (J=8.8 Hz/2.6 Hz, 1H); 8.04 d (J=2.6 Hz, 1H); 13.78 bs (1H).

### 13e) 3-Bromo-6-trifluoromethoxy-benzoic acid methyl ester

A solution of 20 g 3-Bromo-6-trifluoromethoxy-benzoic acid and 2 ml conc. sulphuric acid in 200 ml methanol were stirred at reflux for 12 hours. The solvent was removed in vacuum and the resulting oil was diluted in ethyl acetate. The organic phase was washed with brine 3 times and dried with anhydrous sodium sulphate. After removal of the solvent in vacuum 20.58 g of the title compound were isolated as a yellow oil. ¹H-NMR (DMSO): 3.83 s (3H); 7.47 dq (J=8.8 Hz/1.3 Hz, 1H); 7.92 dd (J=8.8 Hz/2.6 Hz, 1H); 8.03 d (J=2.6 Hz, 1H).

### 13f) 3-Chloro-4′-trifluoromethoxy-biphenyl-3′,4-dicarboxylic acid 4-methylamide

A solution of 5 g 3-Bromo-6-trifluoromethoxy-benzoic acid methyl ester, 4.28 g 3-chloro-4-(N-methyl-aminocarbonyl)-phenylboronic acid, 4.62 g potassium carbonate and 190 mg palladium acetate in 125 ml THF and 5 ml water was heated in a sealed tube at 70° C. for 16 hours. The reaction mixture was filtered through Celite and the solution was adjusted with 1M hydrochloric acid to pH=3. After extraction with ethyl acetate the combined organic phases were washed with brine and dried with anhydrous sodium sulphate. The solvent was evaporated in vacuum and the resulting crude product was purified by flash chromatography (silica, methylene chloride) to give 4.68 g 3-Chloro-4′-trifluoromethoxy-biphenyl-3′,4-dicarboxylic acid 4-methyl amide 3-methyl ester. This was dissolved in 38 ml methanol and 19 ml water and treated with 0.9 ml sodium hydroxide solution (50%) at RT. After 6 hours the methanol was removed in vacuum and the remaining solution was extracted with a iso-propanol/methylene chloride mixture (1:4). The organic phase was washed with brine and dried with anhydrous sodium sulphate. The solvent was removed in vacuum and 4.0 g of the title compound was isolated. ¹H-NMR (DMSO): 3.32 s (3H); 7.54 d (J=7.9 Hz, 1H); 7.59 dd (J=8.6 Hz/1.3 Hz, 1H); 7.75 dd (J=7.9 Hz/1.7 Hz, 1H); 7.88 d (J=1.7 Hz, 1H); 8.04 dd (J=8.5 Hz/2.5 Hz, 1H); 8.18 d (J=2.6 Hz, 1H); 13.60 bs (1H).

### 13g) 3′-Chloro-4-trifluoromethoxy-biphenyl-3,4′-dicarboxylic acid 3-{[2-hydroxy-1-(4-fluoro-1H-indazol-3-ylmethyl)-ethyl]-amide}4′-methylamide

A solution of 85 mg 3-Chloro-4′-trifluoromethoxy-biphenyl-3′,4-dicarboxylic acid 4-methylamide, 48 mg 2-Amino-3-(4-fluoro-1H-indazol-3-yl)-propanol, 87 mg HATU, 0.076 ml N-methyl-morpholine and 7 mg DMAP in 2.8 ml THF were stirred for 20 hours at RT. The solvent was removed in vacuum and the residue was purified by HPLC chromatography (Column X-Bridge RP C18 30×100 5 μM; detection: DAD TAC 200-400 nM; flow rate 50 ml/min; eluents A: 0.1% formic acid in H₂O, B 0.1% formic in ACN; gradient) to obtain 79 mg of the title compound. ¹H-NMR (DMSO): 2.79 d (J=4.5 Hz, 3H); 3.11 dd (J=14.5 Hz/8.3 Hz, 1H); 3.27-3.40 m (2H); 3.47-3.62 m (2H); 4.39-4.53 m (1H); 4.83 t (J=5.6 Hz, 1H); 6.74-6.82 m (1H); 7.25-7.31 m (2H); 7.47 dd (J=8.7 Hz/1.3 Hz, 1H); 7.57 d (J=7.9 Hz, 1H); 7.69 dd (J=7.9 Hz/1.7 Hz, 1H); 7.72 d (J=2.5 Hz, 1H); 7.82 d (J=1.7 Hz, 1H); 7.88 dd (J=8.7 Hz/2.5 Hz, 1H); 8.33-8.45 m (2H); 12.98 s (1H).

### EXAMPLE 14

### 3′-Chloro-4-trifluoromethoxy-biphenyl-3,4′-dicarboxylic acid 3-{[2-hydroxy-1-(7-fluoro-1H-indazol-3-ylmethyl)-ethyl]-amide}4′-methylamide

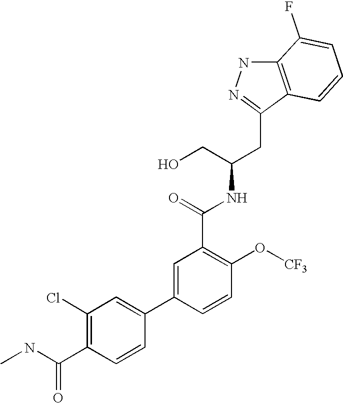

### 14a) 7-Fluoro-3-methyl-1H-indazol

The title compound was prepared in analogy to: J. Org. Chem. 71, (2006), pp. 8166-72 starting from 23.7 g 2,3-difluoroacetophenone and 150 ml hydrazine hydrate in 150 ml dimethoxyethane at reflux in about 77% yield. ¹H-NMR (DMSO): 2.50 s (3H); 7.03 dt (J=7.8 Hz/4.5 Hz, 1H); 7.15 dd (J=11.4 Hz/7.6 Hz, 1H), 7.53 d (J=8.0 Hz, 1H); 13.2 s (1H).

### 14b) 2-Amino-3-(7-fluoro-1H-indazol-3-yl)-propanoic acid ethyl ester

The title compound was prepared in analogy to: Tetrahedron 62, (2006), pp. 7772-5 starting from 7-Fluoro-3-methyl-1H-indazol. The compound has not been isolated as its hydrochloride but as the pure compound after chromatography (silica, ethyl acetate/methanol). ¹H-NMR (DMSO): 1.04 t (J=7.0 Hz, 3H); 3.12-3.28 m (2H); 3.76 dd (J=6.4 Hz/6.3 Hz, 1H); 3.97 q (J=7.2 Hz, 2H); 7.05 ddd (J=12.4 Hz/7.9 Hz/4.7 Hz, 1H); 7.16 dd (J=11.5 Hz, 7.5 Hz, 1H); 7.55 d (J=8.1 Hz, 1H); 13.30 s (1H).

### 14c) 2-Amino-3-(7-fluoro-1H-indazol-3-yl)-propanol

To a solution of 1.3 g 2-Amino-3-(7-fluoro-1H-indazol-3-yl)-propanoic acid ethyl ester in 50 ml THF and 25 ml ethanol were given 8 eq lithium borohydride and the reaction was stirred for 17 hours at RT. The resulting suspension was filtered through a pad of Celite and the solvent was evaporated in vacuum. The resulting oil was purified by flash chromatography (IST ISOLUTE® Flash NH2; hexane/ethyl acetate/methanol) and 261 mg (24%) of the title compound were obtained. ¹H-NMR (DMSO): 2.78 dd (J=13.9 Hz/7.4 Hz, 1H); 2.97-3.15 m (2H); 3.24 dd (J=10.4 Hz/6.2 Hz, 1H); 3.33 dd (J=10.2 Hz/4.9 Hz, 1H); 7.03 dt (J=7.9 Hz/4.5 Hz, 1H); 7.14 dd (J=11.5 Hz/7.9 Hz, 1H), 7.57 d (J=7.9 Hz, 1H).

### 14d) 3′-Chloro-4-trifluoromethoxy-biphenyl-3,4′-dicarboxylic acid 3-{[2-hydroxy-1-(7-fluoro-1H-indazol-3-ylmethyl)-ethyl]-amide}4′-methylamide

A solution of 75 mg 3-Chloro-4′-trifluoromethoxy-biphenyl-3′,4-dicarboxylic acid 4-methylamide, 42 mg 2-Amino-3-(7-fluoro-1H-indazol-3-yl)-propanol, 76 mg HATU, 0.066 ml N-methyl-morpholine and 6 mg DMAP in 2.5 ml THF were stirred for 20 hours at RT. The solvent was removed in vacuum and the residue was purified by HPLC chromatography (Column X-Bridge RP C18 30×100 5 μM; detection: DAD TAC 200-400 nM; flow rate 50 ml/min; eluents A: 0.1% formic acid in H₂O, B 0.1% formic in ACN; gradient) to obtain 51 mg of the title compound. ¹H-NMR (DMSO): 2.79 d (J=4.7 Hz, 3H); 3.10 dd (J=14.5 Hz/7.7 Hz, 1H); 3.23-3.42 m (2H); 3.43-3.62 m (2H); 4.28-4.41 m (1H); 4.91 t (J=5.7 Hz, 1H); 7.02 dt (J=7.9 Hz/4.5 Hz, 1H); 7.15 dd (J=11.7 Hz/7.7 Hz, 1H); 7.48 dd (J=8.7 Hz/1.5 Hz, 1H); 7.56 d (J=7.9 Hz, 1H); 7.60-7.70 m (3H); 7.80 d (J=1.7 Hz, 1H); 7.89 dd (J=8.7 Hz/2.5 Hz, 1H); 8.38-8.49 m (2H); 13.29 s (1H).

### EXAMPLE 15

### 3′-Chloro-4-trifluoromethoxy-biphenyl-3,4′-dicarboxylic acid 3-{[2-hydroxy-1-(7-trifluoromethyl-1H-indazol-3-ylmethyl)-ethyl]-amide}4′-methylamide

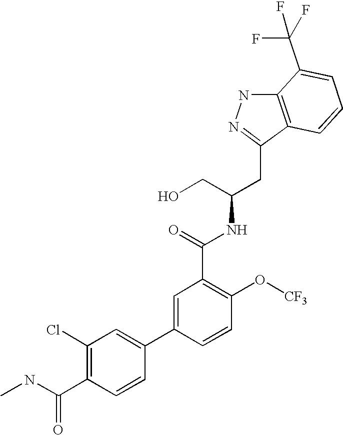

### 15a) 3-Methyl-7-trifluoromethyl-1H-indazol

The title compound was prepared in analogy to: J. Org. Chem. 71, (2006), pp. 8166-72 starting from 24.9 g 2-fluoro-3-trifluoromethyl-acetophenone and 120 ml hydrazine hydrate in 120 ml dimethoxyethane at reflux in about 62% yield. ¹H-NMR (DMSO): 2.54 (3H); 7.24 t (J=7.3 Hz, 1H); 7.71 d (J=7.3 Hz, 1H), 8.04 d (J=8.1 Hz, 1H); 13.21 s (1H).

### 15b) 2-Amino-3-(7-trifluoromethyl-1H-indazol-3-yl)-propanoic acid ethyl ester

The title compound was prepared in analogy to: Tetrahedron 62, (2006), pp. 7772-5 starting from 3-Methyl-7-trifluoromethyl-1H-indazol. The compound has not been isolated as its hydrochloride but as the pure compound after chromatography (silica, ethyl acetate/methanol). ¹H-NMR (CDCl₃): 1.22 t (J=7.2 Hz, 3H); 3.35 dd (J=14.9 Hz/7.7 Hz, 2H); 3.49 dd (J=14.7 Hz/4.7 Hz, 1H); 4.02 dd (J=7.5 Hz/4.7 Hz, 1H); 4.16 dq (J=7.2 Hz/1.9 Hz, 2H); 7.25 t (J=7.4 Hz, 1H); 7.65 d (J=7.4 Hz, 1H); 7.93 d (J=8.1 Hz, 1H).

### 15c) 2-Amino-3-(7-trifluoromethyl-1H-indazol-3-yl)-propanol

To a solution of 39.5 mg 2-Amino-3-(7-trifluoromethyl-1H-indazol-3-yl)-propanoic acid ethyl ester in 10 ml THF and 5 ml ethanol were given 8 eq lithium borohydride and the reaction was stirred for 20 hours at RT. The resulting suspension was filtered through a pad of Celite and the solvent was evaporated in vacuum. The resulting oil was dissolved in ethyl acetate and the organic phase was extracted with saturated ammonium chloride solution, water and brine. After drying over anhydrous sodium sulphate the solvent was removed in vacuum and 35.5 mg of the title compound were isolated. ¹H-NMR (DMSO): 2.83 dd (J=13.4 Hz/6.8 Hz, 1H); 3.02-3.18 m (2H); 3.23-3.41 m (2H); 7.24 t (J=7.4 Hz, 1H); 7.71 d (J=7.4 Hz, 1H), 8.10 d (J=8.1 Hz, 1H).

### 15d) 3′-Chloro-4-trifluoromethoxy-biphenyl-3,4′-dicarboxylic acid 3-{[2-hydroxy-1-(7-fluoro-1H-indazol-3-ylmethyl)-ethyl]-amide}4′-methylamide

A solution of 40 mg-Chloro-4′-trifluoromethoxy-biphenyl-3′,4-dicarboxylic acid 4-methylamide, 28 mg 2-Amino-3-(7-trifluoromethyl-1H-indazol-3-yl)-propanol, 41 mg HATU, 0.036 ml N-methyl-morpholine and 3 mg DMAP in 1.3 ml THF were stirred for 20 hours at RT. The solvent was removed in vacuum and the residue was purified by HPLC chromatography (Column X-Bridge RP C18 30×100 5 μM; detection: DAD TAC 200-400 nM; flow rate 50 ml/min; eluents A: 0.1% formic acid in H₂O, B 0.1% formic in ACN; gradient) to obtain 29 mg of the title compound. ¹H-NMR (DMSO): 2.78 d (J=4.7 Hz, 3H); 3.14 dd (J=14.3 Hz/7.9 Hz, 1H); 3.32 dd (J=14.5 Hz, 5.8 Hz, 1H); 3.45-3.6 m (2H); 4.31-4.43 m (1H); 7.22 t (J=7.5 Hz, 1H); 7.46 dd (J=8.7 Hz/1.3 Hz, 1H); 7.54 d (J=8.1 Hz, 1H); 7.58-7-67 m (2H); 7.70 d (J=7.4 Hz, 1H); 7.78 d (J=1.7 Hz, 1H); 7.87 dd (J=8.7 Hz/2.5 Hz, 1H); 8.16 d (J=8.3 Hz, 1H); 8.37-8.48 m (2H).

### EXAMPLE 16

### N-[2-hydroxy-1-(4-fluoro-1H-indazol-3-ylmethyl)-ethyl]-2-trifluoromethoxy-5-[(4-methylaminocarbonyl-phenyl)-ethynyl]-benzamide

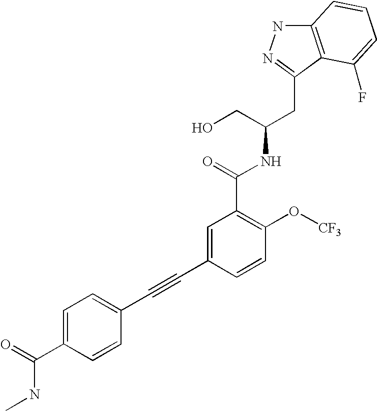

### 16a) 5-(2-Trimethylsilylethynyl)-2-trifluoromethoxy-benzoic acid methyl ester

A solution of 12.69 g 3-bromo-6-trifluoromethoxy-benzoic acid methyl ester, 25 g trimethylsilylacetylene, 1 g Palladium dba, 323 mg Copper(I)iodide and 2.23 g triphenylphosphine in 313 ml triethylamine were stirred at 60° C. for 20 hours. The reaction was filtered through a pad if Celite and treated with 450 ml hexane. The precipitate was filtered off and the solution was evaporated in vacuum. The remaining oil was purified by flash chromatography (silica, hexane/ethyl acetate gradient) and 10.1 g of the title compound were isolated. ¹H-NMR (DMSO): 0.25 s (9H); 3.86 s (3H); 7.52 dd (J=8.7 Hz/1.3 Hz, 1H); 7.81 dd (J=8.5 Hz/2.2 Hz, 1H); 7.94 d (J=2.2 Hz, 1H).

### 16b) 5-Ethynyl-2-trifluoromethoxy-benzoic acid

3.7 g-(2-Trimethylsilylethynyl)-2-trifluoromethoxy-benzoic acid methyl ester in 37 ml methanol and 18.5 ml water were treated with 0.8 ml of a sodium hydroxide solution (50%) at RT and stirred for 20 hours. The methanol was evaporated in vacuum and the remaining solution was extracted with diethyl ether. Than the water phase was treated with hydrochloric acid to adjust the pH=1. The solution was extracted with ethyl acetate, the organic phase was dried with anhydrous sodium sulphate and evaporated in vacuum to result in 2.66 g of the title compound. ¹H-NMR (DMSO): 4.42 s (1H); 7.51 dd (J=8.5 Hz/1.1 Hz, 1H); 7.79 dd (J=8.7 Hz/2.2 Hz, 1H); 7.94 d (J=2.2 Hz, 1H); 13.66 s (1H).

### 16c) 5-[(4-Methylaminocarbonyl-phenyl)-ethynyl]-2-trifluoromethoxy-benzoic acid

A solution of 125 mg 5-Ethynyl-2-trifluoromethoxy-benzoic acid, 170.2 mg N-methyl-4-iodobenzoic acid amide, 12.5 mg Palladium dba, 4.1 mg Copper(I)iodide and 28.5 mg triphenylphosphine in 3.75 ml triethylamine were stirred at 65° C. for 20 hours. The reaction was filtered through a pad if Celite and the solution was evaporated in vacuum. The remaining oil was purified by flash chromatography (silica, methylene chloride/methanol gradient) and 280 mg of the title compound were isolated as its triethylammonium salt. ¹H-NMR (DMSO): 1.18 t (J=7.3 Hz, 1H); 2.79 d (J=4.5 Hz, 1H); 3.04-3.14 m (2H); 7.56 d (J=8.6 Hz, 1H); 7.69 d (J=8.3 Hz, 1H); 7.87-7.92 m (3H); 8.07 d (J=2.0 Hz, 1H); 8.56 bq (J=4.5 Hz, 1H); 9.23 s (1H).

### N-[2-hydroxy-1-(4-fluoro-1H-indazol-3-ylmethyl)-ethyl]-2-trifluoromethoxy-5-[(4-methylaminocarbonyl-phenyl)-ethynyl]-benzamide

A solution of 73 mg 5-[(4-Methylaminocarbonyl-phenyl)-ethynyl]-2-trifluoromethoxybenzoic acid, 42 mg 2-Amino-3-(4-fluoro-1H-indazol-3-yl)-propanol, 76 mg HATU, 0.066 ml N-methyl-morpholine and 6 mg DMAP in 2.5 ml THF were stirred for 20 hours at RT. The solvent was removed in vacuum and the residue was purified by HPLC chromatography (Column X-Bridge RP C18 30×100 5 μM; detection: DAD TAC 200-400 nM; flow rate 50 ml/min; eluents A: 0.1% formic acid in H₂O, B 0.1% formic in ACN; gradient) to obtain 33 mg of the title compound. ¹H-NMR (DMSO): 2.79 d (J=4.6 Hz, 3H); 3.08 dd (J=14.7 Hz/8.3 Hz, 1H); 3.29-3.38 m (1H); 3.34-3.58 m (2H); 4.38-4.50 m (1H); 4.84 t (J=5.8 Hz, 1H); 6.77-6.85 m (1H); 7.26-7.32 m (2H); 7.45 dd (J=8.3 Hz/1.3 Hz, 1H); 7.61 d (J=2.3 Hz, 1H); 7.68 d (J=8.3 Hz, 2H); 7.73 dd (J=8.6 Hz/2.3 Hz, 1H); 7.91 d (J=8.3 Hz, 2H); 8.40 d (J=8.6 Hz, 1H); 8.56 q (J=4.6 Hz, 1H); 13.02 (1H).

### EXAMPLE 17

### N-[2-hydroxy-1-(7-fluoro-1H-indazol-3-ylmethyl)-ethyl]-2-trifluoromethoxy-5-[(4-methylaminocarbonyl-phenyl)-ethynyl]-benzamide

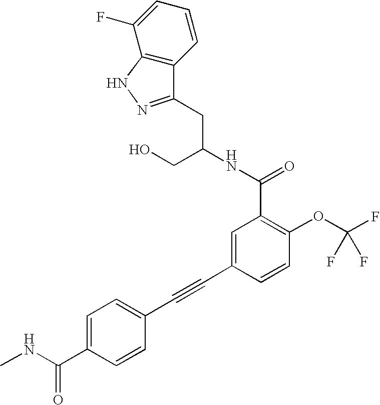

A solution of 73 mg 5-[(4-Methylaminocarbonyl-phenyl)-ethynyl]-2-trifluoromethoxybenzoic acid, 42 mg 2-Amino-3-(7-fluoro-1H-indazol-3-yl)-propanol, 76 mg HATU, 0.066 ml N-methyl-morpholine and 6 mg DMAP in 2.5 ml THF were stirred for 20 hours at RT. The solvent was removed in vacuum and the residue was purified by HPLC chromatography (Column X-Bridge RP C18 30×100 5 μM; detection: DAD TAC 200-400 nM; flow rate 50 ml/min; eluents A: 0.1% formic acid in H₂O, B 0.1% formic in ACN; gradient) to obtain 7 mg of the title compound. ¹H-NMR (DMSO): 2.80 d (J=4.5 Hz, 3H); 3.07 dd (J=14.5 Hz/8.1 Hz, 1H); 3.27 dd (J=12.4 Hz/4.0 Hz, 1H); 3.41-3.59 m (2H); 4.27-4.42 m (1H); 4.90 t (J=5.7 Hz, 1H); 7.05 dt (J=7.9 Hz/4.7 Hz, 1H); 7.15 dd (J=11.5 Hz/7.4 Hz, 1H); 7.45 dd (J=8.7 Hz/1.5 Hz, 1H); 7.52 d (J=2 Hz, 1H); 7.68 d (J=8.5 Hz, 2H); 7.73 dd (J=8.5 Hz/2.1 Hz, 1H); 7.91 d (J=8.5 Hz, 2H); 8.44 d (J=8.5 Hz, 1H); 8.56 q (J=4.5 Hz, 1H); 13.30 s (1H).

### EXAMPLE 18

### N-[2-hydroxy-1-(7-trifluoromethyl-1 indazol-3-ylmethyl)-ethyl]-2-trifluoromethoxy-5-[(4-methylaminocarbonyl-phenyl)-ethynyl]-benzamide

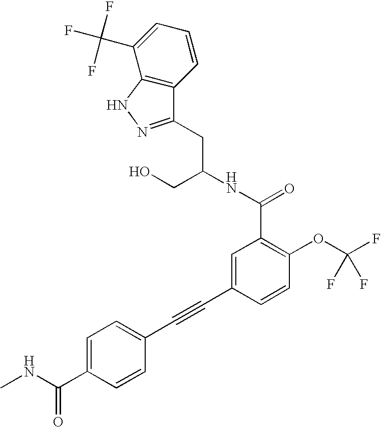

A solution of 73 mg 5-[(4-Methylaminocarbonyl-phenyl)-ethynyl]-2-trifluoromethoxybenzoic acid, 52 mg 2-Amino-3-(7-trifluoromethyl-1H-indazol-3-yl)-propanol, 76 mg HATU, 0.066 ml N-methyl-morpholine and 6 mg DMAP in 2.5 ml THF were stirred for 20 hours at RT. The solvent was removed in vacuum and the residue was purified by HPLC chromatography (Column X-Bridge RP C18 30×100 5 μM; detection: DAD TAC 200-400 nM; flow rate 50 ml/min; eluents A: 0.1% formic acid in H₂O, B 0.1% formic in ACN; gradient) to obtain 49 mg of the title compound. ¹H-NMR (DMSO): 2.80 d (J=4.3 Hz, 3H); 3.12 dd (J=14.1 Hz/7.9 Hz, 1H); 3.26-3.38 m (1H and water); 3.43-3.60 m (2H); 4.29-4.43 m (1H); 4.93 t (J=5.7 Hz, 1H); 7.25 t (J=7.7 Hz, 1H); 7.44 dd (J=8.5 Hz/1.3 Hz, 1H); 7.48 d (J=2.3 Hz, 1H); 7.67 d (J=8.3 Hz, 2H); 7.73 dd (J=8.7 Hz/2.1 Hz, 1H); 7.91 d (J=8.3 Hz, 2H); 8.14 d (J=8.1 Hz, 1H); 8.45 (J=8.5 Hz, 1H); 8.56 q (J=4.3 Hz, 1H); 13.30 s (1H).

### EXAMPLE 19

### 6-Bromo-2-(3,4,5-trimethoxy-phenyl)-quinoline-4-carboxylic acid [2-hydroxy-1-(1H-pyrrolo[3,2-b]pyridin-3-ylmethyl)-ethyl]-amide

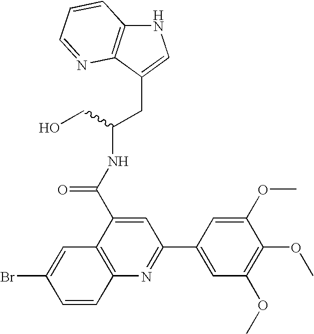

### 19a) 6-Bromo-2-(3,4,5-trimethoxy-phenyl)-quinoline-4-carboxylic acid

The title compound was prepared in analogy to the procedure described in example 6a. ESI-MS [M+1]: 419.

### 19b) Dimethyl-(1H-pyrrolo[3,2-b]pyridin-3-ylmethyl)-amine

To the mixture of dimethylamine hydrochloride (8.8 g) in isopropanol (400 mL) paraformaldehyde (3.3 g) was added. The mixture was stirred for 10 min and 4-azaindole (11.8 g) was added. This mixture was heated for 1.5 h at 80° C. After cooling the mixture was filtered and concentrated under reduced pressure. The residue was diluted with water (100 ml) and HCl (10 mL), washed with ethyl ether and concentrated under reduced pressure. The residue was dissolved in 30% NaOH and extracted with ethyl ether. Organic phases were collected, dried (K₂CO₃) and concentrated under reduced pressure to give the title compound (10 g).

### 19c) 2-Acetylamino-2-(1H-pyrrolo[3,2-b]pyridin-3-ylmethyl)-malonic acid diethyl ester

The mixture of Dimethyl-(1H-pyrrolo[3,2-b]pyridin-3-ylmethyl)-amine (3.5 g), diethyl acetamidomalonate (4.34 g), NaOH (250 mg), xylenes (35 mL) was refluxed for 5 h under Ar. The hot mixture was filtered and cooled. The precipitate formed was filtered, washed with hexane and dried to give the title compound (3.5 g).

### 19d) 2-Acetylamino-3-(1H-pyrrolo[3,2-b]pyridin-3-yl)-propionic acid ethyl ester

To the solution of 2-Acetylamino-2-(1H-pyrrolo[3,2-b]pyridin-3-ylmethyl)-malonic acid diethyl ester (3.5 g) in ethanol (40 mL) the solution of KOH (0.72 g) in water (4 mL) was added. The mixture was stirred at rt for 3 h, acidified with HCl (1 mL) and concentrated at 30° C. under reduced pressure. Water (10 mL) was added and the mixture was acidified to pH 1 and concentrated under reduced pressure. The residue was heated at 130° C. under Ar for 1 h. After cooling the residue was dissolved in water (minimum) and basified with saturated K₂CO₃. The solid formed was filtered, washed with ethyl ether and dried to give the title compound (2.5 g).

### 19e) N-[1-Hydroxymethyl-2-(1H-pyrrolo[3,2-b]pyridin-3-yl)-ethyl]-acetamide

The suspension of 2-Acetylamino-3-(1H-pyrrolo[3,2-b]pyridin-3-yl)-propionic acid ethyl ester (2.75 g), LiBH₄ (0.9 g), ethyl ether (20 mL) was stirred for 24 h at rt. The reaction was quenched at −20° C. with cooled MeOH and stirred for 3 h. Reaction mixture was acidified with KHSO₄ to pH 3 and concentrated under reduced pressure. The residue was dissolved with saturated K₂CO₃ and extracted with acetonitrile. Organic phase was dried and concentrated to give the title compound (1.2 g).

### 19f) 2-Amino-3-(1H-pyrrolo[3,2-b]pyridin-3-yl)-propan-1-ol

N-[1-Hydroxymethyl-2-(1H-pyrrolo[3,2-b]pyridin-3-yl)-ethyl]-acetamide (1 g) in 20% HCl (1 mL) was stirred overnight at rt. The resulting mixture was heated at 60° C. for 4 h. After concentration the residue was crystallized from methanol/chloroform (1:4) yielding the title compound (1.25 g) as dihydrochloride salt. ESI-MS [M+1]: 192.

### 19g) 6-Bromo-2-(3,4,5-trimethoxy-phenyl)-quinoline-4-carboxylic acid [2-hydroxy-1-(1H-pyrrolo[3,2-b]pyridin-3-ylmethyl)-ethyl]-amide

A solution of 6-Bromo-2-(3,4,5-trimethoxy-phenyl)-quinoline-4-carboxylic acid (109 mg), 2-Amino-3-(1H-pyrrolo[3,2-b]pyridin-3-yl)-propan-1-ol dihydrochloride (60 mg), HOBt×water (48 mg) and EDCI (60 mg), triethylamine (0.1 ml) in DMF (3 ml) was stirred overnight at ambient temperature. The reaction mixture was poured into water, extracted with ethyl acetate (3×). The combined organic layers were washed with brine and the solvent removed under vacuum. The title compound was obtained in 3% yield after flash chromatography. ¹H-NMR (DMSO-d₆): 11.07 s (1H); 8.06 d (J=8.1 Hz, 1H); 8.15 d (J=2.3 Hz, 1H); 8.11 s (1H); 8.07 dd (J=1.5 Hz/4.8 Hz, 1H); 8.01 d (J=8.8 Hz, 1H); 7.87 dd (J=2.3 Hz/9.1 Hz, 1H); 7.70 dd (J=1.3 Hz/8.1 Hz, 1H); 7.53 s (2H); 7.50 d (J=2.3 Hz, 1H); 7.00 dd (J=4.6 Hz/8.1 Hz, 1H); 5.10 s (1H); 4.39 m (1H); 3.52 m (2H); 3.14 m (1H); 3.05 m (1H).

The following compounds were obtained in analogy to the preparation methods described above:

| | | Method | | |
|---|---|---|---|---|
| | Product; | analogous | ¹H-NMR (400 MHz) | |
| Ex. | reagents | to | δ [ppm] | Structure |
| 20 | 2-(7-Methoxy-benzofuran-2-yl)-6-trifluoromethoxy-quinoline-4-carboxylic acid[2-hydroxy-1-(1H-pyrrolo[3,2-b]pyridin-3-ylmethyl)-ethyl]-amide;2-Amino-3-(1H-pyrrolo[3,2-b]pyridin-3-yl)-propan-1-oland2-(7-Methoxy-benzofuran-2-yl)-6-trifluoromethoxy-quinoline-4-carboxylic acid | 19 | ¹H-NMR (DMSO-d₆):11.27 s (1H); 9.09 d (J =7.9 Hz, 1H); 8.27 m (2H);8.19 s (1H); 7.88 m (4H);7.60 s (1H); 7.38 d (J =7.7 Hz, 1H); 7.30 m (1H);7.11 m (2H); 5.16 s (1H);4.48 m (1H); 4.03 s (3H);3.60 d (J = 5.5 Hz, 2H);3.17 m (1H); 3.10 m (1H). | 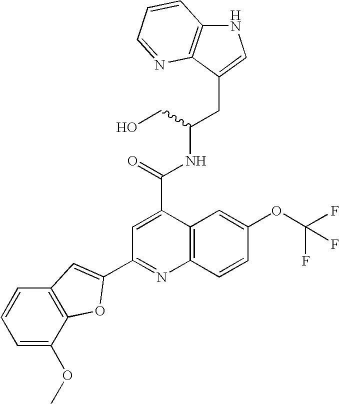 |

### EXAMPLE 21

### 2-(3-Fluoro-4-methoxy-phenyl)-6-methoxy-quinazoline-4-carboxylic acid [2-hydroxy-1-(1H-pyrrolo[3,2-b]pyridin-3-ylmethyl)-ethyl]-amide

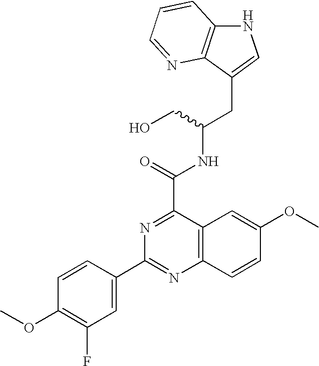

### 21a) 2-Amino-3-(1H-pyrrolo[3,2-b]pyridin-3-yl)-propan-1-ol

The title compound was prepared as described in example 19b-19f.

### 21b) 5-Methoxyisatin sodium salt

A solution of 1N KOH (100 ml) was slowly added in portions to a suspension of 5-methoxyisatin (17.7 g) in water (100 ml), and the mixture was heated to about 40° C. It was stirred until almost all the isatin had dissolved. The undissolved residue was filtered off, and the filtrate was evaporated to dryness in a rotary evaporator. Absolute ethanol (200 ml) was added to the residue, and the solid was stirred at room temperature, and the sodium salt of 5-methoxyisatin was filtered off and dried in vacuo at room temperature. Yield 20.6 g (96%).

### 21c) [2-(3-Fluoro-4-methoxybenzoylamino)-5-methoxyphenyl]oxoacetic acid

Dimethylaminopyridine (3.5 g), and then triethylamine (75 ml) and subsequently a solution of 3-fluoro-4-methoxybenzoyl chloride (37.7 g) in THF (200 ml) were added dropwise to a solution of the sodium salt of 5-methoxyisatin (21.5 g) in THF (300 ml), and the reaction mixture was stirred at room temperature for 20 hours. Water (30 ml) was added to the reaction mixture and stirred for a 4 hours. The insoluble residue was filtered off and the filtrate was evaporated to dryness. The residue was again dissolved in water (900 ml) and acidified to pH 1 with 1N HCl. The precipitate which separated out was filtered off, washed with water and dried in air. Recrystallization from benzene yielded 11.1 g (32%) of the title compound. MS (ESI,+): 348 (M+1).

### 21d) 2-(3-Fluoro-4-methoxyphenyl)-6-methoxyquinazoline-4-carboxylic acid

Anhydrous ammonia (5 g) was added to a solution of [2-(3-fluoro-4-methoxybenzoylamino)-5-methoxyphenyl]oxoacetic acid (3.47 g) in ethanol (50 ml). The reaction mixture was heated in a sealed tube at 120° C. under autogenous conditions for 6 hours. Solvent and ammonia were distilled out in a rotary evaporator, and the dry residue was suspended in water (100 ml) and acidified to pH 3-4 with acetic acid. The resulting precipitate was filtered off, washed with water and recrystallized from ethanol in an autoclave at 150° C. Yield 2.1 g (65%). MS (ESI,+): 329 (M+1).

### 21e) 2-(3-Fluoro-4-methoxy-phenyl)-6-methoxy-quinazoline-4-carboxylic acid [2-hydroxy-1-(1H-pyrrolo[3,2-b]pyridin-3-ylmethyl)-ethyl]-amide

A solution of 2-(3-Fluoro-4-methoxyphenyl)-6-methoxyquinazoline-4-carboxylic acid (40 mg), 2-Amino-3-(1H-pyrrolo[3,2-b]pyridin-3-yl)-propan-1-ol dihydrochloride (24 mg), HOBt×water (19 mg) and EDCI (24 mg), triethylamine (0.03 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 3% yield after flash chromatography. ¹H-NMR (DMSO-d₆): 11.08 s (1H); 9.20 d (J=8.6 Hz, 1H); 8.31 m (2H); 8.12 d (J=3.0 Hz, 1H); 8.10 dd (J=1.5 Hz/4.8 Hz, 1H); 7.96 d (J=9.4 Hz, 1H); 7.66 m (2H); 7.53 d (J=2.5 Hz, 1H); 7.35 m (1H); 6.98 m (1H); 5.12 s (1H); 4.37 m (1H); 3.93 s (3H); 3.82 s (3H); 3.59 m (1H); 3.51 m (1H); 3.18 m (1H); 3.12 m (1H).

### EXAMPLE 22

### N-[1-Hydroxymethyl-2-(1H-pyrrolo[3,2-b]pyridin-3-yl)-ethyl]-2-isopropoxy-5-(2-methoxy-phenylethynyl)-benzamide

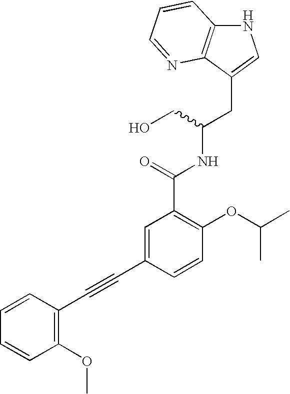

### 22a) 2-Amino-3-(1H-pyrrolo[3,2-b]pyridin-3-yl)-propan-1-ol

The title compound was prepared as described in example 19b-19f.

### 22b) 2-Isopropoxy-5-(2-methoxy-phenylethynyl)-benzoic acid

The title compound was prepared according to the procedures described in example 3a-3c.

### 22c) N-[1-Hydroxymethyl-2-(1H-pyrrolo[3,2-b]pyridin-3-yl)-ethyl]-2-isopropoxy-5-(2-methoxy-phenylethynyl)-benzamide

A solution of 2-Isopropoxy-5-(2-methoxy-phenylethynyl)-benzoic acid (39 mg), 2-Amino-3-(1H-pyrrolo[3,2-b]pyridin-3-yl)-propan-1-ol dihydrochloride (24 mg), HOBt×water (19 mg) and EDCI (24 mg), triethylamine (0.03 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 24% yield after flash chromatography. ¹H-NMR (DMSO-d₆): 11.09 s (1H); 8.33 d (J=8.3 Hz, 1H); 8.23 dd (J=1.5 Hz/4.6 Hz, 1H); 7.89 d (J=2.5 Hz, 1H); 7.70 dd (J=8.1 Hz/1.3 Hz, 1H); 7.53 dd (J=8.6 Hz/2.3 Hz, 1H); 7.45 m (2H); 7.34 m (1H); 7.16 d (J=8.8 Hz, 1H); 7.06 m (2H); 6.93 m (1H); 5.35 s (1H); 4.75 m (1H); 4.29 m (1H); 3.82 s (3H); 3.44 m (1H); 3.32 m (1H); 3.02 m (2H); 1.18 m (6H).

The following compounds were obtained in analogy to the preparation methods described above:

| | | Method | | |
|---|---|---|---|---|
| | Product; | analogous | ¹H-NMR (400 MHz) | |
| Ex. | reagents | to | δ [ppm] | Structure |
| 23 | N-[1-Hydroxymethyl-2-(1H-pyrrolo[3,2-b]pyridin-3-yl)-ethyl]-2-isopropoxy-5-phenylethynyl-benzamide;2-Isopropoxy-5-(phenyl-ethynyl)-benzoic acidand2-Amino-3-(1H-pyrrolo[3,2-b]pyridin-3-yl)-propan-1-ol | 22 | ¹H-NMR (DMSO-d₆):11.10 s (1H), 8.31 d (J =8.3 Hz, 1H); 8.23 dd (J =1.5 Hz/4.8 Hz, 1H); 7.92d (J = 2.3 Hz, 1H); 7.70dd (J = 8.1 Hz/1.3 Hz,1H); 7.57 dd (J = 8.6 Hz/2.3 Hz, 1H); 7.51 m (2H);7.45 d (J = 2.0 Hz, 1H);7.38 m (3H); 7.17 d (J =8.8 Hz, 1H); 7.06 m (1H);5.35 s (1H); 4.75 m (1H);4.29 m (1H); 3.43 m (1H);3.32 m (1H); 3.04 m (2H);1.19 m (6H). | 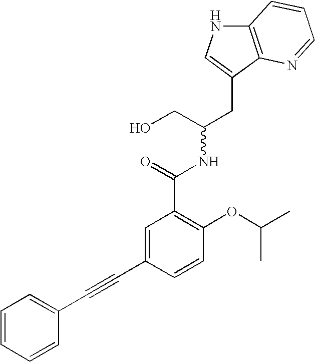 |

### EXAMPLE 24

### 5′-Fluoro-4-isopropoxy-biphenyl-3,3′-dicarboxylic acid 3-{[1-hydroxymethyl-2-(1H-pyrrolo[3,2-b]pyridin-2-yl)-ethyl]-amide}3′-methyl amide

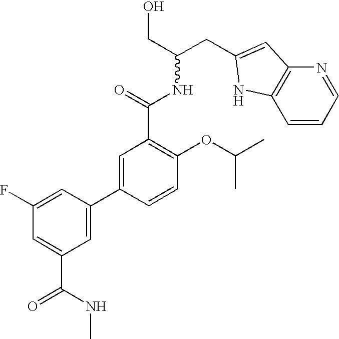

### 24a) 2-Amino-3-(1H-pyrrolo[3,2-b]pyridin-3-yl)-propan-1-ol

The title compound was prepared as described in example 19b-19f.

### 24b) 3′-Fluoro-4-isopropoxy-5′-methylcarbamoyl-biphenyl-3-carboxylic acid

The title compound was prepared according to the procedures described in example 1a-1c.

### 24c) 5′-Fluoro-4-isopropoxy-biphenyl-3,3′-dicarboxylic acid 3-{[1-hydroxymethyl-2-(1H-pyrrolo[3,2-b]pyridin-2-yl)-ethyl]-amide}3′-methylamide

A solution of 3′-Fluoro-4-isopropoxy-5′-methylcarbamoyl-biphenyl-3-carboxylic acid (41 mg), 2-Amino-3-(1H-pyrrolo[3,2-b]pyridin-3-yl)-propan-1-ol dihydrochloride (24 mg), HOBt×water (19 mg) and EDCI (24 mg), triethylamine (0.03 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 5% yield after flash chromatography. ¹H-NMR (DMSO-d₆): 11.10 s (1H); 8.66 m (1H); 8.37 d (J=8.3 Hz, 1H); 8.22 dd (J=4.6 Hz/1.3 Hz, 1H); 8.17 d (J=2.5 Hz, 1H); 7.92 m (1H); 7.80 dd (J=8.6 Hz/2.5 Hz, 1H); 7.70 dd (J=8.1 Hz/1.5 Hz, 1H); 7.60 m (1H); 7.55 m (1H); 7.47 d (J=2.3 Hz, 1H); 7.24 d (J=8.8 Hz, 1H); 7.05 m (1H); 5.37 s (1H); 4.78 m (1H); 4.32 m (1H); 3.47 m (1H); 3.34 s (1H); 3.04 m (2H); 2.78 d (J=4.6 Hz, 3H); 1.19 m (6H).

The following compounds were obtained in analogy to the preparation methods described above:

| | | Method | | |
|---|---|---|---|---|
| | Product; | analogous | ¹H-NMR (400 MHz) | |
| Ex. | reagents | to | δ [ppm] | Structure |
| 25 | 3′-Chloro-4-isopropoxy-biphenyl-3,4′-dicarboxylicacid 3-{[1-hydroxymethyl-2-(1H-pyrrolo[3,2-b]pyridin-3-yl)-ethyl]-amide}4′-methylamide;2-Amino-3-(1H-pyrrolo[3,2-b]pyridin-3-yl)-propan-1-oland3′-Chloro-4-isopropoxy-4′-methylcarbamoyl-biphenyl-3-carboxylic acid | 24 | ¹H-NMR (DMSO-d₆):11.11 s (1H); 8.36 m(2H); 8.22 dd (J = 1.3 Hz/4.6 Hz, 1H); 8.09 d (J =2.8 Hz, 1H); 7.77 dd (J =8.6 Hz/2.5 Hz, 1H); 7.70dd (J = 8.1 Hz/1.3 Hz,1H); 7.68 d (J = 1.8 Hz,1H); 7.59 d (J = 8.1 Hz/1.8 Hz, 1H); 7.45 m (2H);7.23 d (J = 8.8 Hz, 1H);7.05 m (1H); 5.36 s (1H);4.77 m (1H); 4.31 m (1H);3.46 m (1H); 3.38 m (1H);3.05 m (2H); 2.73 d (J =4.6 Hz, 3H); 1.19 m (6H). | 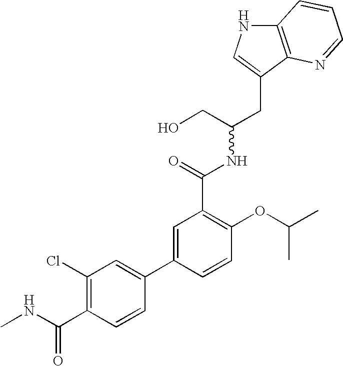 |

### EXAMPLE 26

### 7-(3,4-Dimethoxy-phenyl)-2,3,4,5-tetrahydro-benzo[b]oxepine-9-carboxylic acid [1-hydroxymethyl-2-(1H-pyrrolo[3,2-b]pyridin-3-yl)-ethyl]-amide

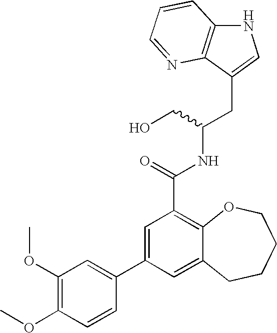

### 26a) 2-Amino-3-(1H-pyrrolo[3,2-b]pyridin-3-yl)-propan-1-ol

The title compound was prepared as described in example 19b-19f.

### 26b) 2-Allyloxy-5-bromo-benzoic acid methyl ester

The title compound was prepared from 2-Hydroxy-5-bromo-benzoic acid methyl ester in analogy to the described literature procedure via an O-alkylation. See Eur. J. Med. Chem. 1997, 32, page 385.

### 26c) 3-Allyl-5-bromo-2-hydroxy-benzoic acid methyl ester

The title compound was prepared via a Claisen rearrangement from 2-Allyloxy-5-bromobenzoic acid methyl ester in analogy to the described literature procedure. See J. Med. Chem. 1992, 35, page 310.

### 26d) 3-Allyl-2-allyloxy-5-bromo-benzoic acid methyl ester

The title compound was prepared from 3-Allyl-5-bromo-2-hydroxy-benzoic acid methyl ester in analogy to the described literature procedure via an O-alkylation. See Eur. J. Med. Chem. 1997, 32, page 385.

### 26e) 7-Bromo-2,5-dihydro-benzo[b]oxepine-9-carboxylic acid methyl ester

The title compound was prepared from 3-Allyl-2-allyloxy-5-bromo-benzoic acid methyl ester in analogy to the described literature procedure via an olefin metathesis reaction. See Heterocycles 2002, 57, page 1997.

### 26f) 7-Bromo-2,3,4,5-tetrahydro-benzo[b]oxepine-9-carboxylic acid methyl ester

The title compound was prepared from 7-Bromo-2,5-dihydro-benzo[b]oxepine-9-carboxylic acid methyl ester in analogy to the described literature procedure via a hydrogenation reaction. See Org. Lett. 2006, 15, page 3279.

### 26g) 7-(3,4-Dimethoxy-phenyl)-2,3,4,5-tetrahydro-benzo[b]oxepine-9-carboxylic acid methyl ester

The title compound was prepared in analogy to the procedure described in example 5c. ESI-MS [M+1]: 343.

### 26h) 7-(3,4-Dimethoxy-phenyl)-2,3,4,5-tetrahydro-benzo[b]oxepine-9-carboxylic acid

The title compound was prepared in analogy to the procedure described in example 5d. ESI-MS [M+1]: 329.

### 26i) 7-(3,4-Dimethoxy-phenyl)-2,3,4,5-tetrahydro-benzo[b]oxepine-9-carboxylic acid [1-hydroxymethyl-2-(1H-pyrrolo[3,2-b]pyridin-3-yl)-ethyl]-amide

A solution of 7-(3,4-Dimethoxy-phenyl)-2,3,4,5-tetrahydro-benzo[b]oxepine-9-carboxylic acid (41 mg), 2-Amino-3-(1H-pyrrolo[3,2-b]pyridin-3-yl)-propan-1-ol dihydrochloride (30 mg), HOBt×water (19 mg) and EDCI (24 mg), triethylamine (0.03 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 10% yield after flash chromatography and HPLC purification. ¹H-NMR (DMSO-d₆): 11.07 s (1H); 8.39 d (J=8.1 Hz, 1H); 8.22 dd (J=1.3 Hz/4.6 Hz, 1H); 7.73 d (J=2.5 Hz, 1H); 7.69 dd (J=1.3 Hz/8.1 Hz, 1H); 7.56 d (J=2.5 Hz, 1H); 7.47 d (J=2.3 Hz, 1H); 7.10 m (2H); 7.07 m (1H); 6.99 d (J=8.1 Hz, 1H); 5.23 s (1H); 4.28 m (1H); 3.80 s (3H); 3.75 (3H); 3.47 m (1H); 3.37 m (1H); 3.05 m (2H); 2.82 m (2H); 2.49 m (2H); 1.85 m (2H); 1.63 m (2H).

### EXAMPLE 27

### N-[1-Hydroxymethyl-2-(1H-pyrrolo[3,2-c]pyridin-3-yl)-ethyl]-2-isopropoxy-5-(2-methoxy-phenylethynyl)-benzamide

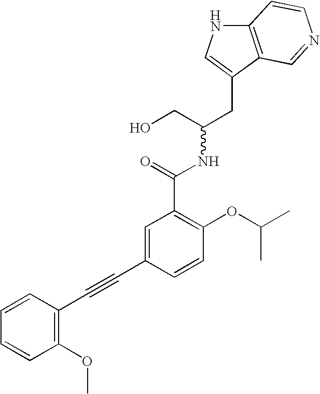

### 27a) Dimethyl-(1H-pyrrolo[3,2-c]pyridin-3-ylmethyl)-amine

To the mixture of dimethylamine hydrochloride (8.8 g) in 1-butanol (400 mL) paraformaldehyde (3.3 g) was added. The mixture was stirred for 10 min and 5-azaindole (11.8 g) was added. This mixture was refluxed for 30 min. After cooling the mixture was filtered and concentrated under reduced pressure. The residue was diluted with water (100 ml) and HCl (10 mL), washed with ethyl ether and concentrated under reduced pressure. The residue was dissolved in 30% NaOH and extracted with ethyl ether. Organic phases were collected, dried (K₂CO₃) and concentrated under reduced pressure to give the title compound (10 g).

### 27b) 2-Acetylamino-2-(1H-pyrrolo[3,2-c]pyridin-3-ylmethyl)-malonic acid diethyl ester

The mixture of Dimethyl-(1H-pyrrolo[3,2-c]pyridin-3-ylmethyl)-amine (2.1 g), diethyl acetamidomalonate (3 g), tri-n-butylphosphine (1 g, 1.3 mL) in acetonitrile (14 mL) was refluxed for 10 h under Ar. After cooling to rt water (50 mL) was added and the resulting mixture was left in the freezer overnight at 4° C. The precipitate formed was filtered and dried to give the title compound (2 g).

### 27c) 2-Acetylamino-3-(1H-pyrrolo[3,2-c]pyridin-3-yl)-propionic acid ethyl ester

To the solution of 2-Acetylamino-2-(1H-pyrrolo[3,2-c]pyridin-3-ylmethyl)-malonic acid diethyl ester (3.47 g) in ethanol (20 mL) the solution of KOH (0.6 g) in ethanol (10 mL) was added. The mixture was stirred at rt for 3 h and concentrated at 30° C. under reduced pressure. Water (10 mL) was added to the residue and resulting mixture was extracted with CH₂Cl₂ (3×10 mL). Aqueous phase was acidified to pH 1 and concentrated under reduced pressure. The residue was heated at 130° C. under Ar for 1 h. After cooling the residue was dissolved in water (minimum) and basified with saturated K₂CO₃. The solid formed was filtered, washed with ethyl ether and dried to give the title compound (1.7 g).

### 27d) N-[1-Hydroxymethyl-2-(1H-pyrrolo[3,2-c]pyridin-3-yl)-ethyl]-acetamide

The suspension of 2-Acetylamino-3-(1H-pyrrolo[3,2-c]pyridin-3-yl)-propionic acid ethyl ester (0.1 g), LiBH₄ (0.4 g), ethyl ether (10 mL), methanol (1 ml) was stirred for 5 h at rt. Then water (2 mL) was added and reaction mixture was stirred additional 2 h. Reaction mixture was acidified with KHSO₄ to pH 3 and concentrated under reduced pressure. The residue was dissolved with saturated K₂CO₃ and extracted with acetonitrile. Organic phase was dried and concentrated to give the title compound (0.08 g).

### 27e) 2-Amino-3-(1H-pyrrolo[3,2-c]pyridin-3-yl)-propan-1-ol

N-[1-Hydroxymethyl-2-(1H-pyrrolo[3,2-c]pyridin-3-yl)-ethyl]-acetamide (0.1 g) in 30% HCl (1 mL) was stirred overnight at rt. The resulting mixture was heated at 60° C. for 4 h. After concentration the residue was crystallized from methanol/chloroform (1:4) yielding the title compound (0.08 g).

### 27f) 2-Isopropoxy-5-(2-methoxy-phenylethynyl)-benzoic acid

The title compound was prepared according to the procedures described in example 3a-3c.

### 27g) N-[1-Hydroxymethyl-2-(1H-pyrrolo[3,2-c]pyridin-3-yl)-ethyl]-2-isopropoxy-5-(2-methoxy-phenylethynyl)-benzamide

A solution of 2-Isopropoxy-5-(2-methoxy-phenylethynyl)-benzoic acid (100 mg), 2-Amino-3-(1H-pyrrolo[3,2-c]pyridin-3-yl)-propan-1-ol dihydrochloride (68 mg), HOBt×water (54 mg) and EDCI (68 mg), triethylamine (0.061 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 26% yield after flash chromatography and HPLC purification. ¹H-NMR (MeOD): 9.22 s (1H); 8.52 s (1H); 8.27 d (J=6.7 Hz, 1H); 8.05 d (J=2.3 Hz, 1H); 7.76 d (J=6.4 Hz, 1H); 7.60 m (2H); 7.45 dd (J=1.7 Hz/7.5 Hz, 1H); 7.34 m (1H); 7.19 d (J=8.7 Hz, 1H); 7.05 d (J=8.05 Hz, 1H); 6.97 m (1H); 4.87 m (1H); 4.47 m (1H); 3.94 s (3H); 3.74 m (2H); 3.30 m (2H); 1.39 m (6H).

The following compounds were obtained in analogy to the preparation methods described above:

| | | Method | | |
|---|---|---|---|---|
| | Product; | analogous | ¹H-NMR (400 MHz) | |
| Ex. | reagents | to | δ [ppm] | Structure |
| 28 | N-[1-Hydroxymethyl-2-(1H-pyrrolo[3,2-c]pyridin-3-yl)-ethyl]-2-isopropoxy-5-phenylethynyl-benzamide;2-Amino-3-(1H-pyrrolo[3,2-c]pyridin-3-yl)-propan-1-oland2-Isopropoxy-5-(phenyl-ethynyl)-benzoic acid | 27 | ¹HNMR (DMSO-d₆):11.22 s (1H); 8.91 s (1H);8.33 d (J = 8.3 Hz, 1H);8.10 m (1H); 7.96 d (J =2.3 Hz, 1H); 7.60 dd (J =2.3 Hz/8.5 Hz, 1H); 7.51m (2H); 7.38 m (3H); 7.29d (J = 0.9 Hz/5.7 Hz,1H); 7.20 m (2H); 4.77 m(1H); 4.25 m (1H); 3.45 m(2H); 3.01 m (2H); 1.20 m(6H). | 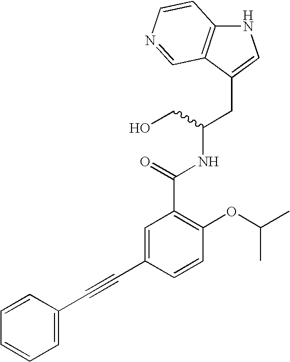 |

### EXAMPLE 29

### 2-(3-Fluoro-4-methoxy-phenyl)-6-methoxy-quinazoline-4-carboxylic acid [2-hydroxy-1-(1H-pyrrolo[3,2-c]pyridin-3-ylmethyl)-ethyl]-amide

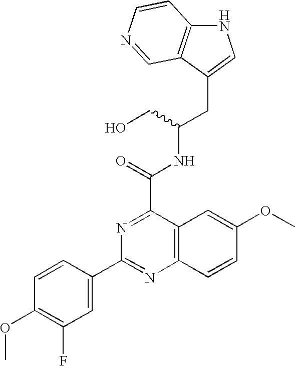

### 29a) 2-(3-Fluoro-4-methoxyphenyl)-6-methoxyquinazoline-4-carboxylic acid

The title compound was prepared according to the procedure described in the example 21b-21d.

### 29b) 2-Amino-3-(1H-pyrrolo[3,2-c]pyridin-3-yl)-propan-1-ol

The title compound was prepared according to the procedure described in the example 27a-27e.

### 29c) 2-(3-Fluoro-4-methoxy-phenyl)-6-methoxy-quinazoline-4-carboxylic acid [2-hydroxy-1-(1H-pyrrolo[3,2-c]pyridin-3-ylmethyl)-ethyl]-amide

A solution of 2-(3-Fluoro-4-methoxyphenyl)-6-methoxyquinazoline-4-carboxylic acid (85 mg), 2-Amino-3-(1H-pyrrolo[3,2-c]pyridin-3-yl)-propan-1-ol dihydrochloride (60 mg), HOBt×water (48 mg) and EDCI (60 mg), triethylamine (0.053 ml) in DMF (3 ml) was stirred overnight at ambient temperature. The reaction mixture was poured into water, extracted with ethyl acetate (3×). The combined organic layers were washed with brine and the solvent removed under vacuum. The title compound was obtained in 3% yield after flash chromatography and HPLC purification. ¹H-NMR (DMSO-d₆): 11.27 s (1H); 8.97 m (2H); 8.35 m (2H); 8.11 d (J=5.8 Hz, 1H); 8.08 d (J=3.0 Hz, 1H); 8.02 d (J=9.2 Hz, 1H); 7.71 dd (J=2.8 Hz/9.2 Hz, 1H); 7.38 m (1H); 7.32 m (2H); 5.04 m (1H); 4.44 m (1H); 3.97 s (3H); 3.86 s (3H); 3.66 m (2H); 3.22 m (2H).

### EXAMPLE 30

### 6-Bromo-2-(3,4,5-trimethoxy-phenyl)-quinoline-4-carboxylic acid [2-hydroxy-1-(1H-pyrrolo[3,2-c]pyridin-3-ylmethyl)-ethyl]-amide

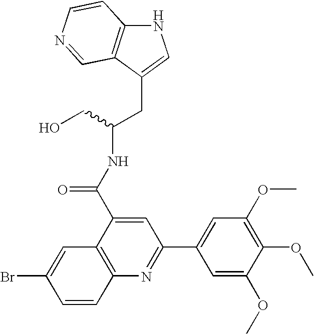

### 30a) 6-Bromo-2-(3,4,5-trimethoxy-phenyl)-quinoline-4-carboxylic acid

The title compound was prepared in analogy to the procedure described in example 6a. ESI-MS [M+1]: 419.

### 30b) 2-Amino-3-(1H-pyrrolo[3,2-c]pyridin-3-yl)-propan-1-ol

The title compound was prepared according to the procedure described in the example 27a-27e.

### 30c) 6-Bromo-2-(3,4,5-trimethoxy-phenyl)-quinoline-4-carboxylic acid [2-hydroxy-1-(1H-pyrrolo[3,2-c]pyridin-3-ylmethyl)-ethyl]-amide

A solution of 6-Bromo-2-(3,4,5-trimethoxy-phenyl)-quinoline-4-carboxylic acid (100 mg), 2-Amino-3-(1H-pyrrolo[3,2-c]pyridin-3-yl)-propan-1-ol dihydrochloride (50 mg), HOBt×water (40 mg) and EDCI (50 mg), triethylamine (0.045 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 16% yield after flash chromatography and HPLC purification. ¹H-NMR (MeOD): 8.98 d (J=0.9 Hz, 1H); 8.11 d (J=6.2 Hz, 1H); 7.96 m (2H); 7.81 dd (J=2.3 Hz/9.0 Hz, 1H); 7.72 d (J=2.1 Hz, 1H); 7.55 dd (J=0.9 Hz/6.0 Hz, 1H); 7.46 s (2H); 7.40 s (1H); 4.68 m (1H); 3.94 s (6H); 3.83 s (3H); 3.81 m (2H); 3.25 m (1H); 3.09 m (1H).

The following compounds were obtained in analogy to the preparation methods described above:

| | | Method | | |
|---|---|---|---|---|
| | Product; | analogous | ¹H-NMR (400 MHz) | |
| Ex. | reagents | to | δ [ppm] | Structure |
| 31 | 2-(7-Methoxy-benzofuran-2-yl)-6-trifluoromethoxy-quinoline-4-carboxylic acid[1-hydroxymethyl-2-(1H-pyrrolo[3,2-c]pyridin-3-yl)-ethyl]-amide;2-Amino-3-(1H-pyrrolo[3,2-c]pyridin-3-yl)-propan-1-oland2-(7-Methoxy-benzofuran-2-yl)-6-trifluoromethoxy-quinoline-4-carboxylic acid | 30 | ¹H-NMR (MeOD): 9.11 s(1H); 8.46 s (1H); 8.21 m(1H); 8.18 s (1H); 8.03 s(1H); 7.68 m (3H); 7.60 m(1H); 7.54 s (1H); 7.30 m(2H); 7.02 d (J = 7.5 Hz,1H); 4.67 m (1H); 4.06 s(3H); 3.84 m (2H); 3.29 m(1H); 3.14 m (1H). | 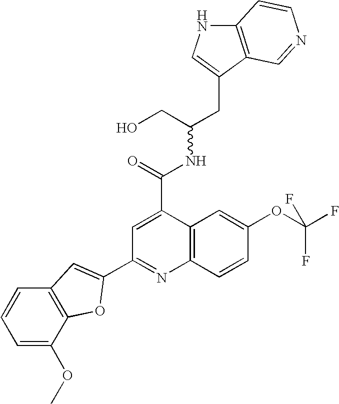 |

### EXAMPLE 32

### 5′-Fluoro-4-isopropoxy-biphenyl-3,3′-dicarboxylic acid 3-{[2-hydroxy-1-(1H-pyrrolo[3,2-c]pyridin-3-ylmethyl)-ethyl]-amide}3′-methylamide

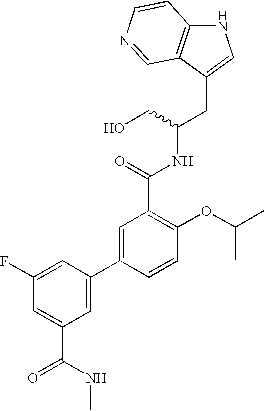

### 32a) 3′-Fluoro-4-isopropoxy-5′-methylcarbamoyl-biphenyl-3-carboxylic acid

The title compound was prepared according to the procedures described in example 1a-1c.

### 32b) 2-Amino-3-(1H-pyrrolo[3,2-c]pyridin-3-yl)-propan-1-ol

The title compound was prepared according to the procedure described in the example 27a-27e.

### 32c) 5′-Fluoro-4-isopropoxy-biphenyl-3,3′-dicarboxylic acid 3-{[2-hydroxy-1-(1H-pyrrolo[3,2-c]pyridin-3-ylmethyl)-ethyl]-amide}3′-methylamide

A solution of 3′-Fluoro-4-isopropoxy-5′-methylcarbamoyl-biphenyl-3-carboxylic acid (100 mg), 2-Amino-3-(1H-pyrrolo[3,2-c]pyridin-3-yl)-propan-1-ol dihydrochloride (63 mg), HOBt×water (51 mg) and EDCI (64 mg), triethylamine (0.057 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 2% yield after flash chromatography and HPLC purification. ¹H-NMR (MeOD): 9.20 s (1H); 8.51 s (1H); 8.26 m (2H); 7.94 m (1H); 7.86 dd (J=2.6 Hz/8.7 Hz, 1H); 7.70 d (J=6.2 Hz, 1H); 7.57 m (4H); 7.31 d (J=8.9 Hz, 1H); 4.50 m (1H); 3.75 m (2H); 3.31 m (2H); 2.98 s (3H); 1.40 m (6H).

The following compounds were obtained in analogy to the preparation methods described above:

| | | Method | | |
|---|---|---|---|---|
| | Product; | analogous | ¹H-NMR (400 MHz) | |
| Ex. | reagents | to | δ [ppm] | Structure |
| 33 | 3′-Chloro-4-isopropoxy-biphenyl-3,4′-dicarboxylicacid 3-{[2-hydroxy-1-(1H-pyrrolo[3,2-c]pyridin-3-ylmethyl)-ethyl]-amide}4′-methylamide;2-Amino-3-(1H-pyrrolo[3,2-c]pyridin-3-yl)-propan-1-oland3′-Chloro-4-isopropoxy-4′-methylcarbamoyl-biphenyl-3-carboxylic acid | 32 | ¹H-NMR (MeOD): 9.04 s(1H); 8.27 d (J = 2.5 Hz,1H); 8.17 d (J = 6.0 Hz,1H); 7.80 dd (J = 2.6 Hz/8.7 Hz, 1H); 7.74 d (J =1.5 Hz, 1H); 7.65 dd (J =1.7 Hz/7.9 Hz, 1H); 7.55d (J = 7.9 Hz, 1H); 7.52 d(J = 6.4 Hz, 1H); 7.40 s(1H); 7.27 d (J = 9.0 Hz,1H); 4.87 m (1H); 4.51 m(1H); 3.74 m (2H); 3.26 m(2H); 2.96 s (3H); 1.36 m(6H). | 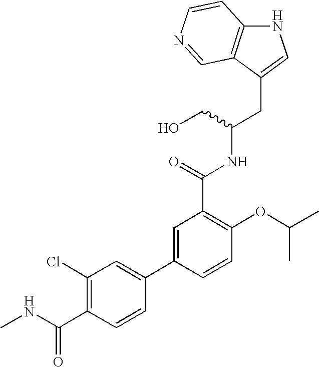 |

### EXAMPLE 34

### 7-(3,4-Dimethoxy-phenyl)-2,3,4,5-tetrahydro-benzo[b]oxepine-9-carboxylic acid [1-hydroxymethyl-2-(1H-pyrrolo[3,2-c]pyridin-3-yl)-ethyl]-amide

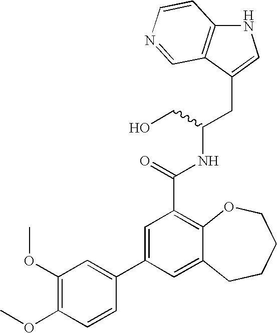

### 34a) 7-(3,4-Dimethoxy-phenyl)-2,3,4,5-tetrahydro-benzo[b]oxepine-9-carboxylic acid

The title compound was prepared according to the procedure described in example 26a-26h.

### 34b) 2-Amino-3-(1H-pyrrolo[3,2-c]pyridin-3-yl)-propan-1-ol

The title compound was prepared according to the procedure described in the example 27a-27e.

### 34c) 7-(3,4-Dimethoxy-phenyl)-2,3,4,5-tetrahydro-benzo[b]oxepine-9-carboxylic acid [1-hydroxymethyl-2-(1H-pyrrolo[3,2-c]pyridin-3-yl)-ethyl]-amide

A solution of 7-(3,4-Dimethoxy-phenyl)-2,3,4,5-tetrahydro-benzo[b]oxepine-9-carboxylic acid (100 mg), 2-Amino-3-(1H-pyrrolo[3,2-c]pyridin-3-yl)-propan-1-ol dihydrochloride (64 mg), HOBt×water (51 mg) and EDCI (64 mg), triethylamine (0.057 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 14% yield after flash chromatography. ¹H-NMR (MeOD): 9.14 s (1H); 8.21 d (J=6.2 Hz, 1H); 7.91 d (J=2.5 Hz, 1H); 7.65 d (J=6.2 Hz, 1H); 7.58 d (J=2.5 Hz, 1H); 7.52 s (1H); 7.18 m (2H); 7.04 d (J=8.3 Hz, 1H); 4.50 m (1H); 3.93 s (3H); 3.90 s (3H); 3.76 m (2H); 3.30 m (2H); 2.94 m (2H); 2.01 m (2H); 1.79 m (2H).

### EXAMPLE 35

### 4,3′-Dichloro-biphenyl-3,4′-dicarboxylic acid 4′-amide 3-{[2-hydroxy-1-(1H-pyrrolo[3,2-c]pyridin-3-ylmethyl)-ethyl]-amide}

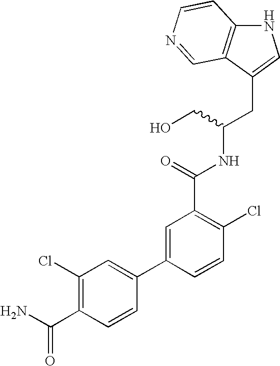

### 35a) 4′-Carbamoyl-4,3′-dichloro-biphenyl-3-carboxylic acid

The title compound was prepared in analogy to the procedure described in example 1b-1c. ESI-MS [M+1]: 311.

### 35b) 2-Amino-3-(1H-pyrrolo[3,2-c]pyridin-3-yl)-propan-1-ol

The title compound was prepared according to the procedure described in the example 27a-27e.

### 35c) 4,3′-Dichloro-biphenyl-3,4′-dicarboxylic acid 4′-amide 3-{[2-hydroxy-1-(1H-pyrrolo[3,2-c]pyridin-3-ylmethyl)-ethyl]-amide}

A solution of 4′-Carbamoyl-4,3′-dichloro-biphenyl-3-carboxylic acid (100 mg), 2-Amino-3-(1H-pyrrolo[3,2-c]pyridin-3-yl)-propan-1-ol dihydrochloride (68 mg), HOBt×water (54 mg) and EDCI (68 mg), triethylamine (0.061 ml) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 27% yield after flash chromatography and HPLC purification. ¹H-NMR (MeOD): 8.94 d (J=0.9 Hz, 1H); 8.14 d (J=5.8 Hz, 1H); 7.71 m (1H); 7.68 d (J=2.5 Hz, 1H); 7.63 s (1H); 7.58 d (J=1.7 Hz, 1H); 7.53 d (J=8.3 Hz, 1H); 7.42 m (2H); 7.34 s (1H); 4.52 m (1H); 3.76 m (2H); 3.26 m (1H); 3.11 m (1H).

### EXAMPLE 36

### N-[2-Hydroxy-1-(1H-pyrrolo[2,3-b]pyridin-3-ylmethyl)-ethyl]-5-(4-methylcarbamoyl-phenylethynyl)-2-trifluoromethoxy-benzamide

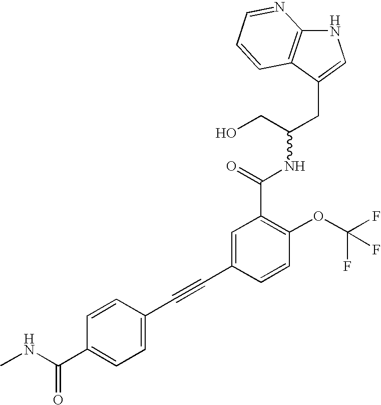

### 36a) 5-(4-Methylcarbamoyl-phenylethynyl)-2-trifluoromethoxy-benzoic acid

The title compound was prepared according to the procedure described in example 16a-16c.

### 36b) 2-Amino-3-(1H-pyrrolo[2,3-b]pyridin-3-yl)-propan-1-ol

The title compound was prepared according to the procedure described in example 1d-1e.

### 35c) N-[2-Hydroxy-1-(1H-pyrrolo[2,3-b]pyridin-3-ylmethyl)-ethyl]-5-(4-methylcarbamoyl-phenylethynyl)-2-trifluoromethoxy-benzamide

A solution of 5-(4-Methylcarbamoyl-phenylethynyl)-2-trifluoromethoxy-benzoic acid (100 mg), 2-Amino-3-(1H-pyrrolo[2,3-b]pyridin-3-yl)-propan-1-ol dihydrochloride (52 mg), HOBt×water (46 mg) and EDCI (48 mg), triethylamine (100 μl) in DMF (5 ml) was stirred overnight at ambient temperature. The reaction mixture was poured into water, extracted with ethyl acetate (3×). The combined organic layers were washed with brine and the solvent removed under vacuum. The title compound was obtained in 35% yield after flash chromatography. ¹H-NMR (DMSO-d₆): 10.78 s (1H); 8.52 m (1H); 8.35 d (J=8.3 Hz, 1H); 7.85 d (J=8.5 Hz, 2H); 7.65 m (4H); 7.46 m (1H); 7.29 d (J=7.9 Hz, 1H); 7.09 d (J=2.1 Hz, 1H); 7.03 m (2H); 4.77 m (1H); 4.14 m (1H); 3.45 m (2H); 2.96 m (1H); 2.83 m (1H); 2.77 d (J=4.5 Hz, 3H).

### EXAMPLE 37

### 4-Isopropoxy-3′,4′-dimethoxy-biphenyl-3-carboxylic acid [2-hydroxy-1-(1H-pyrrolo[2,3-c]pyridin-3-ylmethyl)-ethyl]-amide

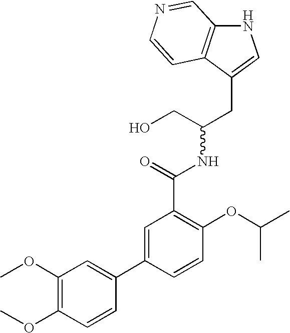

### 37a) Dimethyl-(1H-pyrrolo[2,3-c]pyridin-3-ylmethyl)-amine

To the mixture of dimethylamine hydrochloride (8.8 g) in isopropanol (400 mL) paraformaldehyde (3.3 g) was added. The mixture was stirred for 10 min and 4-azaindole (11.8 g) was added. This mixture was heated for 1.5 h at 80° C. After cooling the mixture was filtered and concentrated under reduced pressure. The residue was diluted with water (100 ml) and HCl (10 mL), washed with ethyl ether and concentrated under reduced pressure. The residue was dissolved in 30% NaOH and extracted with ethyl ether. Organic phases were collected, dried (K₂CO₃) and concentrated under reduced pressure to give the title compound (10 g).

### 37b) 2-Acetylamino-2-(1H-pyrrolo[2,3-c]pyridin-3-ylmethyl)-malonic acid diethyl ester

The mixture of Dimethyl-(1H-pyrrolo[2,3-c]pyridin-3-ylmethyl)-amine (3.5 g), diethyl acetamidomalonate (4.34 g), NaOH (250 mg), xylenes (35 mL) was refluxed for 5 h under Ar. The hot mixture was filtered and cooled. The precipitate formed was filtered, washed with hexane and dried to give the title compound (3.5 g).

### 37c) 2-Acetylamino-3-(1H-pyrrolo[2,3-c]pyridin-3-yl)-propionic acid ethyl ester

To the solution of 2-Acetylamino-2-(1H-pyrrolo[2,3-c]pyridin-3-ylmethyl)-malonic acid diethyl ester (3.5 g) in ethanol (40 mL) the solution of KOH (0.72 g) in water (4 mL) was added. The mixture was stirred at rt for 3 h, acidified with HCl (1 mL) and concentrated at 30° C. under reduced pressure. Water (10 mL) was added and the mixture was acidified to pH 1 and concentrated under reduced pressure. The residue was heated at 130° C. under Ar for 1 h. After cooling the residue was dissolved in water (minimum) and basified with saturated K₂CO₃. The solid formed was filtered, washed with ethyl ether and dried to give the title compound (2.5 g).

### 37d) N-[1-Hydroxymethyl-2-(1H-pyrrolo[2,3-c]pyridin-3-yl)-ethyl]-acetamide

The suspension of 2-Acetylamino-3-(1H-pyrrolo[2,3-c]pyridin-3-yl)-propionic acid ethyl ester (2.75 g), LiBH₄ (0.9 g), ethyl ether (20 mL) was stirred for 24 h at rt. The reaction was quenched at −20° C. with cooled MeOH and stirred for 3 h. Reaction mixture was acidified with KHSO₄ to pH 3 and concentrated under reduced pressure. The residue was dissolved with saturated K₂CO₃ and extracted with acetonitrile. Organic phase was dried and concentrated to give the title compound (1.2 g).

### 37e) 2-Amino-3-(1H-pyrrolo[2,3-c]pyridin-3-yl)-propan-1-ol

N-[1-Hydroxymethyl-2-(1H-pyrrolo[2,3-c]pyridin-3-yl)-ethyl]-acetamide (1 g) in 20% HCl (1 mL) was stirred overnight at rt. The resulting mixture was heated at 60° C. for 4 h. After concentration the residue was crystallized from methanol/chloroform (1:4) yielding the title compound (1.25 g) as dihydrochloride salt. ESI-MS [M+1]: 192.

### 37f) 4-Isopropoxy-3′,4′-dimethoxy-biphenyl-3-carboxylic acid

The title compound was prepared in analogy to the procedure described in example 1a-1c. ESI-MS [M+1]: 317.

### 37g) 4-Isopropoxy-3′,4′-dimethoxy-biphenyl-3-carboxylic acid [2-hydroxy-1-(1H-pyrrolo[2,3-c]pyridin-3-ylmethyl)-ethyl]-amide

A solution of 4-Isopropoxy-3′,4′-dimethoxy-biphenyl-3-carboxylic acid (250 mg), 2-Amino-3-(1H-pyrrolo[2,3-c]pyridin-3-yl)-propan-1-ol dihydrochloride (302 mg), HOBt×water (242 mg) and EDCI (303 mg), triethylamine (0.29 ml) in DMF (10 ml) was stirred overnight at ambient temperature. The reaction mixture was poured into water, extracted with ethyl acetate (3×). The combined organic layers were washed with brine and the solvent removed under vacuum. The title compound was obtained in 43% yield after flash chromatography and HPLC purification. ¹H-NMR (DMSO-d₆): 11.40 s (1H); 8.68 s (1H); 8.42 d (J=8.3 Hz, 1H); 8.08 m (2H); 7.67 m (2H); 7.41 s (1H); 7.18 d (J=8.9 Hz, 1H); 7.12 m (2H); 6.99 d (J=8.5 Hz, 1H); 4.97 s (1H); 4.75 m (1H); 4.21 m (1H); 3.81 s (3H); 3.75 s (3H); 3.44 m (2H); 2.98 m (2H); 1.21 m (6H).

### EXAMPLE 38

### 4,3′-Dichloro-biphenyl-3,4′-dicarboxylic acid 4′-amide 3-{[2-hydroxy-1-(1H-pyrrolo[2,3-c]pyridin-3-ylmethyl)-ethyl]-amide}

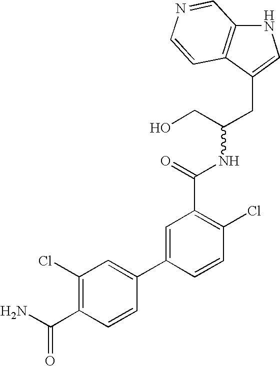

### 38a) 4′-Carbamoyl-4,3′-dichloro-biphenyl-3-carboxylic acid

The title compound was prepared in analogy to the procedure described in example 1b-1c. ESI-MS [M+1]: 311.

### 38b) 2-Amino-3-(1H-pyrrolo[2,3-c]pyridin-3-yl)-propan-1-ol

The title compound was prepared according to the procedure described in example 37a-37e.

### 38c) 4,3′-Dichloro-biphenyl-3,4′-dicarboxylic acid 4′-amide 3-{[2-hydroxy-1-(1H-pyrrolo[2,3-c]pyridin-3-ylmethyl)-ethyl]-amide}

A solution of 4′-Carbamoyl-4,3′-dichloro-biphenyl-3-carboxylic acid (150 mg), 2-Amino-3-(1H-pyrrolo[2,3-c]pyridin-3-yl)-propan-1-ol dihydrochloride (165 mg), HOBt×water (133 mg) and EDCI (165 mg), triethylamine (0.16 ml) in DMF (10 ml) was stirred overnight at ambient temperature. The reaction mixture was poured into water, extracted with ethyl acetate (3×). The combined organic layers were washed with brine and the solvent removed under vacuum. The title compound was obtained in 1% yield after flash chromatography and HPLC purification. ¹H-NMR (MeOD): 8.81 s (1H); 8.07 m (1H); 7.98 m (1H); 7.81 s (1H); 7.67-7.43 m (6H); 4.47 m (1H); 3.73 m (2H); 3.22 m (1H); 3.04 m (1H).

### EXAMPLE 39

### 3′-Chloro-4-isopropoxy-biphenyl-3,4′-dicarboxylic acid 3-{[2-hydroxy-1-(1H-pyrrolo[2,3-c]pyridin-3-ylmethyl)-ethyl]-amide}4′-methylamide

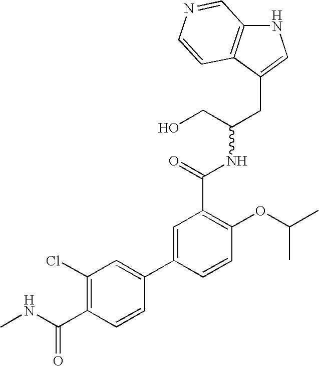

### 39a) 3′-Chloro-4-isopropoxy-4′-methylcarbamoyl-biphenyl-3-carboxylic acid

The title compound was prepared in analogy to the procedures described in examples 1a-1c. ESI-MS [M+1]: 348.

### 39b) 2-Amino-3-(1H-pyrrolo[2,3-c]pyridin-3-yl)-propan-1-ol

The title compound was prepared according to the procedure described in example 37a-37e.

### 39c) 3′-Chloro-4-isopropoxy-biphenyl-3,4′-dicarboxylic acid 3-{[2-hydroxy-1-(1H-pyrrolo[2,3-c]pyridin-3-ylmethyl)-ethyl]-amide}4′-methylamide

A solution of 3′-Chloro-4-isopropoxy-4′-methylcarbamoyl-biphenyl-3-carboxylic acid (150 mg), 2-Amino-3-(1H-pyrrolo[2,3-c]pyridin-3-yl)-propan-1-ol dihydrochloride (165 mg), HOBt×water (133 mg) and EDCI (165 mg), triethylamine (0.16 ml) in DMF (10 ml) was stirred overnight at ambient temperature. The reaction mixture was poured into water, extracted with ethyl acetate (3×). The combined organic layers were washed with brine and the solvent removed under vacuum. The title compound was obtained in 17% yield after flash chromatography and HPLC purification. ¹H-NMR (DMSO-d₆): 11.31 s (1H); 8.65s (1H); 8.39 m (2H); 8.20s (1H); 8.14 d (J=2.5 Hz, 1H); 8.03 d (J=5.3 Hz, 1H); 7.79 d (J=8.9 Hz/2.6 Hz, 1H); 7.71 d (J=1.7 Hz, 1H); 7.62 m (2H); 7.46 d (J=7.9 Hz, 1H); 7.37 s (1H); 7.25 d (J=9.0 Hz, 1H); 4.79 m (1H); 4.21 m (1H); 3.44 m (2H); 2.97 m (2H); 2.73 d (J=4.7 Hz, 3H); 1.21 m (6H).

### EXAMPLE 40

### 5′-Fluoro-4-isopropoxy-biphenyl-3,3′-dicarboxylic acid 3-{[2-hydroxy-1-(1H-pyrrolo[2,3-c]pyridin-3-ylmethyl)-ethyl]-amide}3′-methylamide

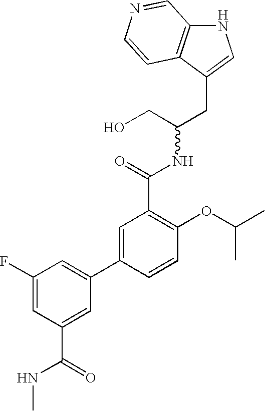

### 40a) 3′-Fluoro-4-isopropoxy-5′-methylcarbamoyl-biphenyl-3-carboxylic acid

The title compound was prepared as described in example 19b-19f.

### 40b) 2-Amino-3-(1H-pyrrolo[2,3-c]pyridin-3-yl)-propan-1-ol

The title compound was prepared according to the procedure described in example 37a-37e.

### 40c) 5′-Fluoro-4-isopropoxy-biphenyl-3,3′-dicarboxylic acid 3-{[2-hydroxy-1-(1H-pyrrolo[2,3-c]pyridin-3-ylmethyl)-ethyl]-amide}3′-methylamide

A solution of 3′-Fluoro-4-isopropoxy-5′-methylcarbamoyl-biphenyl-3-carboxylic acid (250 mg), 2-Amino-3-(1H-pyrrolo[2,3-c]pyridin-3-yl)-propan-1-ol dihydrochloride (289 mg), HOBt×water (132 mg) and EDCI (289 mg), triethylamine (0.27 ml) in DMF (10 ml) was stirred overnight at ambient temperature. The reaction mixture was poured into water, extracted with ethyl acetate (3×). The combined organic layers were washed with brine and the solvent removed under vacuum. The title compound was obtained in 44% yield after flash chromatography and HPLC purification. ¹H-NMR (DMSO-d₆): 11.40 s (1H); 8.68 m (2H); 8.40 d (J=8.1 Hz, 1H); 8.23 d (J=2.5 Hz, 1H); 8.12 m (1H); 7.94 s (1H); 7.81 dd (J=8.7 Hz/2.6 Hz, 1H); 7.61 d (J=9.6 Hz, 1H); 7.53 d (J=9.6 Hz, 1H); 7.43 s (1H); 7.25 d (J=9.6 Hz, 1H); 4.80 m (1H); 4.22 m (1H); 3.45 m (2H); 2.98 m (2H); 2.79 d (J=4.5 Hz, 3H); 1.22 m (6H).

### EXAMPLE 41

### 6-Bromo-2-(3,4,5-trimethoxy-phenyl)-quinoline-4-carboxylic acid [2-hydroxy-1-(1H-pyrrolo[2,3-c]pyridin-3-ylmethyl)-ethyl]-amide

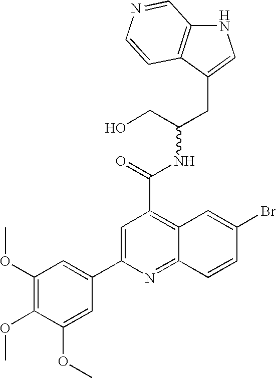

### 41a) 6-Bromo-2-(3,4,5-trimethoxy-phenyl)-quinoline-4-carboxylic acid

The title compound was prepared in analogy to the procedure described in example 6a. ESI-MS [M+1]: 419.

### 41b) 2-Amino-3-(1H-pyrrolo[2,3-c]pyridin-3-yl)-propan-1-ol

The title compound was prepared according to the procedure described in example 37a-37e.

### 41c) 6-Bromo-2-(3,4,5-trimethoxy-phenyl)-quinoline-4-carboxylic acid [2-hydroxy-1-(1H-pyrrolo[2,3-c]pyridin-3-ylmethyl)-ethyl]-amide

A solution of 6-Bromo-2-(3,4,5-trimethoxy-phenyl)-quinoline-4-carboxylic acid (250 mg), 2-Amino-3-(1H-pyrrolo[2,3-c]pyridin-3-yl)-propan-1-ol dihydrochloride (229 mg), HOBt×water (184 mg) and EDCI (229 mg), triethylamine (0.22 ml) in DMF (10 ml) was stirred overnight at ambient temperature. The reaction mixture was poured into water, extracted with ethyl acetate (3×). The combined organic layers were washed with brine and the solvent removed under vacuum. The title compound was obtained in 48% yield after flash chromatography and HPLC purification. ¹H-NMR (DMSO-d₆): 11.38 s (1H); 8.73 d (J=8.5 Hz, 1H); 8.69 m (1H); 8.04 d (J=2.1 Hz, 1H); 8.02 d (J=3.6 Hz, 2H); 7.99 d (J=3.2 Hz, 1H); 7.87 dd (J=2.3 Hz/9.0 Hz, 1H); 7.62 dd (J=9.0 Hz/2.3 Hz, 1H); 7.63 d (J=5.5 Hz/0.9 Hz, 1H); 7.51 s (2H); 7.45 s (1H); 4.35 m (1H); 3.90 s (6H); 3.75 s (3H); 3.58 m (2H); 3.05 m (1H); 2.95 m (1H).

### EXAMPLE 42

### N-[2-Hydroxy-1-(1H-pyrrolo[2,3-c]pyridin-3-ylmethyl)-ethyl]-2-isopropoxy-5-(2-methoxy-phenylethynyl)-benzamide

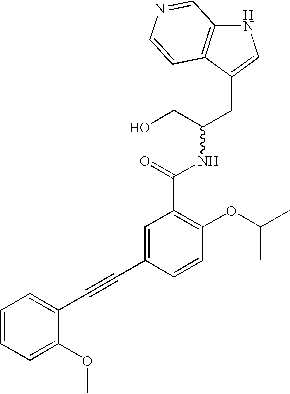

### 42a) 2-Isopropoxy-5-(2-methoxy-phenylethynyl)-benzoic acid

The title compound was prepared according to the procedures described in example 3a-3c.

### 42b) 2-Amino-3-(1H-pyrrolo[2,3-c]pyridin-3-yl)-propan-1-ol

The title compound was prepared according to the procedure described in example 37a-37e.

### 42c) N-[2-Hydroxy-1-(1H-pyrrolo[2,3-c]pyridin-3-ylmethyl)-ethyl]-2-isopropoxy-5-(2-methoxy-phenylethynyl)-benzamide

A solution of 2-Isopropoxy-5-(2-methoxy-phenylethynyl)-benzoic acid (250 mg), 2-Amino-3-(1H-pyrrolo[2,3-c]pyridin-3-yl)-propan-1-ol dihydrochloride (308 mg), HOBt×water (247 mg) and EDCI (309 mg), triethylamine (0.29 ml) in DMF (10 ml) was stirred overnight at ambient temperature. The reaction mixture was poured into water, extracted with ethyl acetate (3×). The combined organic layers were washed with brine and the solvent removed under vacuum. The title compound was obtained in 56% yield after flash chromatography and HPLC purification. ¹H-NMR (DMSO-d₆): 11.51 s (1H); 8.71 s (1H); 8.33 d (J=8.3 Hz, 1H); 8.06 d (J=5.7 Hz, 1H); 7.92 d (J=2.3 Hz, 1H); 7.69 dd (J=5.7 Hz/0.8 Hz, 1H); 7.53 dd (J=2.3 Hz/8.5 Hz, 1H); 7.47 s (1H); 7.44 dd (J=1.7 Hz/7.5 Hz, 1H); 7.35 m (1H); 7.16 d (J=8.9 Hz, 1H); 7.05 d (J=7.9 Hz, 1H); 6.94 m (1H); 4.98 s (1H); 4.76 m (1H); 4.20 m (1H); 3.83 s (3H); 3.44 m (2H); 2.98 m (2H); 1.20 m (6H).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07 075 645.7, filed Jul. 26, 2007, and U.S. Provisional Application Ser. No. 60/952,037, filed Jul. 26, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the formula I 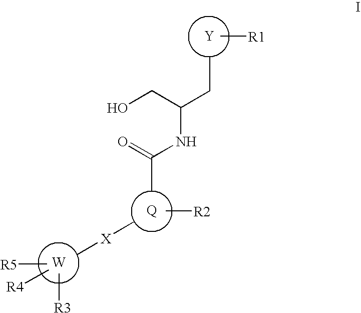
in which
R1 is hydrogen, fluorine, C₁-C₆-alkyl in which the alkyl chain may optionally be substituted one or more times by fluorine;
R2 is hydrogen, halogen, nitro, amino, cyano, C₁-C₆-alkyl, C₂-C₆-alkenyl or C₂-C₆-alkynyl, C₃-C₇-cycloalkyl, hydroxy-C₁-C₆-alkylene, hydroxy-C₃-C₆-alkenylene, hydroxy-C₃-C₆-alkynylene, C₁-C₆-alkyloxy, C₁-C₆-alkyloxy-C₁-C₆-alkylene, C₃-C₇-cycloalkyloxy, C₃-C₇-cycloalkyl-C₁-C₆-alkylenoxy, C₃-C₇-cycloalkyloxy-C₁-C₆-alkylene, C₁-C₆-alkyloxy-C₃-C₆-alkenylene, C₁-C₆-alkyloxy-C₃-C₆-alkynylene, C₁-C₆-alkylamino-C₁-C₆-alkylene, di(C₁-C₆-alkyl)amino-C₁-C₆-alkylene; where the hydrocarbon chains therein may optionally be substituted one or more times by fluorine, cyano, hydroxy, amino or by the groups: 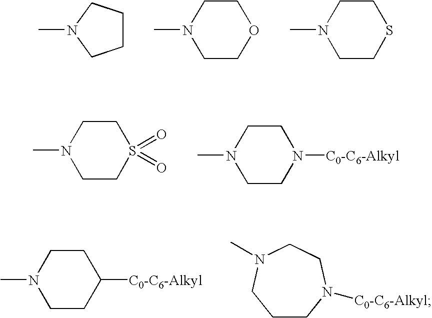
R3, R4, R5 are independently of one another hydrogen, hydroxy, halogen, nitro, amino, cyano, phenyl, C₁-C₆-alkyl, C₂-C₆-alkenyl or C₂-C₆-alkynyl, C₃-C₇-cycloalkyl, C₃-C₇-cycloalkyl-C₁-C₆-alkylene, C₃-C₇-heterocycloalkyl,
where the hydrocarbon chains therein may optionally be substituted one or more times by fluorine, cyano or by the radicals: 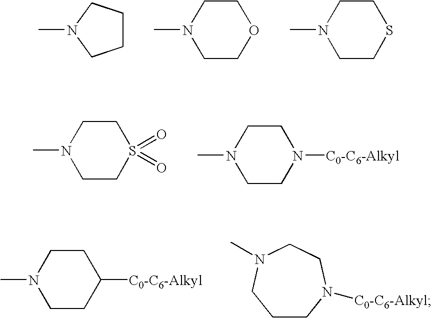
or
independently of one another hydroxy-C₁-C₆-alkylene, hydroxy-C₃-C₆-alkenylene, hydroxy-C₃-C₆-alkynylene, C₁-C₆-alkyloxy, C₃-C₇-cycloalkyloxy, C₃-C₇-cycloalkyl-C₁-C₆-alkylenoxy, C₁-C₆-alkyloxy-C₁-C₆-alkylene, C3-C7-cycloalkyloxy-C₁-C₆-alkylene, C₁-C₆-alkyloxy-C₃-C₆-alkenylene, C₁-C₆-alkyloxy-C₃-C₆-alkynylene, C₁-C₆-alkyloxyphenyl-C₁-C₆-alkylene, phenyloxy-C₁-C₆-alkylene,
C₁-C₆-alkylamino, di(C₁-C₆-alkyl)amino, C₁-C₆-alkylamino-C₁-C₆-alkylene, di(C₁-C₆)-alkylamino-C₁-C₆-alkylene, C₃-C₇-cycloalkyl-(C₀-C₆-alkyl)amino, C₁-C₆-acyl-(C₀-C₆-alkyl)amido, C₁-C₆-alkylaminocarbonyl, di(C₁-C₆alkyl)aminocarbonyl, (C₃-C₇-cycloalkyl)aminocarbonyl, di(C₃-C₇-cycloalkyl)aminocarbonyl, C₃-C₇-cycloalkyl-C₁-C₆-alkyleneaminocarbonyl, C₁-C₆-alkylcarbonyl, C₃-C₇-cycloalkylcarbonyl,
carboxy, carboxamido [—C(O)NH₂], C₁-C₆-alkyloxycarbonyl, C₁-C₃-alkylsulphanyl, C₁-C₆-alkysulphonyl, C₃-C₇-cycloalkylsulphonyl, C₃-C₇-cycloalkyl-C₁-C₆-alkylenesulphonyl,
C₁-C₆-alkylaminosulphonyl, di(C₁-C₆-alkyl)aminosulphonyl, (C₃₋₇-cycloalkyl)aminosulphonyl, di(C₃-C₇-cycloalkyl)aminosulphonyl, C₃-C₇-cycloalkyl-C₁-C₆-alkyleneaminosulphonyl, C₁-C₆-alkylsulphonylamido, —N(C₀-C₆-alkyl)-C(O)—C₁-C₆-alkyl, —N(C₀-C₆-alkyl)-C(O)—C₃-C₇-cycloalkyl, —N(C₀-C₆-alkyl)-C(O)—N-di(C₀-C₆-alkyl), —N(C₀-C₆-alkyl)-C(O)—O—(C₀-C₆)alkyl, —N(C₀-C₆-alkyl)-C(O)—NH—C₃-C₇-cycloalkyl,
—N(C₀-C₆-alkyl)-SO₂—C₁-C₆-alkyl, —N(C₀-C₆-alkyl)-SO₂—C₃-C₇-cycloalkyl, —N(C₀-C₆-alkyl)-SO₂—N-di(C₀-C₆-alkyl), —N(C₀-C₆-alkyl)-SO₂—NH—(C₃-C₇)-cycloalkyl,
—C(O)—N(H)—C₂-C₆-alkylene-(C₁-C₆-alkyl)amine, —C(O)—N(H)—C₂-C₆-alkylene-[di(C₁-C₆-alkyl)]amine, —C(O)—N(H)—C₂-C₆-alkylene-(C₃-C₇-cycloalkyl)amine, —C(O)—N(H)—C₂-C₆-alkylene-(C₃-C₇-cycloalkyl-C₁-C₆-alkyl)amine, —S(O₂)—N(H)—C₂-C₆-alkylene-(C₁-C₆-alkyl)amine, —S(O₂)—N(H)—C₂-C₆-alkylene-[di(C₁-C₆-alkyl)]amine, —S(O₂)—N(H)—C₂-C₆-alkylene-(C₃-C₇-cycloalkyl)amine, —S(O₂)—N(H)—C₂-C₆-alkylene-(C₃-C₇-cycloalkyl-C₁-C₆-alkylene)amine, —O—C₂-C₆-alkylene-(C₁-C₆-alkyl)amine, —O—C₂-C₆-alkylene-[di(C₁-C₆-alkylene)]amine,
or the radicals: 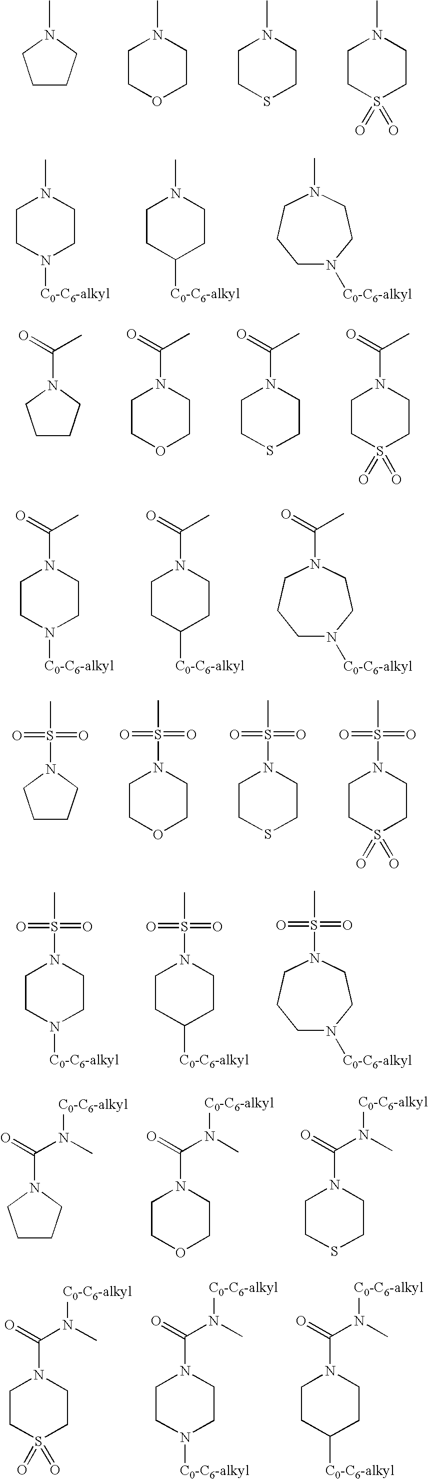 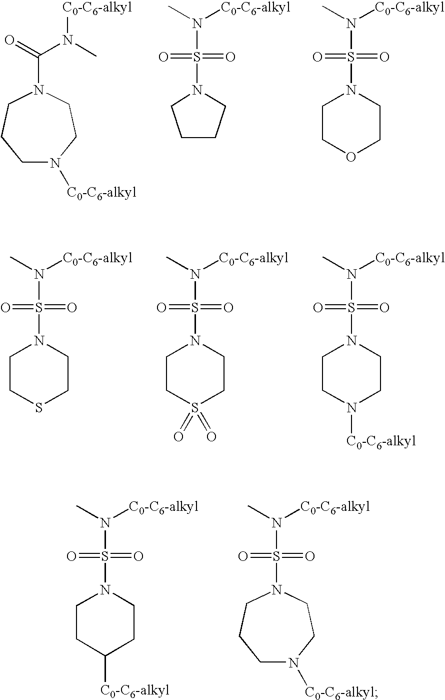
R3 and R4 may together form heterocycloalkyl, cycloalkyl;
Q is an aryl or heteroaryl group
or the group 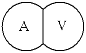
in which
A is a monocyclic aryl or a monocyclic heteroaryl group;
V is a cycloalkylen, cycloalkenylen, heterocycloalkylen or heterocycloalkenylen group;
X is a bond or an ethynyl group;
W is an aryl or heteroaryl group;
Y is selected from the groups 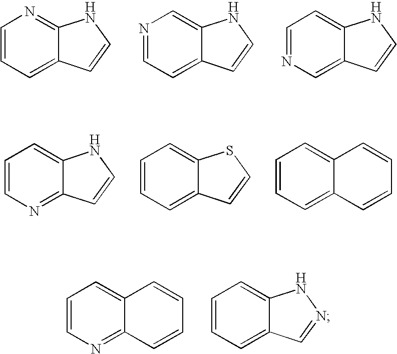
where
R1 substitutes one or more positions of the aryl ring in the radical Y;
substitutes one or more positions of the aryl or heteroaryl ring in the radical Q or in the radical V.

2. Compounds according to claim 1, characterized in that
Y is selected from the heteroaryl groups:

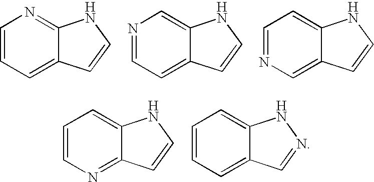

3. Compounds according to claim 2, namely arylmethylene substituted N-acyl-β-amino alcohols of the formula II 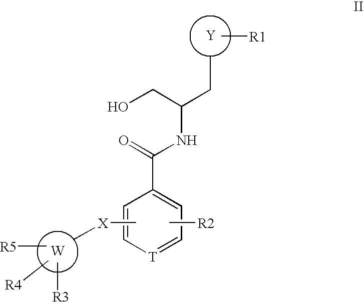
in which
T is a nitrogen atom or a CH group;
and
R1, R2, R3, R4, R5, X and W have the same meaning as defined.

4. Compounds according to claim 2, namely arylmethylene substituted N-acyl-β-amino alcohols of the formula III 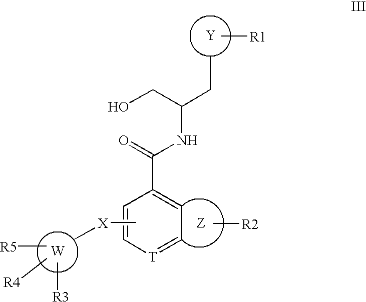
in which
T is a nitrogen atom or a CH group;
Z is a monocyclic aryl or a monocyclic heteroaryl group or a cycloalkylen, cycloalkenylen, heterocycloalkylen or heterocycloalkenylen group;
and
R1, R2, R3, R4, R5, X and W have the same meaning as defined.

5. Compounds according to claim 2, namely arylmethylene substituted N-acyl-β-amino alcohols of the formula IV 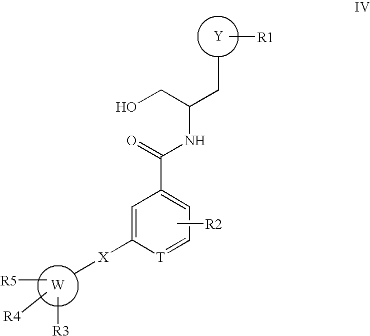
in which
T is a nitrogen atom or a CH group;
and
R1, R2, R3, R4, R5, X, Y and W have the same meaning as defined.

6. Compounds according to claim 2, namely arylmethylene substituted N-acyl-β-amino alcohols of the formula V 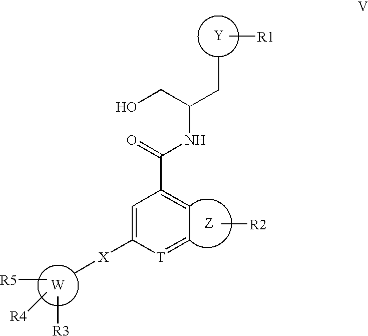
in which
T is a nitrogen atom or a CH group;
Z is a monocyclic aryl or a monocyclic heteroaryl group or a cycloalkylen, cycloalkenylen, heterocycloalkylen or heterocycloalkenylen group;
and
R1, R2, R3, R4, R5, X, Y and W have the same meaning as defined.

7. Compounds according to claim 1, namely
1 5′-Fluoro-4-isopropoxy-biphenyl-3,3′-dicarboxylic acid 3-{[2-hydroxy-1-(1H-pyrrolo[2,3-b]pyridin-3-ylmethyl)-ethyl]-amide}3′-methylamide;
2 3′-Chloro-4-isopropoxy-biphenyl-3,4′-dicarboxylic acid 3-{[2-hydroxy-1-(1H-pyrrolo[2,3-b]pyridin-3-ylmethyl)-ethyl]-amide}-4′-methylamide;
3 N-[1-Hydroxymethyl-2-(1H-pyrrolo[2,3-b]pyridin-3-yl)-ethyl]-2-isopropoxy-5-(2-methoxy-phenylethynyl)-benzamide;
4 N-[1-Hydroxymethyl-2-(1H-pyrrolo[2,3-b]pyridin-3-yl)-ethyl]-2-isopropoxy-5-phenylethynyl-benzamide;
5 6-(3-Chloro-4-methylcarbamoyl-phenyl)-2,2-dimethyl-2H-chromene-8-carboxylic acid [2-hydroxy-1-(1H-pyrrolo[2,3-b]pyridin-3-ylmethyl)-ethyl]-amide;
6 6-Methoxy-2-(3,4,5-trimethoxy-phenyl)-quinoline-4-carboxylic acid ((R)-2-benzo[b]-thiophen-3-yl-1-hydroxy-methyl-ethyl)-amide;
7 4-Ethoxy-3′-methoxy-biphenyl-3-carboxylic acid ((R)-2-benzo[b]thiophen-3-yl-1-hydroxymethyl-ethyl)-amide;
8 6-Methoxy-2-(3,4,5-trimethoxy-phenyl)-quinoline-4-carboxylic acid (1-hydroxymethyl-2-quinolin-3-yl-ethyl)-amide;
9 6-Methoxy-2-(3,4,5-trimethoxy-phenyl)-quinoline-4-carboxylic acid ((R)-2-hydroxy-1-naphthalen-2-ylmethyl-ethyl)-amide;
10 6-Iodo-2-(3,4,5-trimethoxy-phenyl)-quinoline-4-carboxylic acid ((R)-2-hydroxy-1-naphthalen-2-ylmethyl-ethyl)-amide;
11 6-Methoxy-2-(3,4,5-trimethoxy-phenyl)-quinoline-4-carboxylic acid (2-hydroxy-1-naphthalen-1-ylmethyl-ethyl)-amide;
12 6-Methoxy-2-(3,4,5-trimethoxy-phenyl)-quinoline-4-carboxylic acid (2-hydroxy-1-naphthalen-2-ylmethyl-ethyl)-amide.

8. Compounds according to claim 1, namely
13 3′-Chloro-4-trifluoromethoxy-biphenyl-3,4′-dicarboxylic acid 3-{[2-hydroxy-1-(4-fluoro-1H-indazol-3-ylmethyl)-ethyl]-amide}-4′-methylamide;
14 3′-Chloro-4-trifluoromethoxy-biphenyl-3,4′-dicarboxylic acid 3-{[2-hydroxy-1-(7-fluoro-1H-indazol-3-ylmethyl)-ethyl]-amide}-4′-methylamide;
15 3′-Chloro-4-trifluoromethoxy-biphenyl-3,4′-dicarboxylic acid 3-{[2-hydroxy-1-(7-trifluoromethyl-1H-indazol-3-ylmethyl)-ethyl]-amide}-4′-methylamide;
16 N-[2-hydroxy-1-(4-fluoro-1H-indazol-3-ylmethyl)-ethyl]-2-trifluoromethoxy-5-[(4-methylaminocarbonyl-phenyl)-ethynyl]-benzamide;
17 N-[2-hydroxy-1-(7-fluoro-1H-indazol-3-ylmethyl)-ethyl]-2-trifluoromethoxy-5-[(4-methylaminocarbonyl-phenyl)-ethynyl]-benzamide;
18 N-[2-hydroxy-1-(7-trifluoromethyl-1H-indazol-3-ylmethyl)-ethyl]-2-trifluoromethoxy-5-[(4-methylaminocarbonyl-phenyl)-ethynyl]-benzamide;
19 6-Bromo-2-(3,4,5-trimethoxy-phenyl)-quinoline-4-carboxylic acid [2-hydroxy-1-(1H-pyrrolo[3,2-b]pyridin-3-ylmethyl)-ethyl]-amide;
20 2-(7-Methoxy-benzofuran-2-yl)-6-trifluoromethoxy-quinoline-4-carboxylic acid [2-hydroxy-1-(1H-pyrrolo[3,2-b]pyridin-3-ylmethyl)-ethyl]-amide;
21 2-(3-Fluoro-4-methoxy-phenyl)-6-methoxy-quinazoline-4-carboxylic acid [2-hydroxy-1-(1H-pyrrolo[3,2-b]pyridin-3-ylmethyl)-ethyl]-amide;
22 N-[1-Hydroxymethyl-2-(1H-pyrrolo[3,2-b]pyridin-3-yl)-ethyl]-2-isopropoxy-5-(2-methoxy-phenylethynyl)-benzamide;
23 N-[1-Hydroxymethyl-2-(1H-pyrrolo[3,2-b]pyridin-3-yl)-ethyl]-2-isopropoxy-5-phenylethynyl-benzamide;
24 5′-Fluoro-4-isopropoxy-biphenyl-3,3′-dicarboxylic acid 3-{[1-hydroxymethyl-2-(1H-pyrrolo[3,2-b]pyridin-2-yl)-ethyl]-amide}3′-methylamide;
25 3′-Chloro-4-isopropoxy-biphenyl-3,4′-dicarboxylic acid 3-{[1-hydroxymethyl-2-(1H-pyrrolo[3,2-b]pyridin-3-yl)-ethyl]-amide}-4′-methylamide;
26 7-(3,4-Dimethoxy-phenyl)-2,3,4,5-tetrahydro-benzo[b]oxepine-9-carboxylic acid [1-hydroxymethyl-2-(1H-pyrrolo[3,2-b]pyridin-3-yl)-ethyl]-amide
27 N-[1-Hydroxymethyl-2-(1H-pyrrolo[3,2-c]pyridin-3-yl)-ethyl]-2-isopropoxy-5-(2-methoxy-phenylethynyl)-benzamide
28 N-[1-Hydroxymethyl-2-(1H-pyrrolo[3,2-c]pyridin-3-yl)-ethyl]-2-isopropoxy-5-phenylethynyl-benzamide;
29 2-(3-Fluoro-4-methoxy-phenyl)-6-methoxy-quinazoline-4-carboxylic acid [2-hydroxy-1-(1H-pyrrolo[3,2-c]pyridin-3-ylmethyl)-ethyl]-amide;
30 6-Bromo-2-(3,4,5-trimethoxy-phenyl)-quinoline-4-carboxylic acid [2-hydroxy-1-(1H-pyrrolo[3,2-c]pyridin-3-ylmethyl)-ethyl]-amide;
31 2-(7-Methoxy-benzofuran-2-yl)-6-trifluoromethoxy-quinoline-4-carboxylic acid [1-hydroxymethyl-2-(1H-pyrrolo[3,2-c]pyridin-3-yl)-ethyl]-amide;
32 5′-Fluoro-4-isopropoxy-biphenyl-3,3′-dicarboxylic acid 3-{[2-hydroxy-1-(1H-pyrrolo[3,2-c]pyridin-3-ylmethyl)-ethyl]-amide}3′-methylamide;
33 3′-Chloro-4-isopropoxy-biphenyl-3,4′-dicarboxylic acid 3-{[2-hydroxy-1-(1H-pyrrolo[3,2-c]pyridin-3-ylmethyl)-ethyl]-amide}-4′-methylamide;
34 7-(3,4-Dimethoxy-phenyl)-2,3,4,5-tetrahydro-benzo[b]oxepine-9-carboxylic acid [1-hydroxymethyl-2-(1H-pyrrolo[3,2-c]pyridin-3-yl)-ethyl]-amide;
35 4,3′-Dichloro-biphenyl-3,4′-dicarboxylic acid 4′-amide 3-{[2-hydroxy-1-(1H-pyrrolo[3,2-c]pyridin-3-ylmethyl)-ethyl]-amide};
36 N-[2-Hydroxy-1-(1H-pyrrolo[2,3-b]pyridin-3-ylmethyl)-ethyl]-5-(4-methylcarbamoyl-phenylethynyl)-2-trifluoromethoxy-benzamide;
37 4-Isopropoxy-3′,4′-dimethoxy-biphenyl-3-carboxylic acid [2-hydroxy-1-(1H-pyrrolo[2,3-c]pyridin-3-ylmethyl)-ethyl]-amide;
38 4,3′-Dichloro-biphenyl-3,4′-dicarboxylic acid 4′-amide 3-{[2-hydroxy-1-(1H-pyrrolo[2,3-c]pyridin-3-ylmethyl)-ethyl]-amide};
39 3′-Chloro-4-isopropoxy-biphenyl-3,4′-dicarboxylic acid 3-{[2-hydroxy-1-(1H-pyrrolo[2,3-c]pyridin-3-ylmethyl)-ethyl]-amide}4′-methylamide;
40 5′-Fluoro-4-isopropoxy-biphenyl-3,3′-dicarboxylic acid 3-{[2-hydroxy-1-(1H-pyrrolo[2,3-c]pyridin-3-ylmethyl)-ethyl]-amide}3′-methylamide;
41 6-Bromo-2-(3,4,5-trimethoxy-phenyl)-quinoline-4-carboxylic acid [2-hydroxy-1-(1H-pyrrolo[2,3-c]pyridin-3-ylmethyl)-ethyl]-amide;
42 N-[2-Hydroxy-1-(1H-pyrrolo[2,3-c]pyridin-3-ylmethyl)-ethyl]-2-isopropoxy-5-(2-methoxy-phenylethynyl)-benzamide.

9. Process for preparing compounds of the formula I of claim 1, wherein an amino alcohol of the formula VI 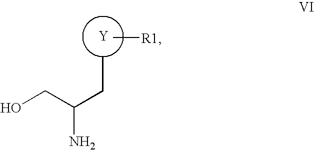
in which the radical R1 has the same meaning as defined in claim 1,
is coupled with a carboxylic acid of the formula VII 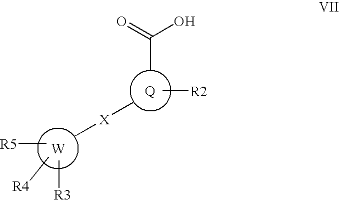
in which R2, R3, R4, R5, Q, X and W have the same meaning as defined in claim 1,
in an amide forming reaction comprising
a) conversion of said carboxylic acids into an intermediate active ester or carbonyl chloride with a suitable peptide-coupling reagent, or with thionyl chloride, oxalyl chloride, phosgene or derivatives thereof, where appropriate in the presence of a base,
b) reacting the active intermediate resulting from step a) with said amino alcohol.

10. Process according to claim 9 for preparing compounds of the formula II 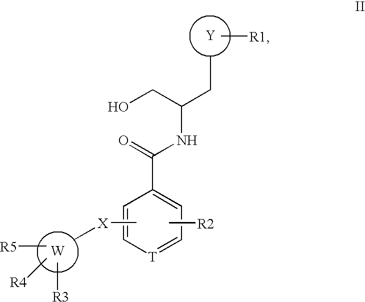
wherein an amino alcohol of the formula VI is coupled with a carboxylic acid of the formula VIII 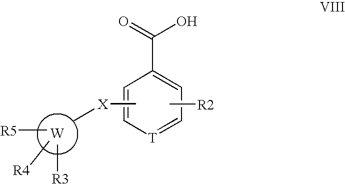
in which T is a nitrogen atom or a CH group
and R1, R2, R3, R4, R5, X, Y and W have the same meaning as defined.

11. Process according to claim 9 for preparing compounds of the formula III 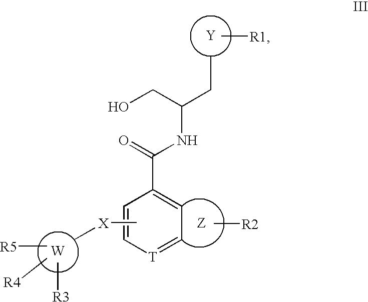
wherein an amino alcohol of the formula VI is coupled with a carboxylic acid of the formula IX 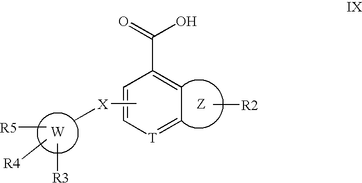
In which
T is a nitrogen atom or a CH group;
Z is a monocyclic aryl or a monocyclic heteroaryl group or a cycloalkylen, cycloalkenylen, heterocycloalkylen or heterocycloalkenylen group;
and R1, R2, R3, R4, R5, X, Y and W have the same meaning as defined.

12. Process according to claim 9 for preparing compounds of the formula IV 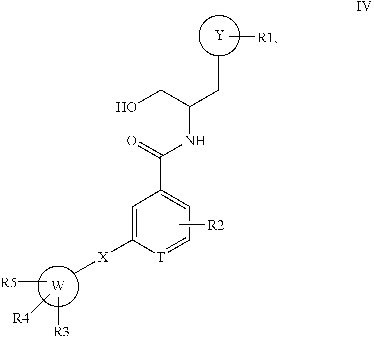
wherein an amino alcohol of the formula VI is coupled with a carboxylic acid of the formula X 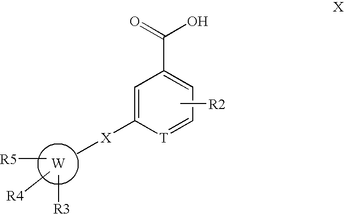
in which
T is a nitrogen atom or a CH group;
and R1, R2, R3, R4, R5, X, Y and W have the same meaning as defined.

13. Process according to claim 9 for preparing compounds of the formula V 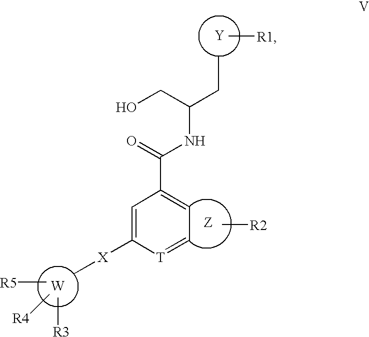
wherein an amino alcohol of the formula VI is coupled with a carboxylic acid of the formula XI 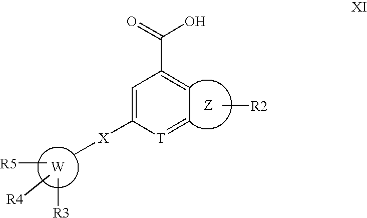
In which
T is a nitrogen atom or a CH group;
Z is a monocyclic aryl or a monocyclic heteroaryl group or a cycloalkylen, cycloalkenylen, heterocycloalkylen or heterocycloalkenylen group;
and R1, R2, R3, R4, R5, X, Y and W have the same meaning as defined.

14. Pharmaceutical compositions comprising at least one of the compounds according to claim 1 with pharmaceutically suitable excipients and/or carriers.

15. Use of the compounds of the general formula I according to claim 1 for the fertility control in men or in women.

16. Process for producing medicaments comprising at least one of the compounds of the general formula I according to claim 1 for the prevention and/or treatment of osteoporosis.
